(19) United States
(12) Patent Application Publication
(10) Pub. No.: US 2005/0227995 A1
(43) Pub. Date: Oct. 13, 2005

(54) Phenyl sulfoxides and phenyl sulfones

(76) Inventors: Martin Hendrix, Odenthal (DE); Karlheinz Baumann, Essen (DE); Rolf Grosser, Leverkusen (DE); Gerhard Konig, Arlington, MA (US); Vera Dusterhus, Bergisch (DE); Joachim Kruger, Dusseldorf (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/501,105

(22) PCT Filed: Jan. 7, 2003
(86) PCT No.: PCT/EP03/00052

§ 371 Date: Apr. 7, 2005

(30) Foreign Application Priority Data
Jan. 16, 2002 (DE) 10201392.6

(51) Int. Cl. A61K 31/495; A61K 31/4747; A61K 31/401

(52) U.S. Cl. 514/255.01; 514/278; 514/423; 548/536; 546/16; 544/384; 544/386

(57) ABSTRACT

The invention relates to phenyl sulfoxides and sulfones and processes for their preparation, and to their use for producing medicaments for the treatment and/or prophylaxis of diseases, especially Alzheimer's disease.

The invention relates to phenyl sulfoxide and sulfone derivatives and to processes for their preparation, and to their use for producing medicaments for the treatment and/or prophylaxis of diseases, especially of Alzheimer's disease.

Alzheimer's disease (AD) is a progressive neurodegenerative disorder characterized by memory loss, personality disorders, speech and orientation difficulties, impaired judgement and apathy. Up to 50% of those over 85 years of age are affected by neurodegeneration, and Alzheimer's disease is the dementia with the highest prevalence.

The most notable histopathological characteristic of Alzheimer's disease are the “senile” amyloid plaques found in the brain and especially in the regions therein associated with memory and cognition. The principal protein constituent of the plaques is the β-amyloid peptide (Aβ, βA4) with a length of 40-42 amino acids and a molecular weight of about 4 kilodaltons (kDa). Aβ is also found in the plasma and cerebrospinal fluid (CSF) of healthy individuals, but its function is unknown. In Alzheimer's patients, an increased production and/or a reduced degradation of Aβ, especially of the form with a length of 42 amino acids, leads to elevated levels of the polypeptide in plasma and CSF, followed by oligomerization of the peptide and accumulation in the brain, finally leading to the development of the plaques. Either Aβ oligomers or the plaques eventually lead to the neurodegeneration.

Aβ is produced by proteolytic processing of the amyloid precursor protein (APP) in consecutive steps by various enzymes which are called secretases. The last step in the generation of Aβ is effected by so-called γ-secretase which releases the carboxyl terminus of Aβ by cleavage of the peptide linkage. Neither the gene encoding γ-secretase nor the protein itself have yet been identified. However, the existence of this enzyme can be assumed on the basis of the available data (see also M. S. Wolfe, J. Med. Chem. 2001, 44, 2039-2060).

There is thus a need for substances which prevent the production of Aβ by proteolytic processing of APP.

CAPLUS 1986, 185969 (JP-A-60252430) and CAPLUS 1988, 21523 (JP-A-62175456) describe substituted phenyl benzyl sulfones as intermediates for the preparation of, for example, insecticides.

Phenyl sulfone derivatives as γ-secretase inhibitors are described in WO 02/081433 and WO 02/081435. Structurally different γ-secretase inhibitors are disclosed, for example, in Rishton et al., J. Med. Chem. 2000, 43, 2297-2299 and in WO 01/77086, WO 01/77144, WO 01/53255 and WO 00/50391.

The present invention relates to compounds of the formula 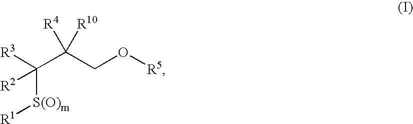
in which
R¹ and R² are independently of one another phenyl which is optionally substituted by radicals selected from the group of halogen, cyano, trifluoromethyl, trifluoromethoxy, C₁-C₆-alkyl, C₃-C₈-cycloalkyl, C₁-C₆-alkoxy and C₁-C₆-alkylthio,
R³ and R⁴ are independently of one another hydrogen, C₁-C₆-alkyl or C₃-C₈-cycloalkyl, which are optionally substituted by hydroxy,
m is 1 or 2,
R⁵ is hydrogen,
   or a radical of the formula CO—NR⁶R⁷ in which
   R⁶ and R⁷ are independently of one another hydrogen, C₁-C₆-alkyl, C₃-C₈-cycloalkyl, benzyl, phenethyl, phenyl or 5- to 6-membered heteroaryl, where C₁-C₆-alkyl, C₃-C₈-cycloalkyl, phenyl or 5- to 6-membered heteroaryl are optionally substituted by radicals independently of one another selected from the group of hydroxy, halogen, C₁-C₆-alkylamino, aminosulfonyl, aminocarbonyl, cyano, formamido, acetamido, C₁-C₆-alkyl, C₁-C₆-alkoxy, C₃-C₈-cycloalkyl, hydroxycarbonyl, C₁-C₆-alkoxycarbonyl and 5- to 6-membered heteroaryl, and
      benzyl and phenethyl are optionally substituted by radicals independently of one another selected from the group of hydroxy, halogen, aminocarbonyl, C₁-C₆-alkylamino, aminosulfonyl, cyano, formamido, acetamido, C₁-C₆-alkyl, C₁-C₆-alkoxy, C₃-C₈-cycloalkyl and 5- to 6-membered heteroaryl,
      or in which
      the group NR⁶R⁷
      is a 4- to 10-membered heterocyclyl radical which is linked via the nitrogen atom and which is optionally substituted by radicals independently of one another selected from the group of C₁-C₆-alkyl, C₁-C₆-alkoxy, 1,3-dioxapropane-1,3-diyl, 1,4-dioxabutane-1,4-diyl, oxo, C₃-C₈-cycloalkyl, hydroxy, halogen, cyano, C₁-C₆-alkylcarbonyl, C₃-C₈-cycloalkylcarbonyl, phenylcarbonyl, formamido, aminosulfonyl, C₁-C₆-alkoxycarbonyl, aminocarbonyl, phenyl and 5- to 6-membered heteroaryl,
      where phenyl is optionally substituted by radicals independently of one another selected from the group of halogen, cyano, trifluoromethyl, trifluoromethoxy, C₁-C₆-alkyl, C₁-C₆-alkoxy and C₁-C₆-alkylsulfonamino, and
         C₁-C₆-alkyl is optionally substituted by radicals independently of one another selected from the group of hydroxy, C₁-C₆-alkoxy, phenyl and 5- to 6-membered heteroaryl, and
         C₁-C₆-alkylcarbonyl is optionally substituted by radicals independently of one another selected from the group of hydroxy and C₁-C₆-alkoxy,
      and where 4- to 10-membered heterocyclyl is optionally benzo-substituted, or
   a radical of the formula CO—OR⁸ in which
   R⁸ is C₁-C₆-alkyl or C₃-C₈-cycloalkyl, which are optionally substituted by radicals independently of one another selected from the group of hydroxy, halogen, aminosulfonyl, aminocarbonyl, cyano, formamido, acetamido, C₁-C₆-alkyl, C₁-C₆-alkoxy, C₃-C₈-cycloalkyl, C₁-C₆-alkylcarbonyl, phenyl and 5- to 6-membered heteroaryl, or
   a radical of the formula CO—R⁹ in which
   R⁹ is C₁-C₆-alkyl, C₃-C₈-cycloalkyl, C₆-C₁₀-aryl or 5- to 10-membered heteroaryl, which are optionally substituted by radicals selected from the group of hydroxy, hydroxycarbonyl, halogen, aminosulfonyl, carboxamido, cyano, formamido, acetamido, C₁-C₆-alkyl, C₁-C₆-alkoxy, C₃-C₈-cycloalkyl, C₁-C₆-alkylcarbonyl, phenyl and 5- to 6-membered heteroaryl,
R¹⁰ is hydrogen or C₁-C₆-alkyl,
   and the salts, solvates and solvates of the salts thereof.

The compounds of the invention may also be in the form of their salts, solvates or solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid and benzoic acid.

Physiologically acceptable salts of the compounds (I) also include salts of conventional bases, such as by way of example and preferably alkali metal salts (e.g. sodium and potassium salts), alkaline earth metal salts (e.g. calcium and magnesium salts) and ammonium salts derived from ammonia or organic amines having 1 to 16 C atoms, such as by way of example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabiethylamine, arginine, lysine, ethylenediamine and methylpiperidine.

Solvates refers for the purposes of the invention to those forms of the compounds which form a complex in the solid or liquid state through coordination with solvent molecules. Hydrates are a specific form of solvates in which the coordination takes place with water.

For the purposes of the present invention, the radicals have the following meaning unless specified otherwise:

C₁-C₆-Alkylamino stands for a straight-chain or branched mono- or dialkylamino radical having 1 to 6, preferably 1 to 4 and particularly preferably having 1 to 3, carbon atoms. Nonlimiting examples include methylamino, ethylamino, n-propylamino, isopropylamino, tert-butylamino, n-pentylamino, n-hexylamino, dimethylamino, diethylamino, di-n-propylamino, diisopropylamino, di-t-butylamino, di-n-pentylamino, di-n-hexylamino, ethylmethylamino, isopropylmethylamino, n-butylethylamino, n-hexyl-1-pentylamino.

C₁-C₆-Alkylcarbonyl stands for a straight-chain or branched alkylcarbonyl radical having 1 to 6, preferably 1 to 4, carbon atoms. Nonlimiting examples include formyl, acetyl, propanoyl, butanoyl, isobutanoyl, pentanoyl, isopentanoyl and hexanoyl. Acetyl and propanoyl are particularly preferred.

C₁-C₆- and C₁-C₄-alkyl stand for a straight-chain or branched alkyl radical respectively having 1 to 6 and 1 to 4, preferably 1 to 4 and particularly preferably having 1 to 3, carbon atoms. Nonlimiting examples include methyl, ethyl, n-propyl, isopropyl, tert-butyl, n-pentyl and n-hexyl.

C₁-C₆-Alkylsulfonamino stand for a straight-chain or branched alkylsulfonylamino radical having 1 to 6, with preference for a straight-chain or branched alkanesulfonylamino radical having 1 to 4, particularly preferably having 1 to 3, carbon atoms. Nonlimiting examples include methanesulfonylamino, ethanesulfonylamino, n-propanesulfonylamino, isopropanesulfonylamino, tert-butanesulfonylamino, n-pentanesulfonamino, n-hexanesulfonamino.

C₁-C₆-Alkoxycarbonyl stands for a straight-chain or branched alkoxycarbonyl radical having 1 to 6, preferably 1 to 4, particularly preferably having 1 to 3, carbon atoms. Nonlimiting examples include methoxycarbonyl, ethoxycarbonyl, n-propoxycarbonyl, isopropoxycarbonyl and tert-butoxycarbonyl.

C₁-C₆-Alkoxy stands for a straight-chain or branched alkoxy radical having 1 to 6, preferably 1 to 4 and particularly preferably having 1 to 3, carbon atoms. Nonlimiting examples include methoxy, ethoxy, n-propoxy, isopropoxy, tert-butoxy, n-pentoxy and n-hexoxy.

C₁-C₆-Alkylthio stands for a straight-chain or branched alkylthio radical having 1 to 6, preferably 1 to 4 and particularly preferably having 1 to 3, carbon atoms. Nonlimiting examples include methylthio, ethylthio, n-propylthio, isopropylthio, tert-butylthio, n-pentylthio and n-hexylthio.

C₆-C₁₀-Aryl stands for an aromatic radical having 6 to 10 carbon atoms. Preferred aryl radicals are phenyl and naphthyl.

C₃-C₈-Cycloalkylcarbonyl stands for cyclopropylcarbonyl, cyclopentylcarbonyl, cyclobutylcarbonyl, cyclohexylcarbonyl, cycloheptylcarbonyl or cyclooctylcarbonyl. The following may be mentioned as preferred: cyclopropylcarbonyl, cyclopentylcarbonyl and cyclohexylcarbonyl.

C₃-C₈-Cycloalkyl stands for cyclopropyl, cyclopentyl, cyclobutyl, cyclohexyl, cycloheptyl or cyclooctyl. The following may be mentioned as preferred: cyclopropyl, cyclopentyl and cyclohexyl.

5- to 6-membered heteroaryl stands for an aromatic radical having 5 to 6 ring atoms and up to 4 heteroatoms from the series S, O and/or N. The heteroaryl radical may be linked via a carbon atom or heteroatom. Nonlimiting examples include thienyl, furyl, pyrrolyl, thiazolyl, oxazolyl, imidazolyl, tetrazolyl, pyridyl, pyrimidinyl, and pyridazinyl.

5- to 10-membered heteroaryl stands for an aromatic, mono- or bicyclic radical having 5 to 10 ring atoms and up to 5 heteroatoms from the series S, O and/or N. 5- to 6-membered heteroaryls having up to 4 heteroatoms are preferred. The heteroaryl radical may be linked via a carbon atom or heteroatom. Nonlimiting examples include thienyl, furyl, pyrrolyl, thiazolyl, oxazolyl, imidazolyl, tetrazolyl, pyridyl, pyrimidinyl, pyridazinyl, indolyl, indazolyl, benzofuranyl, benzothiophenyl, quinolinyl, isoquinolinyl.

The 4- to 10-membered heterocyclyl radical which is linked via a nitrogen atom stands for a mono- or polycyclic, preferably mono- or bicyclic, nonaromatic heterocyclic radical having 4 to 10, preferably 5 to 8, ring atoms, with at least one nitrogen atom via which the heterocyclyl radical is linked, and having up to 2, preferably up to 1, further heteroatoms and/or hetero groups from the series N, O, S, SO, and SO₂. The heterocyclyl radical may be saturated or partially unsaturated. Preference is given to 5- to 8-membered, monocyclic saturated heterocyclyl radicals having up to two heteroatoms from the series O, N and S, such as by way of example and preferably tetrahydrofuran-2-yl, pyrrolidin-2-yl, pyrrolidin-3-yl, pyrrolinyl, piperidinyl, morpholinyl, perhydroazepinyl.

If radicals in the compounds of the invention are substituted, the radicals may, unless specified otherwise, have one or more identical or different substituents. Substitution by up to three identical or different substituents is preferred. Substitution by one substituent is very particularly preferred.

Preference is given to compounds of the formula (I) in which
R¹ and R² are independently of one another phenyl which is optionally substituted by radicals selected from the group of halogen, cyano, trifluoromethyl,
R³ and R⁴ are independently of one another hydrogen, C₁-C₄-alkyl or C₃-C₆-cycloalkyl, which are optionally substituted by hydroxy,
m is 1 or 2,
R⁵ is hydrogen, or
   a radical of the formula CO—NR⁶R⁷ in which
   R⁶ is hydrogen, C₁-C₄-alkyl,
   R⁷ is hydrogen, C₁-C₄-alkyl, C₃-C₆-cycloalkyl, benzyl, phenethyl or phenyl, where C₁-C₄-alkyl, C₃-C₆-cycloalkyl and phenyl are optionally substituted by radicals independently of one another selected from the group of hydroxy, halogen, aminocarbonyl, hydroxycarbonyl, cyano, C₁-C₄-alkylamino, C₁-C₄-alkyl, C₁-C₄-alkoxy, C₃-C₆-cycloalkyl, C₁-C₄-alkoxycarbonyl and 5- to 6-membered heteroaryl, and
      benzyl and phenethyl are optionally substituted by radicals independently of one another selected from the group of hydroxy, halogen, aminocarbonyl, cyano, C₁-C₄-alkylamino, C₁-C₄-alkyl, C₁-C₄-alkoxy, C₃-C₆-cycloalkyl and 5- to 6-membered heteroaryl,
   or in which
   the group NR⁶R⁷
      is a 5- to 6-membered heterocyclyl radical which is linked via the nitrogen atom and which is optionally substituted by radicals independently of one another selected from the group of C₁-C₄-alkyl, C₁-C₄-alkoxy, 1,3-dioxapropane-1,3-diyl, 1,4-dioxabutane-1,4-diyl, oxo, C₃-C₆-cycloalkyl, hydroxy, halogen, C₁-C₄-alkylcarbonyl, C₃-C₆-cycloalkylcarbonyl, phenylcarbonyl, C₁-C₄-alkoxycarbonyl, phenyl and 5- to 6-membered heteroaryl,
      where phenyl is optionally substituted by radicals independently of one another selected from the group of halogen, cyano, trifluoromethyl, trifluoromethoxy, C₁-C₄-alkyl, C₁-C₄-alkoxy and C₁-C₄-alkylsulfonamino, and
      C₁-C₄-alkyl is optionally substituted by radicals independently of one another selected from the group of hydroxy and phenyl, and
      C₁-C₄-alkylcarbonyl is optionally substituted by radicals independently of one another selected from the group of hydroxy and C₁-C₄-alkoxy, or
   a radical of the formula CO—R⁹ in which
   R⁹ is C₁-C₄-alkyl, C₃-C₈-cycloalkyl, phenyl or 5- to 6-membered heteroaryl, which are optionally substituted by radicals selected from the group of hydroxy, hydroxycarbonyl, halogen, cyano, acetamido, C₁-C₄-alkyl, C₁-C₄-alkoxy, C₃-C₆-cycloalkyl, C₁-C₄-alkylcarbonyl, phenyl and 5- to 6-membered heteroaryl,
R¹⁰ is hydrogen or C₁-C₄-alkyl,
   and the salts, solvates and solvates of the salts thereof.

Particular preference is given to compounds of the formula (I) in which
R¹ is phenyl which is optionally substituted by radicals selected from the group of fluorine, chlorine, bromine, cyano, trifluoromethyl,
R² is phenyl which is optionally substituted by fluorine,
R³ is hydrogen or C₁-C₄-alkyl,
R⁴ is hydrogen or C₁-C₄-alkyl which is optionally substituted by hydroxy
R⁵ is hydrogen, or
   a radical of the formula CO—NR⁶R⁷ in which
   R⁶ is hydrogen, C₁-C₄-alkyl,
   R⁷ is C₁-C₄-alkyl, C₃-C₆-cycloalkyl, benzyl, phenethyl or phenyl, where C₁-C₄-alkyl, C₃-C₆-cycloalkyl, and phenyl are optionally substituted by radicals independently of one another selected from the group of hydroxy, fluorine, chlorine, aminocarbonyl, hydroxycarbonyl, cyano, dimethylamino, methoxy, ethoxy, C₁-C₄-alkoxycarbonyl or thienyl, and
      benzyl and phenethyl are optionally substituted by radicals independently of one another selected from the group of hydroxy, fluorine, chlorine, aminocarbonyl, cyano, dimethylamino, methoxy, ethoxy or thienyl,
   or in which
   the group NR⁶R⁷
      is a 5- to 6-membered heterocyclyl radical which is linked via the nitrogen atom and which is optionally substituted by radicals independently of one another selected from the group of C₁-C₄-alkyl, 1,3-dioxapropane-1,3-diyl, 1,4-dioxabutane-1,4-diyl, oxo, hydroxy, C₁-C₄-alkylcarbonyl, C₃-C₆-cycloalkylcarbonyl, phenylcarbonyl, C₁-C₄-alkoxycarbonyl, phenyl and 6-membered heteroaryl,
      where phenyl is optionally substituted by radicals independently of one another selected from the group of fluorine, chlorine, cyano, trifluoromethyl, trifluoromethoxy, C₁-C₄-alkyl, C₁-C₄-alkoxy and
      C₁-C₄-alkylsulfonamino, and
      C₁-C₄-alkyl is optionally substituted by radicals independently of one another selected from the group of hydroxy and phenyl, and C₁-C₄-alkylcarbonyl is optionally substituted by radicals independently of one another selected from the group of hydroxy and methoxy, or
   a radical of the formula CO—R⁹ in which
   R⁹ is phenyl,
R¹⁰ is hydrogen or C₁-C₃-alkyl,
   and the salts, solvates and solvates of the salts thereof.

Very particular preference is given to compounds of the following formulae 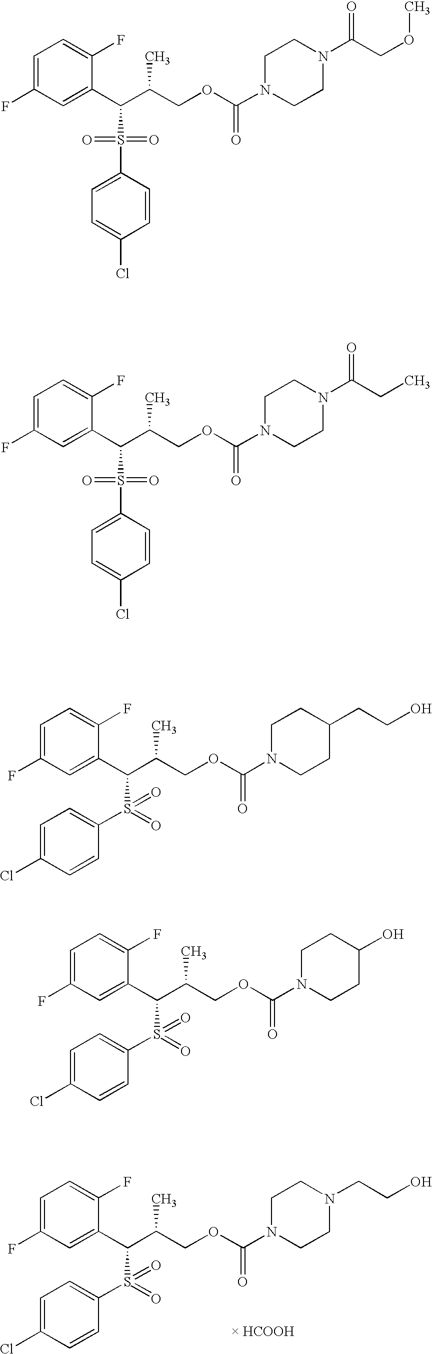 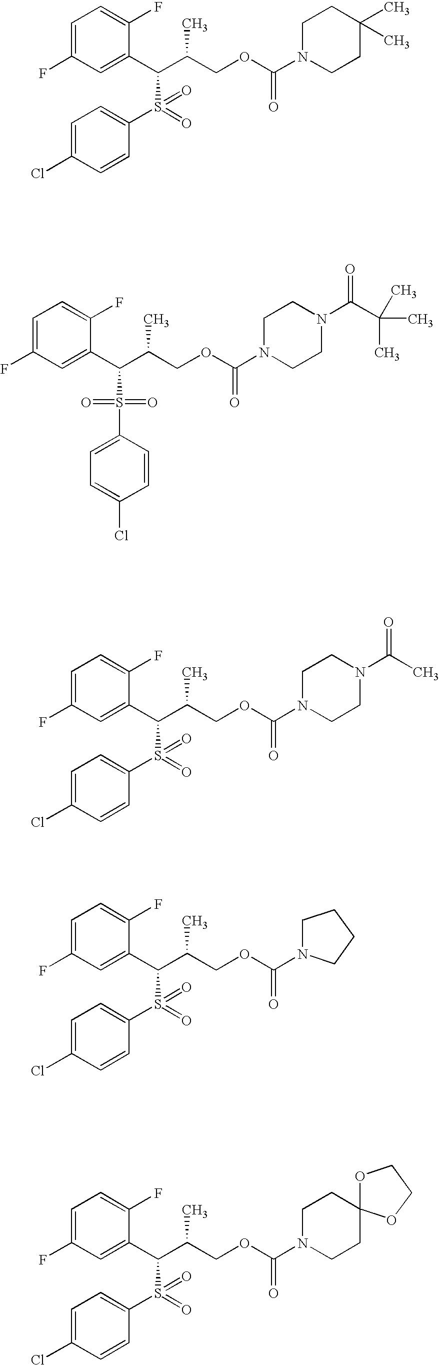 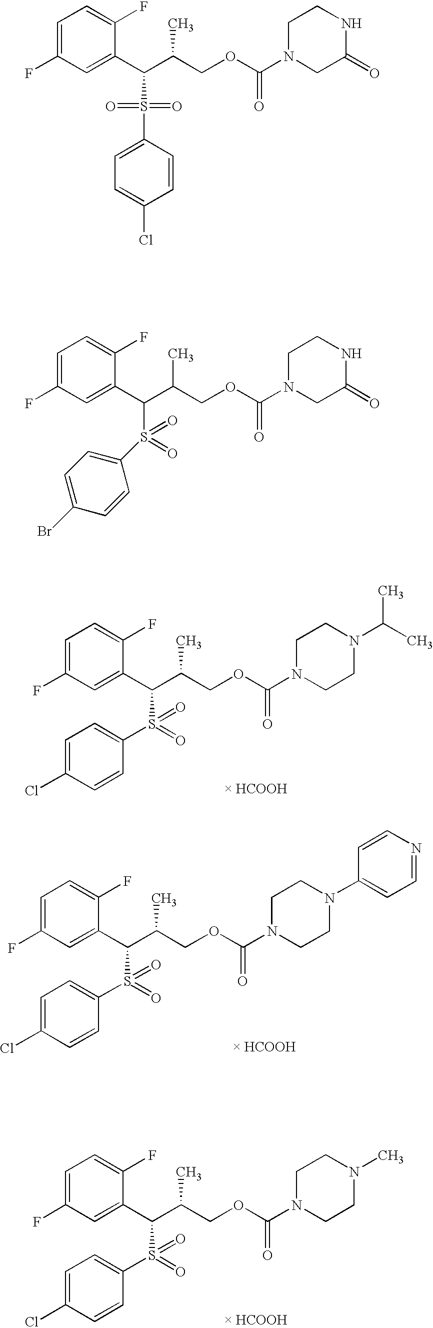 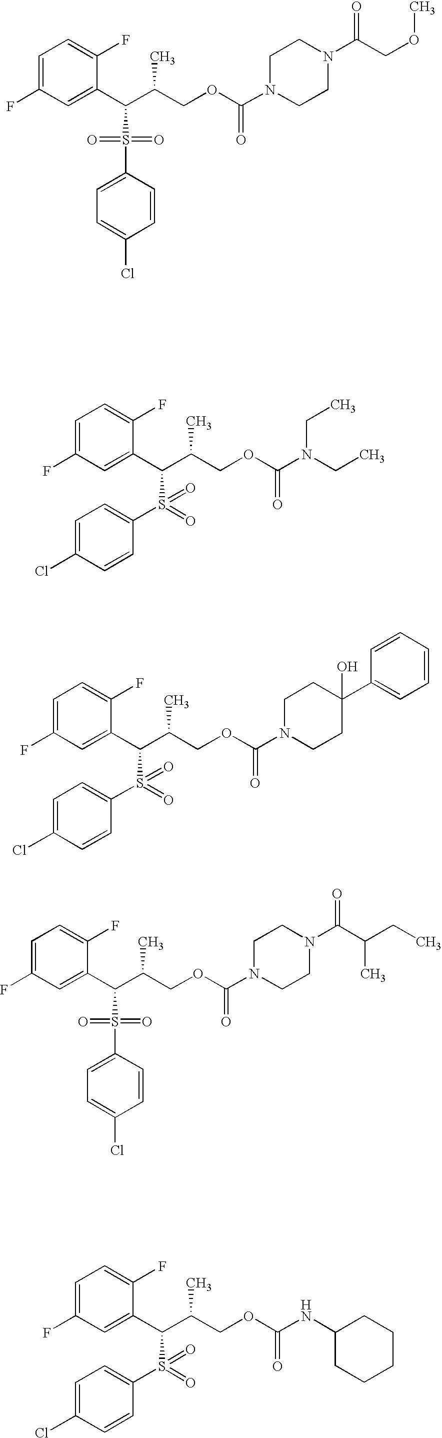 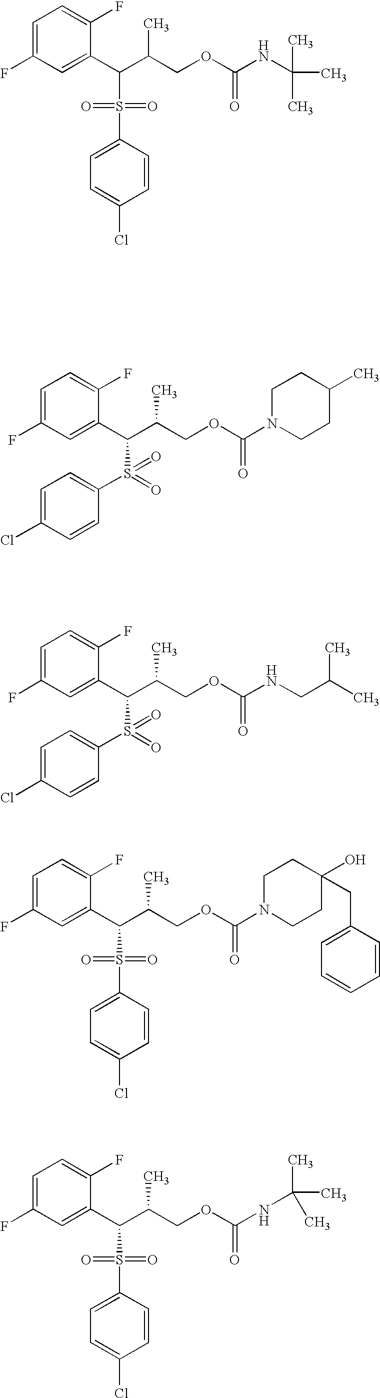 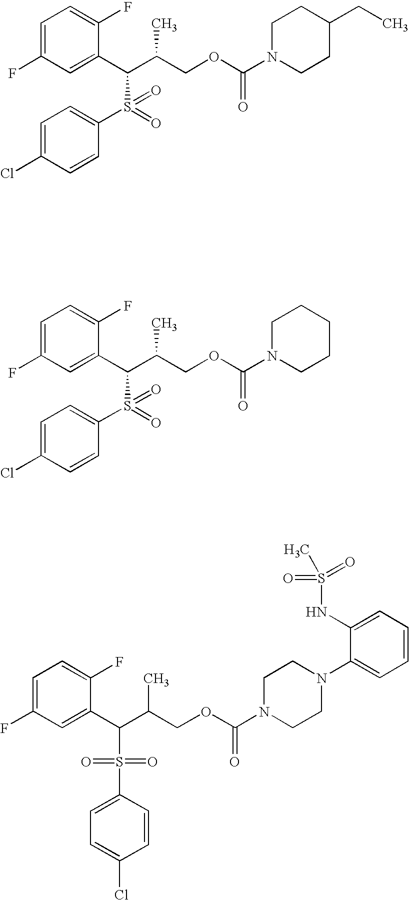
and the salts, solvates and solvates of the salts thereof.

The present invention also relates to compounds of the formula 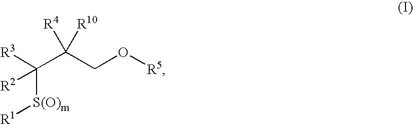
in which
R¹ and R² are independently of one another phenyl, which is optionally substituted by radicals selected from the group of halogen, cyano, trifluoromethyl, trifluoromethoxy, C₁-C-alkyl, C₃-C₈-cycloalkyl, C₁-C₆-alkoxy and C₁-C₆-alkylthio,
R³ and R⁴ are independently of one another hydrogen, C₁-C₆-alkyl or C₃-C₈-cycloalkyl,
m is 1 or 2, and
R⁵ is hydrogen,
   is a radical of the formula CO—NR⁶R⁷
   in which R⁶ and R⁷ are independently of one another hydrogen, C₁-C₆-alkyl, C₃-C₈-cycloalkyl, phenyl or 5- to 6-membered heteroaryl, or
   in which the group NR⁶R⁷ is a 4- to 10-membered heterocyclyl radical which is linked via a nitrogen atom,
   where alkyl, cycloalkyl, phenyl, heteroaryl and heterocyclyl are optionally substituted by radicals selected from the group of hydroxy, halogen, aminosulfonyl, carboxamido, cyano, formamido, acetamido, C₁-C₆-alkyl, C₁-C₆-alkoxy, C₃-C₈-cycloalkyl, C₁-C₆-alkanoyl, phenyl and 5- to 6-membered heteroaryl,
   and where heterocyclyl is optionally is benzo-substituted,
   is a radical of the formula CO—OR⁸
   in which R⁸ is C₁-C₆-alkyl or C₃-C₈-cycloalkyl,
   where alkyl and cycloalkyl are optionally substituted by radicals selected from the group of hydroxy, halogen, aminosulfonyl, carboxamido, cyano, formamido, acetamido, C₁-C₆-alkyl, C₁-C₆-alkoxy, C₃-C₈-cycloalkyl, C₁-C₆-alkanoyl, phenyl and 5- to 6-membered heteroaryl, or
   is a radical of the formula CO—R⁹,
   in which R⁹ is C₁-C₆-alkyl, C₃-C₈-cycloalkyl, C₆-C₁₀-aryl or 5- to 10-membered heteroaryl,
   where alkyl, cycloalkyl, aryl and heteroaryl are optionally substituted by radicals selected from the group of hydroxy, halogen, aminosulfonyl, carboxamido, cyano, formamido, acetamido, C₁-C₆-alkyl, C₁-C₆-alkoxy, C₃-C₈-cycloalkyl, C₁-C₆-alkanoyl, phenyl and 5- to 6-membered heteroaryl,
      and the salts, solvates and solvates of the salts thereof.

Preference is given to compounds of the formula (I)

in which

R¹ and R² are independently of one another phenyl which is optionally substituted once to three times by radicals selected from the group of halogen, cyano, trifluoromethyl, trifluoromethoxy and C₁-C₆-alkyl,
and R³, R⁴, m and R⁵ have the meaning indicated above or below.

Particular preference is given to compounds of the formula (I)

in which

R¹ is 2-fluorophenyl which is optionally additionally substituted once to twice by radicals selected from the group of fluorine, chlorine, cyano, trifluoromethyl, methyl and ethyl,
and R², R³, R⁴, m and R⁵ have the meaning indicated above or below.

Very particular preference is given to compounds of the formula (I)

in which

R¹ is 2,4-difluorophenyl,
and R², R³, R⁴, m and R⁵ have the meaning indicated above or below.

Particular preference is likewise given to compounds of the formula (I)

in which

R² is 4-chlorophenyl which is optionally additionally substituted once to twice by radicals selected from the group of fluorine, chlorine, cyano, trifluoromethyl, methyl and ethyl,
and R¹, R³, R⁴, m and R⁵ have the meaning indicated above or below.

Very particular preference is given to compounds of the formula (I)

in which

R² is 4-chlorophenyl,
and R¹, R³, R⁴, m and R⁵ have the meaning indicated above or below.

Preference is likewise given to compounds of the formula (I)

in which

R³ is hydrogen or methyl,
and R¹, R², R⁴, m and R⁵ have the meaning indicated above or below.

Particular preference is given to compounds of the formula (I)

in which

R³ is hydrogen,
and R¹, R², R⁴, m and R⁵ have the meaning indicated above or below.

Preference is likewise given to compounds of the formula (I)

in which

R⁴ is hydrogen or C₁-C₄-alkyl,
and R¹, R², R³, m and R⁵ have the meaning indicated above or below.

Particular preference is given to compounds of the formula (I)

in which

R⁴ is methyl or ethyl,
and R¹, R², R⁴, m and R⁵ have the meaning indicated above or below.

Preference is likewise given to compounds of the formula (I)

in which

m is 1,
and R¹, R², R³, R⁴ and R⁵ have the meaning indicated above or below.

Preference is likewise given to compounds of the formula (I)

in which

R⁵ is hydrogen or a radical of the formula CO—NR⁶R⁷,
   in which R⁶ and R⁷ are independently of one another hydrogen, C₁-C₆-alkyl, C₃-C₈-cycloalkyl or benzyl, or
   in which the group NR⁶R⁷ is a 5- to 8-membered heterocyclyl radical which is linked via a nitrogen atom,
and R¹, R², R⁴ and m have the meaning indicated above or below.

Particular preference is given to compounds of the formula (I)

in which

R⁵ is a radical of the formula CO—NR⁶R⁷,
   in which R⁶ and R⁷ are independently of one another hydrogen, C₁-C₆-alkyl, C₃-C₈-cycloalkyl or benzyl, or
   in which the group NR⁶R⁷ is pyrrolidin-1-yl, piperidin-1-yl, morpholin-1-yl, thiomorpholin-1-yl, piperazin-1-yl, 4-methyl-piperazin-1-yl or 4-ethylpiperazin-1-yl,
and R¹, R², R⁴ and m have the meaning indicated above or below.

Very particular preference is given to combinations of two or more of the abovementioned preference ranges.

Very particular preference is likewise given to compounds of the formula (I)

in which

R¹ is 2-fluorophenyl which is optionally additionally substituted once to twice by radicals selected from the group of fluorine, chlorine, cyano, trifluoromethyl, methyl and ethyl,
R² is 4-chlorophenyl which is optionally additionally substituted once to twice by radicals selected from the group of fluorine, chlorine, cyano, trifluoromethyl, methyl and ethyl,
R³ is hydrogen,
R⁴ is hydrogen or C₁-C₄-alkyl,
m is 1 or 2, and
R⁵ is a radical of the formula CO—NR⁶R⁷,
   in which R⁶ and R⁷ are independently of one another hydrogen, C₁-C₆-alkyl, C₃-C₈-cycloalkyl or benzyl, or
   in which the group NR⁶R⁷ is pyrrolidin-1-yl, piperidin-1-yl, morpholin-1-yl, thiomorpholin-1-yl, piperazin-1-yl, 4-methylpiperazin-1-yl or 4-ethylpiperazin-1-yl.

Preference is likewise given to compounds of the formula (I)

in which

R¹⁰ is hydrogen or C₁-C₃-alkyl,
and R¹-R⁴ and m have the meanings indicated above.

The invention further relates to processes for preparing the compounds of the invention, characterized in that
[A] compounds of the formula 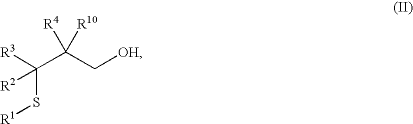
   in which R¹ to R⁴ and R¹⁰ have the meanings indicated above,
   are first converted with appropriate equivalents of a suitable oxidizing agent such as, for example, peroxides or peracids, preferably meta-chloroperbenzoic acid (mCPBA) into compounds of the formula 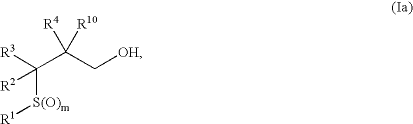
   in which R¹ to R⁴, R¹⁰ and m have the meanings indicated above,
   and the latter are then reacted in an acylation step, where appropriate in the presence of a base, with a compound of the formula
      R^{5a}—X (III),
   in which
   R^{5a} has the meanings indicated above for R⁵ with the exception of hydrogen, and
   X is a suitable leaving group such as, for example, halogen, or
[B] compounds of the formula (II) are first converted with a compound of the formula (III), where appropriate in the presence of a base, into compounds of the formula 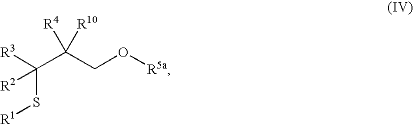
   in which
   R¹ to R⁴, R^{5a} and R¹⁰ have the meanings indicated above,
   and the latter are then reacted with appropriate equivalents of a suitable oxidizing agent, preferably meta-chloroperbenzoic acid, or
[C] compounds of the formula 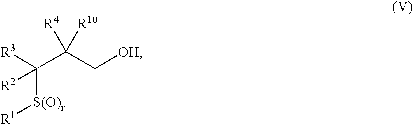
   in which
   R¹ to R⁴ and R¹⁰ have the meanings indicated above, and
   r is zero, 1 or 2,
   are first reacted, where appropriate in the presence of a base, with a compound of the formula 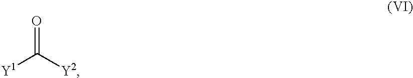
   in which
   Y¹ and Y² are identical or different and are a suitable leaving group such as, for example, halogen, —OCCl₃ or a group of the formula 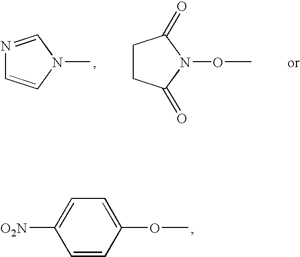
   to give compounds of the formula 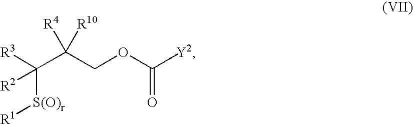
   in which
   R¹ to R⁴, R¹⁰, r and Y² have the meanings indicated above,
   and the latter are then converted, where appropriate in the presence of a base and/or of a suitable catalyst, with a compound of the formulae 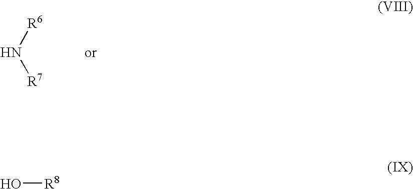
   in which
   R⁶, R⁷ and R⁸ have the meanings indicated above,
   into compounds of the formulae 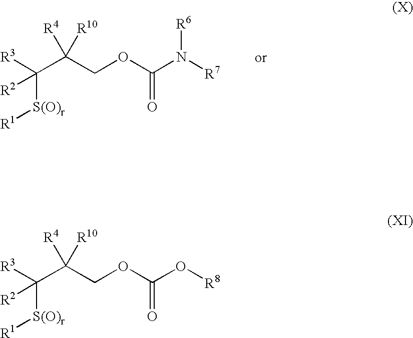
   in which
   R¹ to R⁴, R⁶ to R⁸, R¹⁰ and r have the meanings indicated above,
   and the latter are then, if r is zero, reacted with appropriate equivalents of a suitable oxidizing agent, preferably meta-chloroperbenzoic acid,
      and the resulting compounds (I) and (Ia) where appropriate are converted with the appropriate solvents and/or bases or acids into their solvates, salts and/or solvates of the salts.

The compounds (II) can be prepared by firstly reacting compounds of the formula 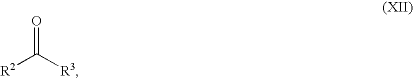
in which R² and R³ have the meanings indicated above,
with a compound of the formula 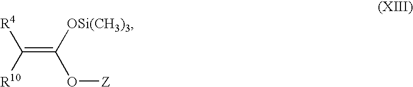
in which R⁴ and R¹⁰ have the meanings indicated above, and
Z is C₁-C₄-alkyl,
   in the presence of a Lewis acid, preferably titanium tetrachloride, in an inert solvent to give compounds of the formula 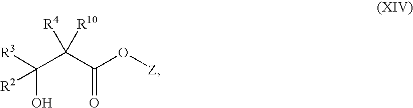
   in which R² to R⁴, R¹⁰ and Z have the meanings indicated above,
   the latter are then converted in inert solvents in the presence of triphenylphosphine and of a di(C₁-C₄-alkyl) azodicarboxylate under Mitsunobu conditions with a thiol of the formula
   R¹—SH (XV),
   in which R¹ has the meaning indicated above,
   into compounds of the formula 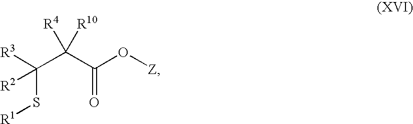
   in which R¹ to R⁴, R¹⁰ and Z have the meanings indicated above,
   and the latter are subsequently reacted with a suitable reducing agent such as, for example, complex metal hydrides, preferably lithium aluminum hydride, in an inert solvent.

Compounds of the formula (II) in which R¹⁰ is hydrogen can additionally be prepared by converting compounds of the formula 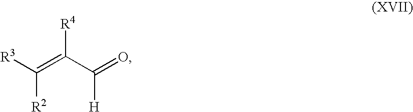
in which R² to R⁴ have the meanings indicated above,
with a thiol of the formula (XV) into compounds of the formula 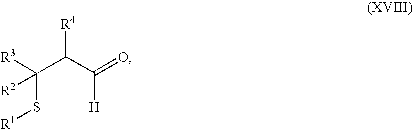
in which R¹ to R⁴ have the meanings indicated above,
and then reacting the latter with a suitable reducing agent such as, for example, complex metal hydrides, preferably sodium borohydride. The process steps (XVII)→(XVIII)→(II) can moreover be carried out with isolation of the intermediate (XVIII) or in a “one-pot” process [cf., for example, Y.-H. Chang, H. W. Pinnick, J. Org. Chem. 43, 373-374 (1978)].

Compounds of the formula (II) in which R⁴ and R¹⁰ are hydrogen can additionally be prepared by first deprotonating compounds of the formula 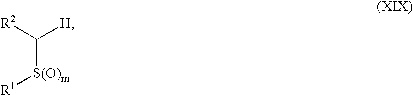
in which R¹, R² and m have the meanings indicated above,
with a suitable base, preferably n-butyllithium, in an inert solvent subsequently reacted with a compound of the formula 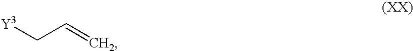
in which
Y³ is a suitable leaving group such as, for example, halogen, mesylate, tosylate or triflate,
   to give compounds of the formula 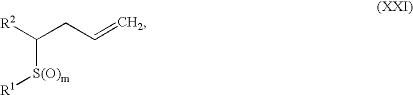
   in which R¹, R² and m have the meanings indicated above,
   deprotonating the compounds (XXI) where appropriate in an additional step once again with a suitable base, preferably sodium hydride, in an inert solvent, and reacting with a compound of the formula
   R³—Y⁴ (XXII),
   in which
R³ has the meaning indicated above but is not hydrogen, and
Y⁴ is a suitable leaving group such as, for example, halogen, mesylate, tosylate or triflate,
   to give compounds of the formula 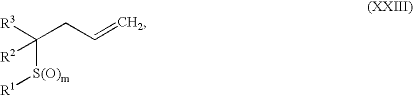
   in which R¹, R², R³ and m have the meanings indicated above,
   and then converting the compounds (XXI) and (XXI) by means of a suitable oxidizing agent such as potassium permanganate or osmium tetroxide, preferably osmium tetroxide, followed in a second step by a reduction with a complex hydride, preferably sodium borohydride, in an inert solvent into compounds of the formula 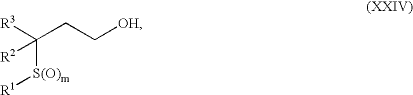
   in which R¹, R², R³ and m have the meanings indicated above.

In analogy to the process (XXI)+(XXII)→(XXIII) described above, the compounds (Ia) can also be prepared by firstly converting compounds of the formula 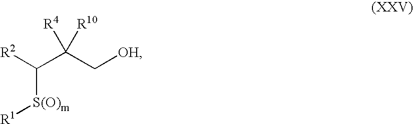
in which R¹, R², R⁴, R¹⁰ and m have the meanings indicated above,
by customary literature methods into compounds of the formula 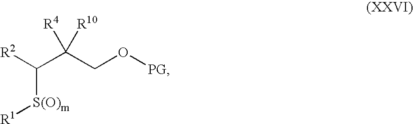
in which R¹, R², R⁴, R¹⁰ and m have the meanings indicated above, and
PG is a suitable hydroxy protective group such as, for example, trimethylsilyl or tert-butyldimethylsilyl,
   subsequently deprotonating with a suitable base, preferably sodium hydride, in an inert solvent, and reacting with a compound of the formula (XXII) to give compounds of the formula 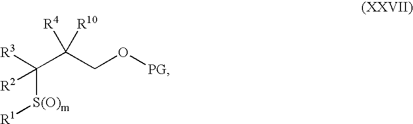
   in which R¹ to R⁴, R¹⁰, m and PG have the meanings indicated above, and finally eliminating the hydroxy protective group by customary literature methods.

The compounds (III), (VI), (VIII), (IX), (XII), (XIII), (XV), (XVII), (XIX), (XX) and (XXII) are commercially available, known from the literature or can be prepared by customary literature methods. The compounds (V) correspond to those of the formula (II) or (Ia), and the compounds (XXV) to those of the formula (Ia); they can in each case be prepared as described therefor.

Various methods for acylating a hydroxy group for introducing the radicals R^{5a} [process steps (Ia)→(I) and (II)→(IV)] are known to the skilled worker or described in the relevant literature (e.g. Houben-Weyl). For example, reaction with an acid chloride in an inert solvent in the presence of a base such as, for example, pyridine has proved useful. Suitable for introducing carbasoyl radicals is, for example, reaction with para-nitrophenyl chloroformate and subsequent reaction of the resulting intermediate with an amine. Other acylating agents such as, for example, carbonyldiimidazole are likewise suitable for this purpose. The compounds of the invention can be synthesized by linking the acylation in either sequence with the oxidation of the sulfide group, i.e. first acylation and then oxidation, or first oxidation and then acylation.

Suitable solvents for the oxidation in process steps [A] (II)→(Ia), [B] (IV)→(I) and [C] (X)/(XI)→(I) are inert organic solvents which are not changed under the reaction conditions. These include halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, esters such as ethyl acetate, ketones such as acetone, amides such as dimethylformamide or nitriles such as acetonitrile. It is likewise possible to employ mixtures of said solvents. Dichloromethane is particularly preferred.

The oxidation generally takes place in a temperature range from −30° C. to +50° C., preferably in a temperature range from 0° C. to +25° C.

Suitable solvents for the acylation in process steps [A] (Ia)+(III)→(I) and [B] (II)+(III)→(IV) are likewise inert organic solvents. These include halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, nitroalkanes such as nitromethane, esters such as ethyl acetate, ketones such as acetone, heteroaromatic compounds such as pyridine, amides such as dimethylformamide, dialkyl sulfoxides such as dimethyl sulfoxide, or nitriles such as acetonitrile. It is likewise possible to employ mixtures of said solvents. Tetrahydrofuran, acetonitrile, dimethylformamide or mixtures thereof are preferred.

Customary inorganic or organic bases are suitable as base for the acylation step. These preferably include alkali metal or alkaline earth metal carbonates such as sodium, potassium or calcium carbonate, alkali metal hydrides such as sodium hydride, amides such as lithium bis(trimethylsilyl)amide or lithium diisopropylamide, organic amines such as pyridine, 4-N,N-dimethylaminopyridine, 4-pyrrolidinopyridine, triethylamine, ethyldiisopropylamine, N-methylmorpholine, N-methylpiperidine, 1,5-diazabicyclo[4.3.0]non-5-ene (DBN) or 1,8-diazabicyclo[5.4.0]undec-7-ene (DBU), or organometallic compounds such as butyllithium or phenyllithium. Pyridine is particularly preferred, where appropriate in the presence of catalytic amounts (approx. 10 mol %) of 4-N,N-dimethylaminopyridine or 4-pyrrolidinopyridine.

The base is employed in this case in an amount of from 1 to 10, preferably 1 to 3, mol per mol of the compound (Ia) or (II), where appropriate with the addition of catalytic amounts (approx. 10 mol %) of 4-N,N-dimethylaminopyridine or 4-pyrrolidonopyridine.

The acylation generally takes place in a temperature range from −30° C. to +100° C., preferably in a temperature range from 0° C. to +60° C.

The reactions can be carried out under atmospheric, elevated or reduced pressure (e.g. from 0.5 to 5 bar). They are generally carried out under atmospheric pressure.

Suitable solvents for process steps [C] (V)+(VI)→(VII) and [C] (VII)+(VIII)/(IX)→(X)/(XI) are all inert solvents. These include halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclohexane or petroleum fractions, nitroalkanes such as nitromethane, esters such as ethyl acetate, ketones such as acetone, heteroaromatic compounds such as pyridine, amides such as dimethylformamide, dialkyl sulfoxides such as dimethyl sulfoxide, or nitriles such as acetonitrile. It is likewise possible to employ mixtures of said solvents. Dichloromethane, tetrahydrofuran, acetonitrile, dimethylformamide or mixtures thereof are preferred.

Customary inorganic or organic bases are suitable as base for these process steps. These preferably include alkali metal or alkaline earth metal carbonates such as sodium, potassium or calcium carbonate, alkali metal hydrides such as sodium hydride, amides such as lithium bis(trimethylsilyl)amide or lithium diisopropylamide, organic amines such as pyridine, 4-N,N-dimethylaminopyridine, 4-pyrrolidinopyridine, triethylamine, ethyldiisopropylamine, N-methylmorpholine, N-methylpiperidine, 1,5-diazabicyclo[4.3.0]non-5-ene (DBN) or 1,8-diazabicyclo[5.4.0]undec-7-ene (DBU), or organometallic compounds such as butyllithium or phenyllithium. Triethylamine and ethyl diisopropylamine are particularly preferred.

The base is employed in this case in an amount of from 1 to 10, preferably 1 to 3, mol per mol of the compound (V) or (VI).

The reactions are generally carried out in a temperature range from −30° C. to +100° C., preferably in a temperature range from 0° C. to +60° C.

The reactions can be carried out under atmospheric, elevated or reduced pressure (e.g. from 0.5 to 5 bar). They are generally carried out under atmospheric pressure.

In the case of compounds of the formula (VII) in which Y² is imidazolide, the process step (VII)+(VIII/(IX)→(X)/(XI) is preferably carried out in the presence of equivalent amounts of methyl trifluoromethanesulfonate or methyl iodide as catalyst.

Synthesis of the compounds of the invention can be illustrated by the following formula schemes 1-4:

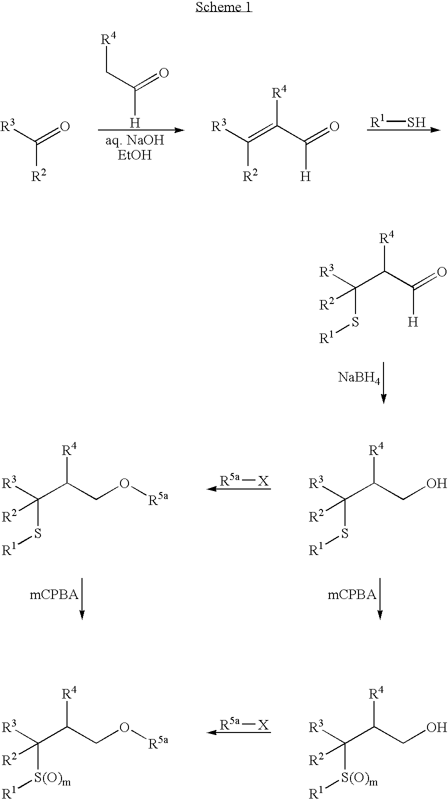

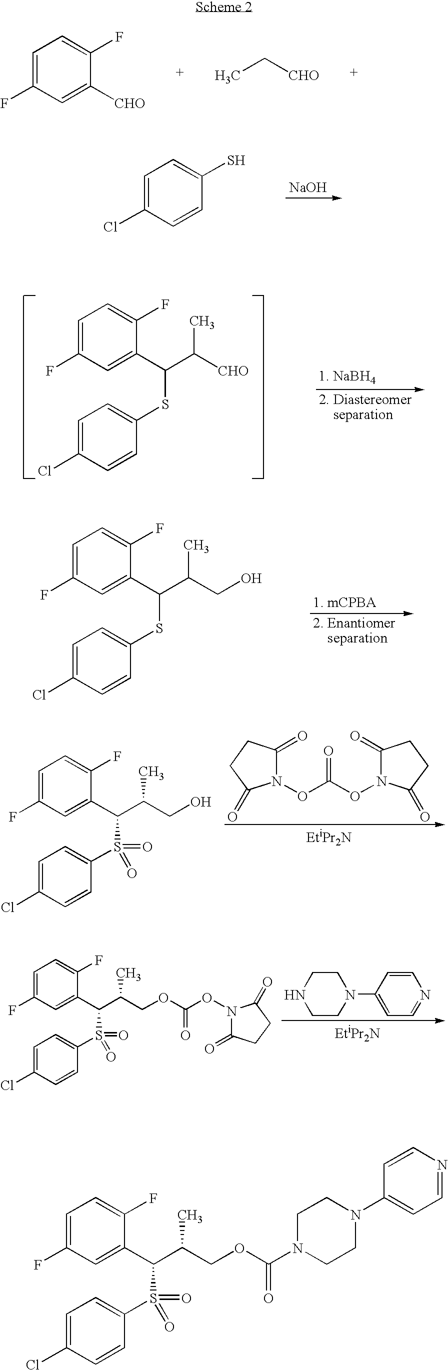

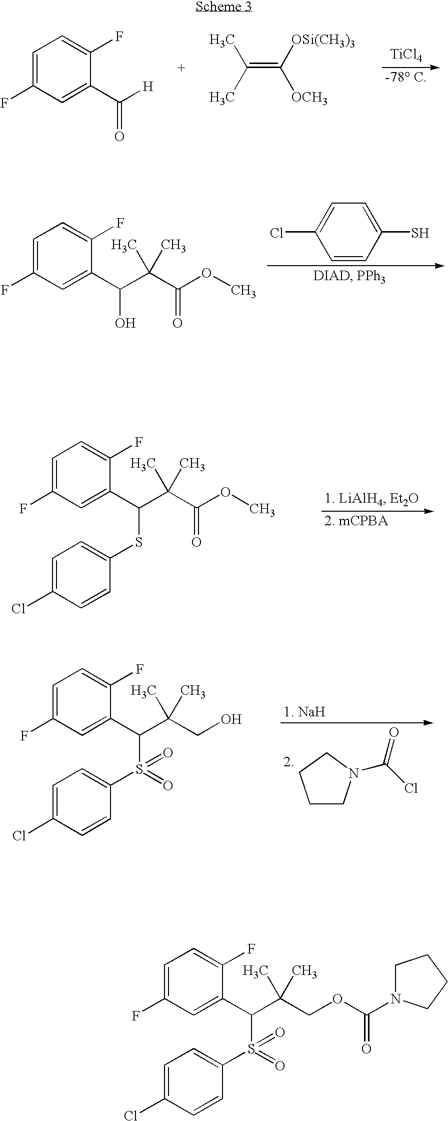

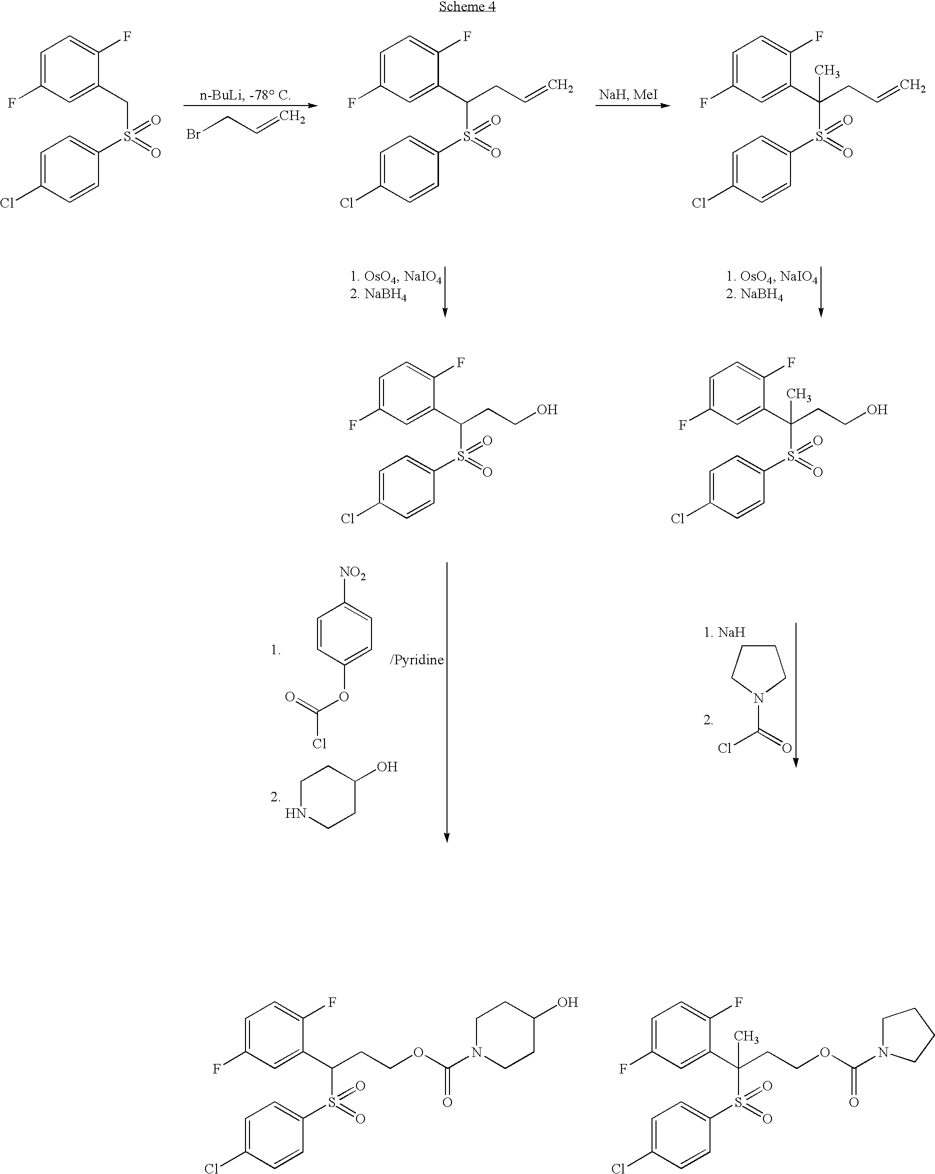

[Abbreviations: n-Bu=n-butyl, DIAD=diisopropyl azodicarboxylate, Et=ethyl, mCPBA=meta-chloroperbenzoic acid, Me=methyl, Ph=phenyl, ⁱPr=isopropyl].

The compounds of the invention show a valuable range of pharmacological and pharmacokinetic effects which could not have been predicted.

They are therefore suitable for use as medicaments for the treatment and/or prophylaxis of diseases in humans and animals.

The compounds of the invention inhibit γ-secretase.

The compounds of the invention can by reason of their pharmacological properties be employed alone or in combination with other active ingredients for the treatment and/or prevention of neurodegenerative diseases, especially of Alzheimer's disease.

The compounds of the invention can by reason of their pharmacological properties be employed alone or in combination with other medicaments for the treatment and/or prophylaxis of diseases which are associated with the increased formation, release, accumulation or deposition of amyloid peptides such as, for example, Aβ, especially for the treatment or prophylaxis of Alzheimer's disease and/or cognitive impairments associated therewith, which occur for example in situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, attention deficit hyperactivity disorder, Alzheimer's disease, Lewy body dementia, dementia with degeneration of the frontal lobes, including Pick's syndrome, Parkinson's disease, progressive nuclear palsy, dementia with corticobasal degeneration, amyotrophic lateral sclerosis (ALS), Huntington's disease, multiple sclerosis, thalamic degeneration, Creutzfeld-Jacob dementia, HIV dementia or schizophrenia with dementia.

The compounds of the invention can additionally be employed in combination with other medicaments which prevent the formation, release, accumulation or deposition of amyloid peptides in the brain. It is conceivable in this connection to combine with other medicaments which are inhibitors of beta- or gamma-secretase, medicaments which through their presence impede, delay or prevent the deposition of amyloid plaques. A further use of the compounds of the invention is possible in combination with a therapy which brings about an increased immune response to amyloid peptides.

The compounds of the invention can additionally be employed in combination with other medicaments which improve learning and memory.

The present invention further relates to medicaments which comprise at least one compound of the invention, preferably together with one or more pharmacologically acceptable excipients or carriers, and the use thereof for the aforementioned purposes.

The active ingredient may have systemic and/or local effects. For this purpose, it can be administered in a suitable manner such as, for example, by the oral, parenteral, pulmonary, nasal, sublingual, lingual, buccal, rectal, transdermal, conjunctival or otic route or as implant.

The active ingredient can be administered in suitable administration forms for the administration routes.

Administration forms suitable for oral administration are known ones which deliver the active ingredient rapidly and/or in a modified way, such as, for example, tablets (uncoated and coated tablets, e.g. tablets provided with coatings resistant to gastric juice, or film-coated tablets), capsules, sugar-coated tablets, granules, pellets, powders, emulsions, suspensions, solutions and aerosols.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous, or intraperitoneal). Administration forms suitable for parenteral administration include preparations for injection and infusion in the form of solutions, suspensions, emulsions, lyophilisates and sterile powders.

Examples suitable for the other administration routes are medicinal forms for inhalation (including powder inhalers, nebulizers), nasal drops/solutions, sprays; tablets or capsules for lingual, sublingual or buccal administration, suppositories, preparations for the ears and eyes, vaginal capsules, aqueous suspensions (lotions, shaking mixtures), lipophilic suspensions, ointments, creams, milk, pastes, dusting powders or implants.

The active ingredients can be converted in a manner known per se to the administration forms listed. This takes place with use of inert nontoxic, pharmaceutically suitable excipients. These include inter alia carriers (e.g. microcrystalline cellulose), solvents (e.g. liquid polyethylene glycols), emulsifiers (e.g. sodium dodecyl sulfate), dispersants (e.g. polyvinylpyrrolidone), synthetic and natural biopolymers (e.g. albumin), stabilizers (e.g. antioxidants such as ascorbic acid), colorants (e.g. inorganic pigments such as iron oxides) or masking tastes and/or odors.

It has generally proved advantageous for parenteral administration to administer amounts of about 0.001 to 10 mg/kg, preferably about 0.005 to 3 mg/kg, of body weight to achieve effective results. On oral administration, the amount is about 0.001 to 100 mg/kg, preferably about 0.005 to 30 mg/kg, of body weight.

It may nevertheless be necessary where appropriate to deviate from the amounts mentioned, in particular as a function of the body weight, administration route, individual response to the active ingredient, type of preparation and time or interval level at which administration takes place. Thus, in some cases, it may be sufficient to make do with less than the aforementioned minimum amount, whereas in other cases the upper limit mentioned must be exceeded. Where larger amounts are administered it may be advisable to divide these into a plurality of single doses over the day.

The percentage data in the following tests and examples are, unless indicated otherwise, percentages by weight; parts are parts by weight. Solvent ratios, dilution ratios and concentration data for liquid/liquid solutions are in each case based on volume.

Abbreviations:

| | |
|---|---|
| CI | chemical ionization (in MS) |
| DCI | direct chemical ionization (in MS) |
| DMF | N,N-dimethylformamide |
| DMSO | dimethyl sulfoxide |
| EI | electron impact ionization (in MS) |
| ESI | electrospray ionization (in MS) |
| HPLC | high pressure, high performance liquid chromatography |
| LC-MS | coupled liquid chromatography-mass spectroscopy |
| MS | mass spectroscopy |
| NMR | nuclear magnetic resonance spectroscopy |
| RT | room temperature |
| Rₜ | retention time (in HPLC) |
| THF | tetrahydrofuran |

Analytical Methods:
Method 1:

Instrument: HP 1100 with DAD detection; column: Kromasil RP-18, 60 mm×2 mm, 3.5 μm; eluent A=5 ml of HClO₄/l of H₂O, eluent B=acetonitrile; gradient: 0 min 2% B, 0.5 min 2% B, 4.5 min 90% B, 9 min 90% B; flow rate: 0.75 ml/min; temp.: 30° C.; UV detection: 210 nm.

Method 2:

MS instrument type: Micromass ZQ; HPLC instrument type: Waters Alliance 2790; column: Grom-Sil 120 ODS-4 HE 50 mm×2 mm, 3.0 μm; eluent B: acetonitrile+0.05% formic acid, eluent A: water+0.05% formic acid; gradient: 0.0 min 5% B 2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 3:

MS instrument type: Micromass ZQ; HPLC instrument type: Waters Alliance 2790; column: Uptisphere C 18, 50 mm×2 mm, 3.0 μm; eluent B: acetonitrile+0.05% formic acid, eluent A: water+0.05% formic acid; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 4:

Instrument: Micromass Quattro LCZ, with HPLC Agilent Serie 1100; column: Uptisphere HDO, 50 mm×2.0 mm, 3 μm; eluent A: 1 L of water+1 mL of 50% formic acid, eluent B: 1 L of acetonitrile+1 mL of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 5:

Instrument: Micromass Quattro LCZ, with HPLC Agilent Serie 1100; column: Grom-SIL120 ODS-4 HE, 50 mm×2.0 mm, 3 μm; eluent A: 1 L of water+1 mL of 50% formic acid, eluent B: 1 L of acetonitrile+1 mL of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 mm.

Method 6:

Instrument: Micromass Platform LCZ, with HPLC Agilent Serie 1100; column: Grom-SIL120 ODS-4 HE, 50 mm×2.0 mm, 3 μm; eluent A: 1 L of water+1 mL of 50% formic acid, eluent B: 1 L of acetonitrile+1 mL of 50% formic acid; gradient: 0.0 min 100% A→0.2 min 100% A→2.9 min 30% A→3.1 min 10% A→4.5 min 10% A; oven: 55° C.; flow rate: 0.8 ml/min; WV detection: 208-400 nm.

Method 7:

Instrument: Micromass Quattro LCZ, HP1100; column: Symmetry C18, 50 mm×2.1 mm, 3.5 μm; eluent A: water+0.05% formic acid, eluent B: acetonitrile+0.05% formic acid; gradient: 0.0 min 90% A→4.0 min 10% A→6.0 min 10% A; oven: 40° C.; flow rate: 0.5 ml/min; UV detection: 208-400 nm.

Method 8:

Instrument: Micromass Platform LCZ, HP1100; column: Symmetry C18, 50 mm×2.1 mm, 3.5 μm; eluent A: water+0.05% formic acid, eluent B: acetonitrile+0.05% formic acid; gradient: 0.0 min 90% A→4.0 min 10% A→6.0 min 10% A; oven: 40° C.; flow rate: 0.5 ml/min; WV detection: 208-400 nm.

Method 9:

MS instrument type: Micromass ZQ; HPLC instrument type: Waters Alliance 2790; column: Symmetry C18, 50 mm×2.1 mm, 3.5 μm; eluent B: acetonitrile+0.05% formic acid, eluent A: water+0.05% formic acid; gradient: 0.0 min 5% B→4.5 min 90% B→5.5 min 90% B; oven: 50° C.; flow rate: 1.0 ml/min; UV detection: 210 nm.

Starting Compounds:

### EXAMPLE 1A

### 3-[(4-Chlorophenyl)sulfanyl]-3-(2,5-difluorophenyl)-2-methyl-1-propanol

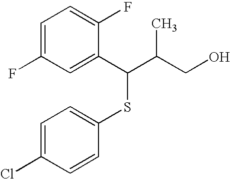

500 mg (3.45 mmol) of 2,5-difluorobenzaldehyde and 204 mg (3.45 mmol) of propionaldehyde are dissolved in 3 ml of ethanol, and 0.165 ml of 10% strength sodium hydroxide solution is added, and the mixture is stirred at RT for 24 h. Then 712 mg (4.83 mmol) of 4-chlorothiophenol are slowly added at RT. After a further 20 h, 130 mg (3.45 mmol) of sodium borohydride are added to the reaction solution, the amount being divided into two equally large portions and being added at an interval of 0.5 h. The mixture is stirred for 3.5 h. For workup, 10 ml of ice-water are added to the solution, and it is extracted three times with diethyl ether. The combined organic phases are dried over sodium sulfate and concentrated, and the residue is dried under high vacuum. The crude product is taken up in a little cyclohexane and chromatographed on silica gel (mobile phase cyclohexane/2 to 5% ethyl acetate). The product-containing fractions are combined, concentrated and dried under high vacuum. 542 mg (45% of theory) of a colorless oily product consisting of a mixture of the two diastereomers (content of each about 50%) are obtained.

MS (CI): m/z=346 [M+NH₄]⁺

¹H-NMR (200 MHz, DMSO-d₆): δ=7.4-7.0 (7H), 4.8-4.5 (2H), 3.65-3.1 (2H), 2.2-2.0 (1H), 1.1 (d, 3H, diastereomer A), 0.8 (d, 3H, diastereomer B).

### EXAMPLE 1A-1

Further fractionation by means of preparative HPLC (Kromasil 100 C18, mobile phase 30% by volume water/70% by volume acetonitrile) of the mixture of diastereomers of Example 1A affords, as component eluting first, pure diastereomer A (in racemic form).

MS (CI): m/z=346 [M+N]⁺

¹H-NMR (300 MHz, DMSO-d₆): δ=7.35-7.2 (m, 5H), 7.2-7.0 (m, 2H), 4.75 (t, 1H), 4.6 (d, 1H), 3.6 (t, 2H), 2.2-2.1 (m, 1H), 0.8 (d, 3H).

### EXAMPLE 1A-2

Further fractionation by means of preparative HPLC (Kromasil 100 C18, mobile phase 30% by volume water/70% by volume acetonitrile) of the mixture of diastereomers of Example 1A affords, as component eluting later, pure diastereomer B (in racemic form).

MS (CI): m/z=346 [M+NH₄]⁺

¹H-NMR (300 MHz, DMSO-d₆): δ=7.35-7.25 (m, 5H), 7.2-7.05 (m, 2H), 4.74.6 (m, 2H), 3.45-3.35 (m, 1H), 3.25-3.15 (m, 2H), 2.2-2.05 (m, 1H), 1.1 (d, 3H).

The following are obtained in an analogous manner:

### EXAMPLE 2A

### 2-[[(4-Chlorophenyl)sulfanyl](2,5-difluorophenyl)methyl]-1-butanol

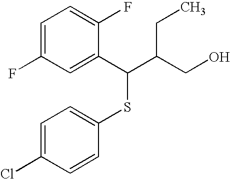

1.15 g (68% of theory) of a colorless oily product consisting of a mixture of the two diastereomers (approx. 60% diastereomer A, 40% diastereomer B) are obtained.

MS (CI): m/z=360 [M+NH₄]⁺

¹H-NMR (400 MHz, DMSO-d₆): δ=7.4-7.0 (7H), 4.75-4.6 (2H), 3.8-3.2 (2H), 2.0-1.1 (3H), 0.9 (t, 3H, diastereomer A), 0.8 (t, 3H, diastereomer B).

### EXAMPLE 3A

### 3-[(4-Chlorophenyl)sulfanyl]-3-(2,5-dichlorophenyl)-2-methyl-1-propanol

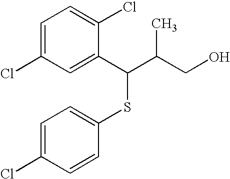

869 mg (50% of theory) of the product are obtained as a mixture of diastereomers (approx. 54% diastereomer A, 46% diastereomer B) as a colorless oil starting from 846 mg (4.74 mmol) of 2,5-dichlorobenzaldehyde.

MS (CI): m/z=378 [M+NH₄]⁺

¹H-NMR (200 MHz, DMSO-d₆): δ=7.6-7.15 (7H), 4.95-4.5 (2H), 3.7-3.2 (2H), 2.2-2.05 (1H), 1.0 (d, 3H, diastereomer A), 0.8 (d, 3H, diastereomer B).

### EXAMPLE 4A

### 3-[(4-Chlorophenyl)sulfanyl]-3-(2-fluoro-5-methylphenyl)-2-methyl-1-propanol

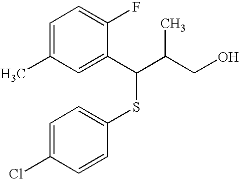

The product is obtained as a mixture of diastereomers (approx. 55% diastereomer A, 45% diastereomer B) as a colorless oil.

MS (CI): m/z=342 [M+NH₄]⁺

¹H-NMR (200 MHz, DMSO-d₆): δ=7.3-6.9 (7H), 4.74.5 (2H), 3.6-3.1 (2H), 2.2 (s, 3H), 2.15-2.05 (1H), 1.1 (d, 3H, diastereomer A), 0.8 (d, 3H, diastereomer B).

### EXAMPLE 5A

### 3-[(4-Chlorophenyl)sulfanyl]-3-(2,5-difluorophenyl)-2-methylpropyl N,N-diethylcarbamate

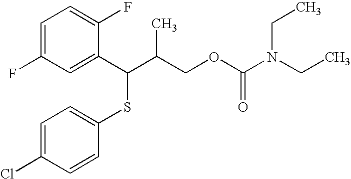

To a solution of 304 mg (0.74 mmol) of 3-[(4-chlorophenyl)sulfanyl]-3-(2,5-difluorophenyl)-2-methyl-1-propanol (Example 1A) in a mixture of 3.6 ml of tetrahydrofuran and 0.55 ml of acetonitrile are added firstly 62 mg (0.78 mmol) of pyridine and then, at 0° C., slowly 182 mg (0.85 mmol) of 4-nitrophenyl chloroformate. The mixture is stirred first at RT overnight and then at 55° C. for 4 h. At RT, a solution of 328 mg (4.44 mmol) of diethylamine in 5 ml of THF is added dropwise, and the mixture is stirred at RT for 3 h and then at 50° C. for 3 h. For workup, the solvent is removed in vacuo, and the residue is taken up in dichloromethane and washed with water. The organic phase is dried over sodium sulfate and concentrated. The crude product is first chromatographed on silica gel (mobile phase: cyclohexane/1 to 5% ethyl acetate) and then purified by HPLC. 122 mg (38% of theory) of a colorless oily product consisting of a mixture of the two diastereomers (approx. 55% diastereomer A, 45% diastereomer B) are obtained.

MS (ESI): m/z=428 [M+NH₄]⁺

¹H-NMR (300 MHz, DMSO-d₆): δ=7.4-7.0 (7H), 4.6-4.5 (1H), 4.2-3.7 (2H), 3.25-3.1 (4H), 2.4 (1H), 1.1 (d, 3H, diastereomer A), 1.1-0.95 (6H), 0.85 (d, 3H, diastereomer B).

The following is obtained in an analogous manner:

### EXAMPLE 6A

### 3-[(4-Chlorophenyl)sulfanyl]-3-(2,5-difluorophenyl)-2-methylpropyl 1-pyrrolidinecarboxylate

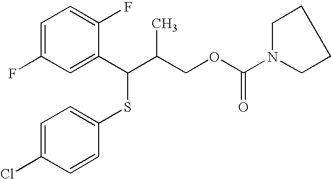

540 mg of a colorless oily product (87% of theory) consisting of a mixture of the two diastereomers (approx. 60% diastereomer A, 40% diastereomer B) are obtained.

MS (ESI): m/z=426 [M+H]⁺

¹H-NMR (300 MHz, DMSO-d₆): δ=7.4-7.0 (7H), 4.6-4.5 (1H), 4.2-3.7 (2H), 3.25-3.1 (4H), 2.55-2.35 (1H), 1.8 (4H), 1.15 (d, 3H, diastereomer A), 0.9 (d, 3H, diastereomer B).

### EXAMPLE 7A

### 3-[(4-Chlorophenyl)sulfanyl]-3-(2,5-difluorophenyl)-2-methylpropyl benzoate

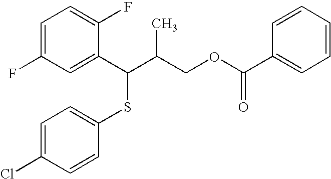

55 mg (0.39 mmol) of benzoyl chloride are added to a solution of 86 mg (0.26 mmol) of 3-[(4-chlorophenyl)sulfanyl]-3-(2,5-difluorophenyl)-2-methyl-1-propanol (Example 1A) in 0.5 ml of pyridine at RT, and the mixture is stirred for 2 hours. The solution is concentrated in vacuo, and the residue is taken up in dichloromethane and washed with 2% strength sodium bicarbonate solution. The organic phase is dried over sodium sulfate, concentrated and purified by preparative HPLC. 78% (69% of theory) of the product are obtained as a mixture of diastereomers (approx. 50% diastereomer A, 50% diastereomer B).

MS (CI): m/z=450 [M+NH₄]⁺

¹H-NMR (300 MHz, DMSO-d₆): δ=8.0-7.0 (12H), 4.75-4.65 (1H), 4.55-4.0 (2H), 2.7-2.5 (1H), 1.3 (d, 3H, diastereomer A), 1.0 (d, 3H, diastereomer B).

### EXAMPLE 8A

### 4-{[1-(2,5-Difluorophenyl)-3-hydroxy-2-methylpropyl]sulfonyl}benzonitrile

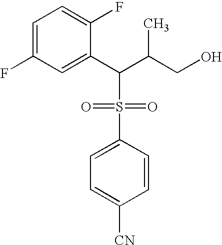

The compound is prepared in analogy to the method of Example 1A and of Example 1 [the p-cyanothiophenol used as starting material is prepared in accordance with J. Org. Chem. 54, 4458-4462 (1998)]. The final product obtained after oxidation is employed without further purification in the subsequent reaction.

HPLC (method 1): Rₜ=4.23 and 4.30 min. (mixture of diastereomers)

MS (ESI pos.): m/z=352 [M+H]⁺.

### EXAMPLE 9A

### N-Ethyl-1-piperazinecarboxamide trifluoroacetate

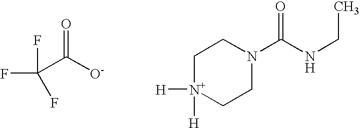

800 mg (0.80 mmol) of p-nitrophenyl carbonate-Wang polystyrene resin (from Novabiochem) are mixed with a solution of 0.3 ml (4.00 mmol) of piperazine in 15 ml of N,N-dimethylformamide, and the mixture is shaken at room temperature for 16 h. The resin is filtered off and washed several times with N,N-dimethylformamide, methanol and dichloromethane. A solution of 0.32 ml (4.00 mmol) of ethyl isocyanate in 5 ml of THF is then added, and 10 mg (0.08 mmol) of N,N-dimethylaminopyridine are added. The mixture is shaken at room temperature for 16 h, and the resin is filtered off and washed several times with N,N-dimethylformamide, methanol and dichloromethane. The product is eliminated from the support resin by treatment with 20 ml of trifluoroacetic acid/dichloromethane (1:1 v/v) at room temperature for 1 h, and the polymer is filtered off and the filtrate is concentrated in vacuo. The product is pure enough for further reactions.

MS (ESI pos.): m/z=158 [M+H]⁺.

### EXAMPLE 10A

### 3-(2,5-Difluorophenyl)-2-methyl-3-{[4-(trifluoromethyl)phenyl]sulfonyl}-1-propanol

Stage a):

### 3-(2,5-Difluorophenyl)-2-methyl-2-propenal

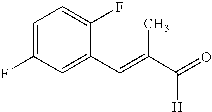

75 g (528 mmol) of 2,5-difluorobenzaldehyde and 30.6 g (528 mmol) of propional are dissolved in 450 ml of ethanol and, while cooling in ice, 25 ml (62.5 mmol) of 2.5 M sodium hydroxide solution are added, and the mixture is stirred at room temperature overnight. It is then poured into ice-water/hydrochloric acid, taken up in ethyl acetate, washed with water and concentrated. Subsequent chromatography (silica gel, mobile phase: petroleum ether) affords 55.2 g (55% of theory) of the title compound.

MS (EI): m/z=182 [M]⁺

¹H-NMR (300 MHz, CDCl₃): δ=9.6 (s, 1H), 7.35 (s, 1H), 7.3-7.2 (m, 1H), 7.15-7.05 (m, 2H), 2.05 (s, 3H).

Stage b):

### 3-(2,5-Difluorophenyl)-2-methyl-3-{[4-(trifluoromethyl)phenyl]sulfonyl}-1-propanol

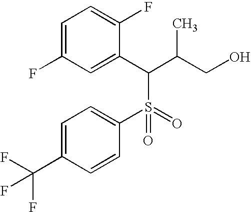

0.22 ml (0.44 mmol) of 2 M sodium hydroxide solution and 900 mg (5.05 mmol) of 4-trifluoromethylthiophenol are added to a solution of 657 mg (3.61 mmol) of 3-(2,5-difluorophenyl)-2-methyl-2-propenal in 5 ml of ethanol at 0° C. and stirred at room temperature overnight. The mixture is then cooled in an ice bath and 150 mg (3.97 mmol) of sodium borohydride are slowly added in portions, and the mixture is stirred at room temperature for 9 h. It is diluted with 15 ml of dichloromethane and cooled to 0° C., and 3.56 g (70% purity; 14.4 mmol) of 3-chloroperbenzoic acid are added in two portions at an interval of one hour and stirred at room temperature overnight. Addition of saturated sodium thiosulfate solution is followed by extraction with dichloromethane. The organic phase is washed with saturated sodium bicarbonate solution, dried over magnesium sulfate and concentrated. Purification by preparative HPLC (RP18 column, eluent acetonitrile/water) affords 907 mg (64% of theory) of the title compound as a mixture of diastereomers.

LC/MS (method 2): Rₜ=3.82 min, m/z=417 [M+Na]⁺.

### EXAMPLE 10A-1

### (2R,3S)-3-(2,5-Difluorophenyl)-2-methyl-3-{[4-(trifluoromethyl)phenyl]sulfonyl}-1-propanol

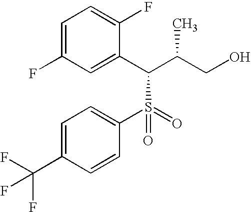

Further fractionation by means of preparative HPLC (Kromasil 60 Si, mobile phase 90% by volume isohexane/10% by volume isopropanol) of the mixture of diastereomers of Example 10A affords, as component eluting later, pure diastereomer B in racemic form. Subsequently, further fractionation of the racemate of diastereomer B by means of preparative HPLC on a chiral phase (Daicel Chiralpak AD, mobile phase ethanol) affords, as component eluting later, the title compound as pure enantiomer.

MS (ESI): m/z=417 [M+Na]⁺

¹H-NMR (200 MHz, DMSO-d₆): δ=7.85 (d, 2H), 7.75 (d, 2H), 7.4-7.3 (m, 1H), 7.25-7.1 (m, 1H), 7.05-6.9 (m, 1H), 4.8-4.65 (m, 2H), 3.35-3.25 (m, 1H), 3.1-3.0 (m, 1H), 2.75-2.65 (m, 1H), 1.4 (d, 3H).

The following starting compounds are prepared as described in the reference detailed in each case:

### EXAMPLE 11A

### 1-(3-Chlorophenyl)-2-piperazinone hydrochloride

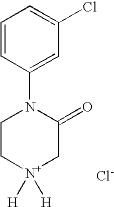

The title compound is obtained in accordance with Tetrahedron Lett. 39, 7459-7562 (1998).

### EXAMPLE 12A

### 1-(3-Trifluoromethoxyphenyl)-2-piperazinone hydrochloride

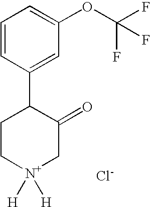

The title compound is obtained in an analogous manner to Example 11A.

### EXAMPLE 13A

### 5-Fluoro-2-methylbenzaldehyde

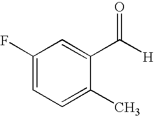

The title compound is obtained in accordance with J. Am. Chem. Soc. 90, 6712-6717 (1968).

### EXAMPLE 14A

### N-[2-(1-piperazinyl)phenyl]methanesulfonamide

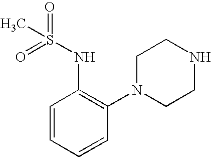

The title compound is obtained in accordance with Bioorg. Med. Chem. Lett. 8, 1851-1856 (1998).

### EXAMPLE 15A

### 4-Ethylpiperidine

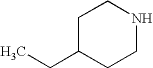

The title compound is obtained in accordance with J. Heterocycl. Chem. 13, 955-960 (1976).

### EXAMPLE 16A

### 4,4-Dimethylpiperidine

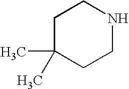

The title compound is obtained in accordance with J. Med. Chem. 8, 766-776 (1965).

### EXAMPLE 17A

### N-Methyl-2-butanamine

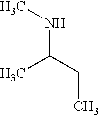

The title compound is obtained in accordance with J. Am. Chem. Soc. 77, 3061-3067 (1955).

### EXEMPLARY EMBODIMENTS

### Example 1

### 3-[(4-Chlorophenyl)sulfonyl]-3-(2,5-difluorophenyl)-2-methyl 1-propanol

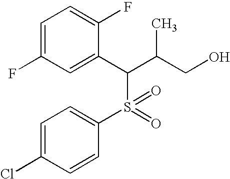

3.75 g (10.94 mmol) of 3-[(4-chlorophenyl)sulfanyl]-3-(2,5-difluorophenyl)-2-methyl-1-propanol (Example 1A) are dissolved in 60 ml of methylene chloride and, at RT, 5.40 g (70% purity; 21.9 mmol) of meta-chloroperbenzoic acid are slowly added. After two hours, 200 ml of 2.5% strength sodium bicarbonate solution are added to the reaction solution, the phases are separated, and the aqueous phase is back-extracted three times with methylene chloride. The combined organic phases are dried over sodium sulfate, concentrated and chromatographed on silica gel (mobile phase: cyclohexane/2 to 20% ethyl acetate). 3.7 g (90% pure by HPLC, 84% of theory) of the product are obtained as a mixture of diastereomers (approx. 45% diastereomer A, 55% diastereomer B) as a colorless oil. 100% pure product can be obtained by further chromatography.

MS (CI): m/z=378 [M+NH₄]⁺

¹H-NMR (200 MHz, DMSO-d₆): δ=7.6 (s, 2H), 7.5 (s, 2H), 7.4-7.0 (3H), 4.95-4.6 (2H), 3.65-3.0 (2H), 2.7-2.5 (1H), 1.4 (d, 3H, diastereomer A), 0.95 (d, 3H, diastereomer B).

### Example 1-1

### rac-(2R,3R)-3-[(4-Chlorophenyl)sulfonyl]-3-(2,5-difluorophenyl)-2-methyl-1-propanol

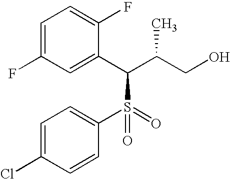

The title compound is obtained in an analogous manner from Example 1A-1.

MS (CI): m/z=378 [M+NH₄]⁺

¹H-NMR (200 MHz, DMSO-d₆): δ=7.6 (s, 4H), 7.45-7.35 (m, 1H), 7.3-7.05 (m, 2H), 4.95 (d, 1H), 4.85 (t, 1H), 3.6-3.45 (m, 1H), 3.4-3.3 (m, 1H), 2.8-2.65 (m, 1H), 0.95 (d, 3H).

### Example 1-2

### rac-(2R,3S)-3-[(4-Chlorophenyl)sulfonyl]-3-(2,5-difluorophenyl)-2-methyl-1-propanol

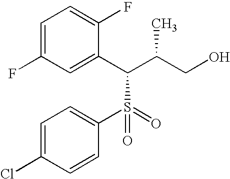

The title compound is obtained in an analogous manner from Example 1A-2.

MS (CI): m/z=378 [M+NH₄]⁺

¹H-NMR (200 MHz, DMSO-d₆): δ=7.55 (s, 4H), 7.4-7.3 (m, 1H), 7.25-7.1 (m, 1H), 7.1-6.95 (m, 1H), 4.75-4.65 (m, 2H), 3.35-3.25 (m, 1H), 3.1-2.95 (m, 1H), 2.75-2.6 (m, 1H), 1.4 (d, 3H).

### Example 1-3

Pure enantiomer 1 can be obtained as faster-eluting component from the racemate of Example 1-1 by further fractionation by means of preparative HPLC on a chiral phase (Daicel Chiralcel OD, mobile phase 75% by volume isohexane/25% by volume isopropanol).

### Example 1-4

Pure enantiomer 2, which is complementary to Example 1-3, can be obtained as component eluting later from the racemate of Example 1-1 by further fractionation by means of preparative HPLC on a chiral phase (Daicel Chiralcel OD, mobile phase 75% by volume isohexane/25% by volume isopropanol).

### Example 1-5

### (2S,3R)-3-[(4-Chlorophenyl)sulfonyl]-3-(2,5-difluorophenyl)-2-methyl-1-propanol

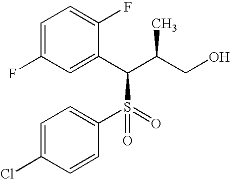

Pure enantiomer 3 can be obtained as faster-eluting component from the racemate of Example 1-2 by further fractionation by means of preparative HPLC on a chiral phase (Daicel Chiralpak AD, mobile phase ethanol).

### Example 1-6

### (2R,3S)-3-[(4-Chlorophenyl)sulfonyl]-3-(2,5-difluorophenyl)-2-methyl-1-propanol

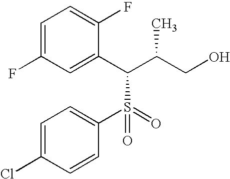

Pure enantiomer 4, which is complementary to Example 1-5, can be obtained as component eluting later from the racemate of Example 1-2 by further fractionation by means of preparative HPLC on a chiral phase (Daicel Chiralpak AD, mobile phase ethanol), and its absolute configuration was determined by single-crystal X-ray structure analysis.

The following are obtained in an analogous manner:

### Example 2

### 2-[[(4-Chlorophenyl)sulfonyl](2,5-difluorophenyl)methyl]-1-butanol

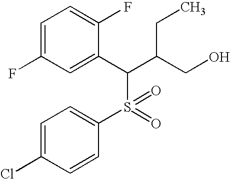

Oxidation of 1.14 g of 2-[[(4-chlorophenyl)sulfanyl](2,5-difluorophenyl)methyl]-1-butanol (Example 2A) results in 915 mg (77% of theory) of the product as a mixture of diastereomers (approx. 60% diastereomer A, 40% diastereomer B) as a colorless oil.

MS (CI): m/z=392 [M+NH₄]⁺

¹H-NMR (300 MHz, DMSO-d₆): δ=7.6-7.5 (4H), 7.4-6.95 (3H), 5.0-4.5 (2H), 3.85-3.0 (2H), 2.6-2.4 (1H), 2.0-1.0 (2H), 0.95 (t, 3H, diastereomer A), 0.85 (t, 3H, diastereomer B).

### Example 3

### 3-[(4-Chlorophenyl)sulfonyl]-3-(2,5-dichlorophenyl)-2-methyl-1-propanol

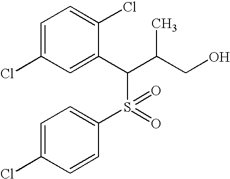

Oxidation of 855 mg (80% pure, 1.89 mmol) of 3-[(4-chlorophenyl)sulfanyl]-3-(2,5-dichlorophenyl)-2-methyl-1-propanol (Example 3A) results in 550 mg (74% of theory) of the product as a mixture of diastereomers (approx. 60% diastereomer A, 40% diastereomer B) as a colorless oil.

MS (CI): m/z=410 [M+NH₄]⁺

¹H-NMR (200 MHz, DMSO-d₆): δ=7.7-7.25 (7H), 5.15-4.65 (2H), 3.7-2.95 (2H), 2.85-2.5 (1H), 1.4 (d, 3H, diastereomer A), 0.9 (d, 3H, diastereomer B).

### Example 4

### 3-[(4-Chlorophenyl)sulfonyl]-3-(2-fluoro-5-methylphenyl)-2-methyl-1-propanol

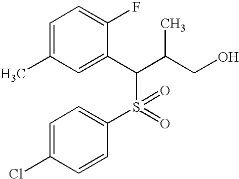

Oxidation of 740 mg (80% pure, 1.89 mmol) of 3-[(4-chlorophenyl)sulfanyl]-3-(2-fluoro-5-methylphenyl)-2-methyl-1-propanol (Example 4A) results in 550 mg (70% of theory) of the product as a mixture of diastereomers (approx. 57% diastereomer A, 43% diastereomer B) as a colorless oil.

MS (CI): m/z=374 [M+NH₄]⁺

¹H-NMR (300 MHz, DMSO-d₆): δ=7.6-6.7 (7H), 4.94.6 (2H), 3.55-3.0 (2H), 2.75-2.55 (1H), 2.35-2.25 (3H), 1.4 (d, 3H, diastereomer A), 0.95 (d, 3H, diastereomer B).

### Example 5

### 3-[(4-Chlorophenyl)sulfinyl]-3-(2,5-difluorophenyl)-2-methylpropyl N,N-diethylcarbamate

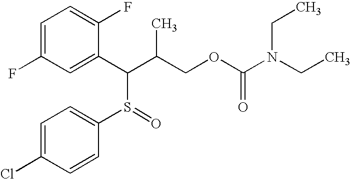

100 mg (0.23 mmol) of 3-[(4-chlorophenyl)sulfanyl]-3-(2,5-difluorophenyl)-2-methylpropyl N,N-diethylcarbamate (Example 5A) are dissolved in 1.5 ml of methylene chloride and, at 0° C., 58 mg (70% pure; 0.23 mmol) of meta-chloroperbenzoic acid are slowly added. After 30 minutes, 5 ml of 2.5% strength sodium bicarbonate solution are added to the reaction solution, the phases are separated, and the aqueous phase is back-extracted three times with methylene chloride. The combined organic phases are dried over sodium sulfate, concentrated and purified by preparative HPLC. All the fractions which have the correct molecular mass according to LC/MS and contain one of the product isomers are combined. 82 mg (79% of theory) of the product are obtained as a mixture of the four diastereomers as a colorless oil.

MS (CD): m/z=461 [M+NH₄]⁺

¹H-NMR (300 MHz, DMSO-d₆): δ 7.65-6.8 (7H), 4.6-4.5 (1H), 5.0-3.5 (3H), 3.4-3.0 (4H), 2.9-2.6 (1H), 1.6-0.8 (9H).

### Example 6

### 3-[(4-Chlorophenyl)sulfonyl]-3-(2,5-difluorophenyl)-2-methylpropyl N,N-diethylcarbamate

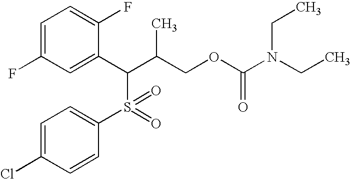

In analogy to the oxidation procedure in Example 1, starting from 800 mg (1.87 mmol) of 3-[(4-chlorophenyl)sulfanyl]-3-(2,5-difluorophenyl)-2-methylpropyl N,N-diethylcarbamate (Example 5A), a total of 676 mg (77% of theory) of the product are obtained as a mixture of diastereomers (approx. 54% diastereomer A, 46% diastereomer B) as a colorless oil.

MS (ESI): m/z=460 [M+H]⁺

¹H-NMR (300 MHz, DMSO-d₆): δ=7.7-7.5 (4H), 7.5-6.9 (3H), 4.9-4.65 (1H), 4.2-3.55 (2H), 3.3-2.8 (5H), 1.45 (d, 3H, diastereomer A), 1.15-0.9 (6H diastereomer A and B+3H diastereomer B).

### Example 7

### 3-[(4-Chlorophenyl)sulfonyl]-3-(2,5-difluorophenyl)-2-methylpropyl benzoate

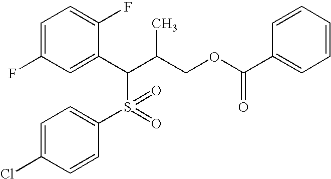

In analogy to the oxidation procedure in Example 1, starting from 65 mg (0.15 mmol) of 3-[(4-chlorophenyl)sulfanyl]-3-(2,5-difluorophenyl)-2-methylpropyl benzoate (Example 7A), a total of 59 mg (84% of theory) of the product are obtained as a mixture of diastereomers (approx. 46% diastereomer A, 54% diastereomer B) as a colorless oil.

MS (CI): m/z=450 [M+NH₄]⁺

¹H-NMR (300 MHz, DMSO-d₆): δ=8.0-6.9 (12H), 5.1-4.9 (1H), 4.5-3.9 (2H), 3.2-3.05 (1H), 1.55 (d, 3H, diastereomer A), 1.1 (d, 3H, diastereomer B).

### Example 8

### 3-[(4-Chlorophenyl)sulfonyl]-3-(2,5-difluorophenyl)-2-methylpropyl 4-morpholinecarboxylate

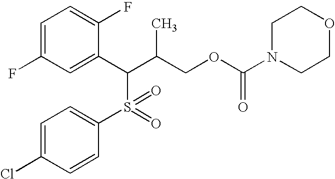

In analogy to the method in Example 5A, starting from 70 mg (0.19 mmol) of 3-[(4-chlorophenyl)sulfonyl]-3-(2,5-difluorophenyl)-2-methyl-1-propanol (Example 1), a total of 26 mg (28% of theory) of the product are obtained, after purification by preparative HPLC, as a mixture of diastereomers (approx. 40% diastereomer A, 60% diastereomer B) as a colorless oil.

MS (ESI): m/z=474 [M+H]⁺

¹H-NMR (300 MHz, CD₃OD): δ=7.65-7.3 (4H), 7.2-6.8 (3H), 4.9-4.7 (1H), 4.35-3.8 (2H), 3.7-3.55 (4H), 3.45-3.3 (4H), 3.15-3.0 (1H), 1.5 (d, 3H, diastereomer A), 1.1 (d, 3H, diastereomer B).

### Example 9

### 3-[(4-Chlorophenyl)sulfonyl]-3-(2,5-difluorophenyl)-2-methylpropyl 4-methyl-1-piperazinecarboxylate formate salt

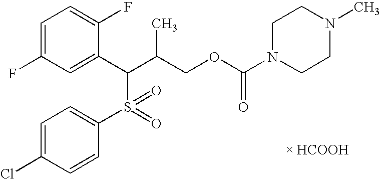

In analogy to the method in Example 5A, starting from 70 mg (0.19 mmol) of 3-[(4-chlorophenyl)sulfonyl]-3-(2,5-difluorophenyl)-2-methyl-1-propanol (Example 1), a total of 20 mg (19% of theory) of the product are obtained, after purification by preparative HPLC, as a mixture of diastereomers (approx. 50% diastereomer A, 50% diastereomer B) as formic acid salt (from the HPLC).

MS (ESI): m/z=487 [M+H]⁺

¹H-NMR (300 MHz, CD₃OD): δ=8.2 (1H, formate), 7.65-7.3 (4H), 7.2-6.8 (3H), 4.9-4.7 (1H), 4.35-3.8 (2H), 3.6-3.5 (4H), 3.15-3.0 (1H), 2.9-2.7 (4H), 2.6 (3H), 1.5 (d, 3H, diastereomer A), 1.1 (d, 3H, diastereomer B).

### Example 10

### 3-[(4-Chlorophenyl)sulfonyl]-3-(2,5-difluorophenyl)-2-methylpropyl 1-pyrrolidinecarboxylate

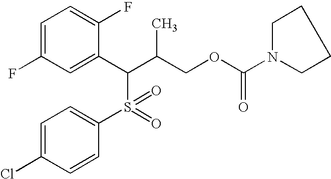

In analogy to the oxidation method in Example 1, starting from 85 mg (0.2 mmol) of 3-[(4-chlorophenyl)sulfanyl]-3-(2,5-difluorophenyl)-2-methylpropyl 1-pyrrolidinecarboxylate (Example 6A), a total of 72 mg (79% of theory) of the product are obtained, after purification by preparative HPLC, as a mixture of diastereomers (approx. 43% diastereomer A, 47% diastereomer B) as a colorless oil.

MS (ESI): m/z=458 [M+H]⁺

¹H-NMR (300 MHz, DMSO-d₆): δ=7.7-6.9 (7H), 4.9-4.7 (1H), 4.15-3.6 (2H), 3.3-3.1 (4H), 3.05-2.9 (1H), 1.9-1.7 (4H), 1.45 (d, 3H, diastereomer A), 1.0 (d, 3H, diastereomer B).

### Example 10-1

Further fractionation of the mixture of diastereomers of Example 10 by means of preparative HPLC (Kromasil 100 C18, mobile phase 50% by volume acetonitrile/50% by volume water) affords, as component eluting first, the pure diastereomer A (in racemic form).

¹H-NMR (300 MHz, DMSO-d₆): δ=7.6 (m, 4H), 7.35 (m, 1H), 7.15 (m, 1H), 7.0 (m, 1H), 4.7 (d, J=9 Hz, 1H), 3.95 (dd, 1H), 3.65 (dd, 1H), 3.3-3.1 (4H), 3.0 (m, 1H), 1.9-1.7 (4H), 1.45 (d, 3H).

### Example 10-2

Further fractionation of the mixture of diastereomers of Example 10 by means of preparative HPLC (Kromasil 100 C18, mobile phase 50% by volume acetonitrile/50% by volume water) affords, as component eluting later, the pure diastereomer B (in racemic form).

¹H-NMR (400 MHz, DMSO-d₆): δ=7.65 (m, 4H), 7.4 (m, 1H), 7.25 (m, 1H), 7.15 (m, 1H), 4.85 (d, J=7 Hz, 1H), 4.1-3.95 (2H), 3.2-3.1 (4H), 2.95 (m, 1H), 1.85-1.7 (4H), 1.0 (d, 3H).

### Example 10-3

The faster-eluting enantiomer 1 can be obtained from diastereomer A of Example 10-1 by further fractionation by means of preparative HPLC on a chiral phase (Daicel Chiralpak AS, mobile phase 87% isohexane/13% ethanol).

### Example 10-4

Enantiomer 2, which is complementary to Example 10-3 and elutes later, can be obtained from diastereomer A of Example 10-1 by further fractionation by means of preparative HPLC on a chiral phase (Daicel Chiralpak AS, mobile phase 87% isohexane/13% ethanol).

### Example 10-5

The faster-eluting enantiomer 3 can be obtained from diastereomer B of Example 10-2 by further fractionation by means of preparative HPLC on a chiral phase (Daicel Chiralpak AS, mobile phase 87% isohexane/13% ethanol).

### Example 10-6

Enantiomer 4, which is complementary to Example 10-5 and elutes later, can be obtained from diastereomer B of Example 10-2 by further fractionation by means of preparative HPLC on a chiral phase (Daicel Chiralpak AS, mobile phase 87% isohexane/13% ethanol).

### Example 11

### (2R,3S)-3-[(4-Chlorophenyl)sulfonyl]-3-(2,5-difluorophenyl)-2-methylpropyl 4-(4-pyridinyl)-1-piperazinecarboxylate

Stage a):

### 1-[({[(2R,3S)-3-[(4-Chlorophenyl)sulfonyl]-3-(2,5-difluorophenyl)-2-methylpropyl]-oxy}carbonyl)oxy]-2,5-pyrrolidinedione

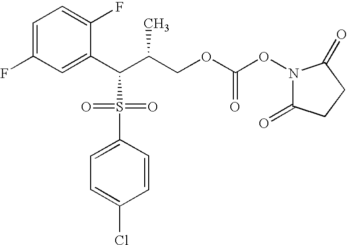

1.45 ml (8.32 mmol) of diisopropylethylamine and 1.06 g (4.16 mmol) of N,N′-disuccidinyl carbonate are added to a solution of 1.00 g (2.77 mmol) of (2R,3S)-3-[(4-chlorophenyl)sulfonyl]-3-(2,5-difluorophenyl)-2-methyl-1-propanol (Example 1-6) in 7.5 ml of acetonitrile. The mixture is stirred at room temperature for 3 h, then diluted with ethyl acetate and washed twice with saturated sodium bicarbonate solution. The combined aqueous phases are extracted with ethyl acetate, and the organic phases obtained in this way are combined, dried over sodium sulfate and freed of solvent in vacuo. The resulting product is pure enough for further reactions. 1.45 g (75% of theory) of a cream-colored solid are obtained.

LC/MS (method 2): Rₜ=3.67 min, m/z=502 [M+H]⁺.

Stage b):

### (2R,3S)-3-[(4-Chlorophenyl)sulfonyl]-3-(2,5-difluorophenyl)-2-methylpropyl 4-(4-pyridinyl)-1-piperazinecarboxylate

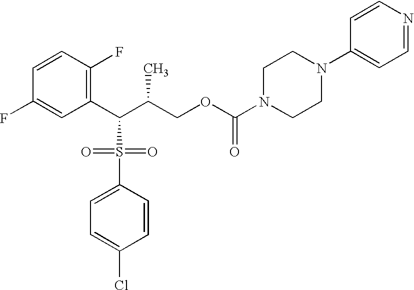

A solution of 20 mg (0.12 mmol) of 1-(4-pyridyl)piperazine in 1 ml of dichloromethane is added to a solution of 50 mg (0.10 mmol) of 1-[({[(2R,3S)-3-[(4-chlorophenyl)sulfonyl]-3-(2,5-difluorophenyl)-2-methylpropyl]oxy}carbonyl)oxy]-2,5-pyrrolidinedione and 0.78 ml (0.45 mmol) of diisopropylethylamine in 1 ml of dichloromethane. The mixture is stirred at room temperature for 2 h and then concentrated in vacuo. The crude mixture is separated by preparative HPLC. 25 mg (46% of theory) of a colorless oil are obtained.

¹H-NMR (200 MHz, CDCl₃): δ=8.51-8.22 (m, 3H), 7.55-7.22 (m, 4H), 7.02-6.88 (m, 1H), 6.83-6.62 (m, 3H), 4.53 (d, 1H), 4.13 (dd, 1H), 3.85 (dd, 1H), 3.72-3.41 (br, 8H), 3.15-2.92 (m, 1H), 1.51 (d, 1H).

LC/MS (method 3): Rₜ=2.85 min, m/z=550 [M+H]⁺.

### Example 12

### (2R,3S)-3-[(4-Chlorophenyl)sulfonyl]-3-(2,5-difluorophenyl)-2-methylpropyl 3-oxo-1-piperazinecarboxylate

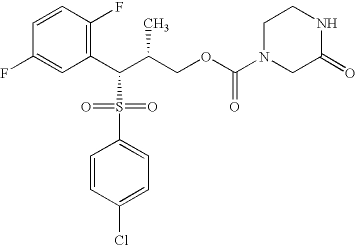

The compound is obtained in analogy to Example 11 above.

¹H-NMR (400 MHz, CDCl₃): δ=7.50 (d, 2H), 7.42-7.24 (m, 3H), 6.98-6.87 (m, 1H), 6.78-6.63 (m, 1H), 6.21-6.07 (br, 1H), 4.53 (d, 1H), 4.28-3.92 (m, 3H), 3.82 (dd, 1H), 3.70-3.53 (br, 2H), 3.45-3.81 (br, 2H), 3.07-2.92 (m, 1H), 1.58 (d, 1H).

LC/MS (method 3): Rₜ=3.37 min, m/z=487 [M+H]⁺.

### Example 13

### (2R,3S)-3-[(4-Chlorophenyl)sulfonyl]-3-(2,5-difluorophenyl)-2-methylpropyl tert-butylcarbamate

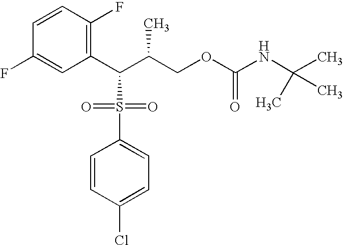

The compound is obtained in analogy to Example 11 above.

¹H-NMR (300 MHz, CDCl₃): δ=7.48 (d, 2H), 7.48-7.24 (m, 3H), 6.95-6.85 (m, 1H), 6.72-6.63 (m, 1H), 4.60-4.50 (m, 2H), 3.98-3.88 (m, 1H), 3.74 (dd, 1H), 2.98-2.83 (m, 1H), 1.52 (d, 1H), 1.28 (s, 9H).

LC/MS (method 3): Rₜ=4.27 min, m/z=460 [M+H]⁺.

### Example 14

### (2R,3S)-3-[(4-Chlorophenyl)sulfonyl]-3-(2,5-difluorophenyl)-2-methylpropyl 4-propionyl-1-piperazinecarboxylate

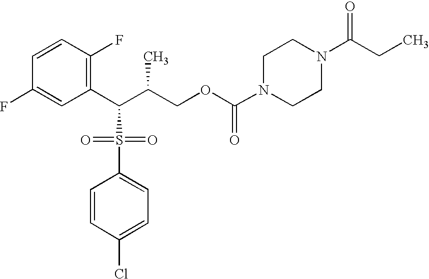

The compound is obtained in analogy to Example 11 above.

¹H-NMR (400 MHz, CDCl₃): δ=7.49 (d, 2H), 7.42-7.27 (m, 3H), 6.98-6.88 (m, 1H), 6.77-6.67 (m, 1H), 4.53 (d, 1H), 4.10 (dd, 1H), 3.82 (dd, 1H), 3.68-3.52 (br, 4H), 3.51-3.23 (br, 4H), 3.07-2.92 (m, 1H), 2.47 (q, 2H), 1.58 (d, 1H), 1.17 (t, 3H).

LC/MS (method 3): Rₜ=3.73 min, m/z=5.29 [M+H]⁺.

The trifluoroacetate of 1-propionylpiperazine is employed in this case and is obtained as follows:

1.00 g (1.00 mmol) of p-nitrophenyl carbonate-Wang polystyrene resin (from Novabiochem) is mixed with a solution of 0.39 ml (5.00 mmol) of piperazine in 20 ml of N,N-dimethylformamide, and the mixture is shaken at room temperature for 16 h. The resin is filtered off and washed several times with N,N-dimethylformamide, methanol and dichloromethane. Then a solution of 0.65 g (7.00 mmol) of propionic chloride in 5 ml of THF is added, and 1.2 ml (7.00 mmol) of diisopropylethylamine are added. The mixture is shaken at room temperature for 16 h, and then the resin is filtered off and washed several times with N,N-dimethylformamide, methanol and dichloromethane. The product is eliminated from the support resin by treatment with 20 ml of trifluoroacetic acid/dichloromethane (1:1 v/v) at room temperature for 1 h, the polymer is filtered off, and the filtrate is concentrated in vacuo. The product is pure enough for the following reaction.

### Example 15

### (2R)-3-[(4-Chlorophenyl)sulfonyl]-3-(2,5-difluorophenyl)-2-methyl-1-butanol

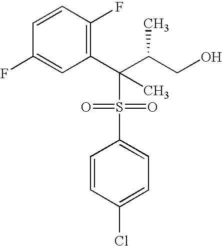

0.45 g (6.65 mmol) of imidazole are added to a solution of 1.2 g (3.33 mmol) of (2R,3S)-3-[(4-chlorophenyl)sulfonyl]-3-(2,5-difluorophenyl)-2-methyl-1-propanol (Example 1-6) in 10 ml of DMF and, after stirring at room temperature for 5 min, 1.00 g (6.65 mmol) of tert-butyldimethylsilyl chloride is added. The mixture is stirred at room temperature for 2 h and then diluted with 50 ml of ethyl acetate and washed three times with saturated sodium bicarbonate solution. The organic phase is dried over sodium sulfate, and the solvent is removed in vacuo. 0.66 g (16.6 mmol) of sodium hydride (60% in mineral oil) is introduced in portions into a solution of the intermediate obtained in this way in 15 ml of THF. The mixture is stirred at room temperature for 30 min and, after addition of 1.05 ml (16.6 mmol) of methyl iodide, stirred at room temperature for a further 16 h. The mixture is subsequently freed of solvent in vacuo. The residue is taken up in 10 ml of a 1 M solution of tetrabutylammonium fluoride in THF. The mixture is stirred at room temperature for 2 h and evaporated in vacuo, and the crude product is purified by preparative HPLC. 985 mg (79% of theory) of the title compound are obtained.

LC/MS (method 3): Rₜ=3.62 min, m/z=375 [M+H]⁺.

### Example 16

### (2R)-3-[(4-Chlorophenyl)sulfonyl]-3-(2,5-difluorophenyl)-2-methylbutyl 3-oxo-1-piperazinecarboxylate

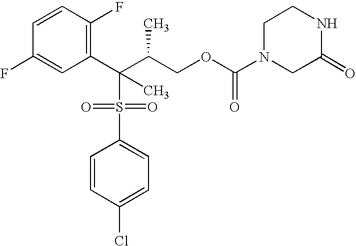

The compound is obtained in analogy to Example 11 and 12 from (2R)-3-[(4-chlorophenyl)sulfonyl]-3-(2,5-difluorophenyl)-2-methyl-1-butanol (Example 15).

¹H-NMR (400 MHz, CDCl₃): δ=7.38-7.23 (m, 4H), 7.09-6.95 (m, 2H), 6.89-6.70 (m, 1H), 5.97-5.88 (br, 1H), 5.03-4.89 (br, 1H), 4.37 (dd, 1H), 4.21 (s, 2H), 3.80-3.72 (m, 2H), 3.54-3.49 (m, 1H), 3.48-3.42 (m, 2H), 3.81 (s, 3H), 0.89 (d, 3H).

LC/MS (method 4): Rₜ=4.12 min, m/z=501 [M+H]⁺.

### Example 17

### (2R,3S)-3-(2,5-Difluorophenyl)-2-methyl-3-{[4-(trifluoromethyl)phenyl]sulfonyl}-propyl 3-oxo-1-piperazinecarboxylate

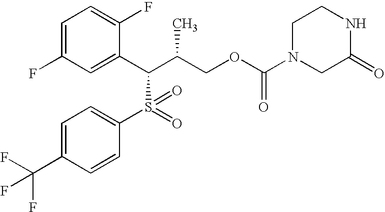

46.0 mg (0.12 mmol) of (2R,3S)-3-(2,5-difluorophenyl)-2-methyl-3-{[4-(trifluoromethyl)phenyl]sulfonyl}-1-propanol (Example 10A-1) are dissolved in 2.0 ml of acetonitrile and, after addition of 0.06 ml (0.35 mmol) of N,N-diisopropylethylamine and 44.8 mg (0.17 mmol) of N,N′-succinimidyl carbonate, stirred at room temperature for 2.5 days. The mixture is diluted with ethyl acetate, washed with saturated sodium bicarbonate solution and saturated sodium chloride solution, dried over magnesium sulfate, filtered and concentrated. 64.1 mg of the intermediate 1-{[(3-(2,5-difluorophenyl)-2-methyl-3-{[4-(trifluoromethyl)phenyl]-sulfonyl}propoxy)carbonyl]oxy}-2,5-pyrrolidinedione are obtained and are reacted further without further purification. 60.0 mg (0.11 mmol) of this intermediate are dissolved in 1.5 ml of acetonitrile and, after addition of 16.8 mg (0.17 mmol) of 2-piperazinone and 0.04 ml (0.20 mmol) of N,N-diisopropylethylamine, stirred at room temperature overnight. The solution is concentrated in vacuo, and the residue is taken up in DMSO and purified by preparative HPLC (RP18 column, eluent acetonitrile/water). 15.7 mg (25.5% of theory) of the title compound are obtained.

¹H-NMR (200 MHz, DMSO-d₆): δ=8.05 (br. s, 1H), 7.90 (d, 2H), 7.80 (d, 2H), 7.45-7.30 (m, 1H), 7.25-7.10 (m, 1H), 7.10-6.90 (m, 1H), 4.90 (d, 1H), 3.95 (dd, 1H), 3.85-3.65 (m, 3H), 3.55-3.40 (m, 2H), 3.20-2.95 (m, 3H), 1.45 (d, 3H).

HPLC (method 1): Rₜ=4.40 min.

MS (ESI pos.): m/z=521 [M+H]⁺.

### Example 18

### 3-[(4-Chlorophenyl)sulfonyl]-3-(2,5-difluorophenyl)propyl 4-hydroxy-1-piperidinecarboxylate

Stage a):

### 2-{1-[(4-Chlorophenyl)sulfonyl]-3-butenyl}-1,4-difluorobenzene

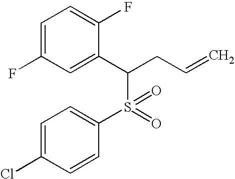

4 g (13.2 mmol) of 2-{[(4-chlorophenyl)sulfonyl]methyl}-1,4-difluorobenzene [prepared in analogy to J. Am. Chem. Soc. 66, 1132-1136 (1944) from sodium 4-chlorophenylsulfinate and 2,5-difluorobenzyl chloride] are dissolved in 100 ml of dry tetrahydrofuran and cooled to −78° C., and 8.67 ml of n-butyllithium (1.6 M solution in hexane; 13.9 mmol) are added. The mixture is warmed to room temperature, stirred for 15 min, again cooled to −78° C. and, after addition of 1.2 ml (13.9 mmol) of allyl bromide, warmed again to room temperature. After 12 h at room temperature, water and dichloromethane are added, and the organic phase is separated off, washed with saturated sodium chloride solution, dried over magnesium sulfate and concentrated. Purification of the residue by chromatography (silica gel, mobile phase: cyclohexane/ethyl acetate 50:1→10:1) affords 4.58 g (99.6% of theory) of the title compound.

LC/MS (method 3): Rₜ=4.14 min, m/z=343 [M+H]⁺.

Stage b):

### 3-[(4-Chlorophenyl)sulfonyl]-3-(2,5-difluorophenyl)-1-propanol

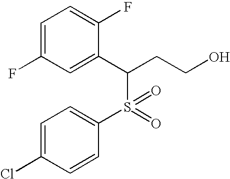

2.15 g (6.28 mmol) of 2-{1-[(4-chlorophenyl)sulfonyl]-3-butenyl}-1,4-difluorobenzene are dissolved in 25 ml of tetrahydrofuran and, after addition of 4.03 g (18.8 mmol) of sodium periodate and 0.6 ml of osmium tetroxide (2.5% strength solution in 2-methyl-2-propanol; 0.06 mmol), are stirred at room temperature for 5 h. Addition of 25 ml of water is followed by extraction with dichloromethane, and the organic phase is washed with saturated sodium bicarbonate solution, dried over magnesium sulfate and concentrated. The residue is dissolved in 30 ml of tetrahydrofuran/water (2:1) and, after addition of 237 mg (6.28 mmol) of sodium borohydride, stirred at room temperature overnight. The mixture is diluted with water and dichloromethane, and the organic phase is washed with saturated sodium bicarbonate solution, dried over magnesium sulfate and concentrated.

Purification of the residue by chromatography (silica gel, mobile phase: cyclohexane/ethyl acetate 25:1→10:1) affords 1.22 g (56% of theory) of the title compound.

HPLC (method 1): Rₜ=4.35 min.

MS (ESI pos.): m/z=347 [M+H]⁺.

Stage c):

### 3-[(4-Chlorophenyl)sulfonyl]-3-(2,5-difluorophenyl)propyl 4-hydroxy-1-piperidinecarboxylate

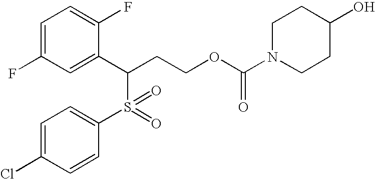

100 mg (0.29 mmol) of 3-[(4-chlorophenyl)sulfonyl]-3-(2,5-difluorophenyl)-1-propanol, 70 μl (0.87 mmol) of pyridine and 0.5 ml of acetonitrile in 2 ml of tetrahydrofuran are cooled to 0° C. and, after addition of 116 mg (0.58 mmol) of 4-nitrophenyl chloroformate, stirred at 55° C. for 6 h. After cooling to room temperature, 175 mg (1.73 mmol) of 4-hydroxypiperidine in 1 ml of tetrahydrofuran are added and stirred overnight. The reaction mixture is, concentrated, taken up in dichloromethane, washed with saturated sodium bicarbonate solution, dried over magnesium sulfate and concentrated. Purification of the residue by preparative HPLC (RP18 column, eluent acetonitrile/water) affords 72.8 mg (51% of theory) of the title compound.

¹H-NMR (300 MHz, DMSO-d₆): δ=7.7-7.6 (m, 4H), 7.4-7.1 (m, 3H), 4.85 (t, 1H), 4.1-4.0 (m, 1H), 3.9-3.8 (m, 1H), 3.6-3.2 (m, 5H), 2.85 (br. s, 2H), 2.55-2.45 (m, 1H), 1.65-1.55 (m, 2H), 1.25-1.1 (m, 2H).

LC/MS (method 4): Rₜ=3.59 min, m/z=474 [M+H]⁺.

### Example 19

### 3-[(4-Chlorophenyl)sulfonyl]-3-(2,5-difluorophenyl)butyl 1-pyrrolidinecarboxylate

Stage a):

### 2-{1-[(4-Chlorophenyl)sulfonyl]-1-methyl-3-butenyl}-1,4-difluorobenzene

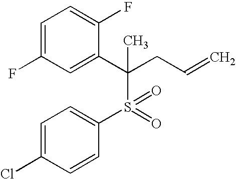

6.1 g (17.8 mmol) of 2-{1-[(4-chlorophenyl)sulfonyl]-3-butenyl}-1,4-difluorobenzene (Example 18/stage a) are dissolved in 122 ml of tetrahydrofuran and cooled to 0° C. and, after addition of 1.07 g of sodium hydride (60% in mineral oil; 26.7 mmol) and 1.33 ml (21.4 mmol) of methyl iodide, stirred at room temperature overnight. Addition of methanol and water is followed by extraction with ethyl acetate, drying of the organic phase over magnesium sulfate and concentration. 5.84 g (88% of theory) of the title compound are obtained.

LC/MS (method 4): Rₜ=4.50 min, m/z=487 [M+Na]⁺.

Stage b):

### 3-[(4-Chlorophenyl)sulfonyl]-3-(2,5-difluorophenyl)-1-butanol

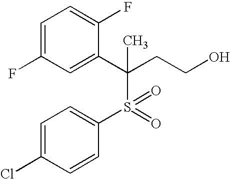

2.02 g (5.66 mmol) of 2-{1-[(4-Chlorophenyl)sulfonyl]-1-methyl-3-butenyl}-1,4-difluorobenzene are dissolved in 21 ml of tetrahydrofuran and, after addition of 3.63 g (17.0 mmol) of sodium periodate and 0.55 ml of osmium tetroxide (2.5% strength solution in 2-methyl-2-propanol; 0.06 mmol), stirred at room temperature overnight. Addition of water is followed by extraction with dichloromethane, and the organic phase is washed with saturated sodium bicarbonate solution, dried over magnesium sulfate and concentrated. The residue is dissolved in 21 ml of tetrahydrofuran/water (2:1) and, after addition of 213 mg (5.66 mmol) of sodium borohydride, stirred at room temperature overnight. The mixture is diluted with water and dichloromethane, and the organic phase is washed with saturated sodium bicarbonate solution, dried over magnesium sulfate and concentrated. Purification of the residue by chromatography (silica gel, mobile phase: cyclohexane/ethyl acetate 25:1→10:1) affords 1.22 g (56% of theory) of the title compound.

LC/MS (method 3): Rₜ=3.38 min, m/z=361 [M+H]⁺

Stage c):

### 3-[(4-Chlorophenyl)sulfonyl]-3-(2,5-difluorophenyl)butyl 1-pyrrolidinecarboxylate

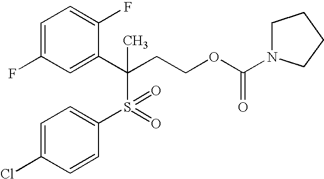

50 mg (0.14 mmol) of 3-[(4-chlorophenyl)sulfonyl]-3-(2,5-difluorophenyl)-1-butanol are dissolved in 5 ml of dry tetrahydrofuran and, after addition of 11 mg of sodium hydride (60% in mineral oil; 0.28 mmol) and, after 30 min, 37 mg (0.28 mmol) of 1-pyrrolidinecarbonyl chloride, stirred at room temperature overnight. Addition of methanol and water is followed by extraction with ethyl acetate, drying of the organic phase over magnesium sulfate and concentration. Purification by preparative HPLC (RP18 column, eluent acetonitrile/water) affords 28 mg (44% of theory) of the title compound.

HPLC (method 1): Rₜ=4.87 min.

MS (DCI): m/z=475 [M+NH₄]⁺.

### Example 20

### 3-[(4-Chlorophenyl)sulfonyl]-3-(2,5-difluorophenyl)-2,2-dimethylpropyl 1-pyrrolidinecarboxylate

Stage a):

### Methyl 3-(2,5-difluorophenyl)-3-hydroxy-2,2-dimethylpropionate

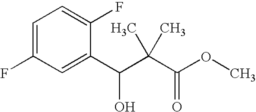

A solution of 2.00 g (14.07 mmol) of 2,5-difluorobenzaldehyde in 100 ml of absolute dichloromethane is cooled to −78° C., and 1.54 ml (14.07 mmol) of titanium(IV) chloride are added. 2.57 ml (12.67 mmol) of 1-methoxy-2-methyl-1-trimethylsiloxypropene in 50 ml of absolute dichloromethane are added dropwise. After one hour at −78° C., 100 ml of water are used for quenching, and the mixture is slowly warmed to room temperature. The phases are separated and the aqueous phase is extracted with dichloromethane. The combined organic phases are dried over magnesium sulfate and concentrated. Purification of the residue by chromatography (silica gel, mobile phase: cyclohexane/ethyl acetate 20:1, 10:1) affords 2.83 g (82% of theory) of the title compound.

HPLC (method 1): Rₜ=4.37 min.

MS (DCI): m/z=245 [M+NH₄]⁺.

Stage b):

### Methyl 3-[(4-chlorophenyl)sulfanyl]-3-(2,5-difluorophenyl)-2,2-dimethylpropionate

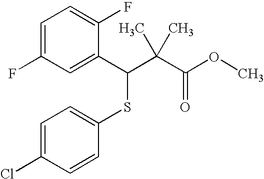

0.70 g (2.87 mmol) of methyl 3-(2,5-difluorophenyl)-3-hydroxy-2,2-dimethylpropionate and 7.52 g (28.7 mmol) of triphenylphosphine are dissolved in 40 ml of absolute tetrahydrofuran and cooled to 0° C. 5.54 ml (28.7 mmol) of diisopropyl azodicarboxylate and, after 10 minutes, 0.83 g (5.73 mmol) of 4-chlorothiophenol are added. The mixture is warmed to room temperature and stirred at this temperature overnight. After addition of water, the aqueous phase is extracted with dichloromethane, and the combined organic phases are dried over magnesium sulfate and concentrated. 0.80 g (75% of theory) of the title compound is obtained.

HPLC (method 1): Rₜ=5.7 min.

MS (DCI): m/z=388 [M+NH₄]⁺.

Stage c):

### 3-[(4-Chlorophenyl)sulfanyl]-3-(2,5-difluorophenyl)-2,2-dimethyl-1-propanol

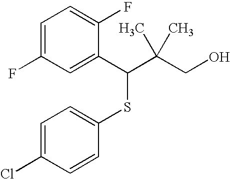

Under an argon atmosphere 0.86 ml (0.86 mmol) of a 1 M solution of lithium aluminum hydride in tetrahydrofuran is diluted with 5 ml of absolute diethyl ether and heated to reflux. A solution of 0.40 g (1.08 mmol) of methyl 3-[(4-chlorophenyl)sulfanyl]-3-(2,5-difluorophenyl)-2,2-dimethylpropionate in 5 ml of absolute diethyl ether is slowly added dropwise. The mixture is heated to reflux overnight and, after cooling to room temperature, quenched with water. Addition of 0.1 M hydrochloric acid is followed by extraction with ethyl acetate, drying over magnesium sulfate and concentration. Purification of the residue by preparative HPLC (RP18 column, eluent acetonitrile/water) affords 0.23 g (94% of theory) of the title compound.

HPLC (method 1): Rₜ=5.25 min.

MS (DCI): m/z=360 [M+NH₄]⁺.

Stage d):

### 3-[(4-Chlorophenyl)sulfonyl]-3-(2,5-difluorophenyl)-2,2-dimethyl-1-propanol

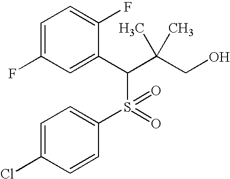

0.20 g (0.59 mmol) of 3-[(4-chlorophenyl)sulfanyl]-3-(2,5-difluorophenyl)-2,2-dimethyl-1-propanol is dissolved in 10 ml of dichloromethane and cooled to 0° C. 0.32 g (1.29 mmol) of meta-chloroperbenzoic acid is added, and the mixture is stirred at room temperature overnight. Addition of saturated sodium thiosulfate solution is followed by extraction with dichloromethane. The combined organic phases are washed with saturated sodium bicarbonate solution, dried over magnesium sulfate and concentrated. Chromatographic purification of the residue by preparative HPLC (RP18 column, eluent acetonitrile/water) affords 0.16 g (98% of theory) of the title compound.

LC/MS (method 2): Rₜ=3.87 min, m/z=397 [M+Na]⁺.

Stage e):

### 3-[(4-Chlorophenyl)sulfonyl]-3-(2,5-difluorophenyl)-2,2-dimethylpropyl 1-pyrrolidinecarboxylate

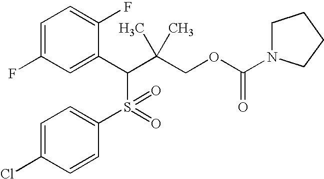

70 mg (0.19 mmol) of 3-[(4-chlorophenyl)sulfonyl]-3-(2,5-difluorophenyl)-2,2-dimethyl-1-propanol are dissolved in 3.0 ml of absolute THF and cooled to 0° C. 11.2 mg of sodium hydride (60% in mineral oil; 0.28 mmol) and 45 μl (0.37 mmol) of pyrrolidinecarbonyl chloride are added. The mixture is stirred at room temperature for 5 h and, after addition of methanol and water, extracted with ethyl acetate. The organic phases are dried over magnesium sulfate and concentrated. Chromatographic purification of the residue by preparative HPLC (RP18 column, eluent acetonitrile/water) affords 63.4 mg (98% of theory) of the title compound.

HPLC (method 1): Rₜ=5.12 min.

MS (DCI): m/z=489 [M+NH₄]⁺

¹H-NMR (200 MHz, DMSO-d₆): δ=7.68-7.50 (m, 5H), 7.32-7.02 (m, 2H), 4.93 (s, 1H), 4.19 (d, 1H, ³J=16.0 Hz), 3.83 (d, 1H, ³J=16.0 Hz), 3.30-3.20 (m, 4H), 1.92-1.73 (m, 4H), 1.46 (s, 3H), 1.03 (s, 3H).

### Example 21

### (2R,3S)-3-[(4-Chlorophenyl)sulfonyl]-3-(2,5-difluorophenyl)-2-methylpropyl 4-cyanophenylcarbamate

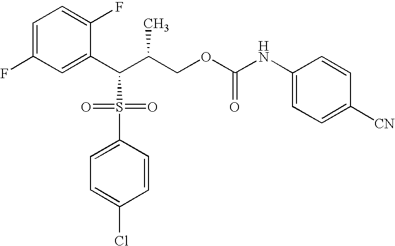

2 mg (0.02 mmol) of N,N-dimethylaminopyridine and 29 mg (0.20 mmol) of p-cyanophenyl isocyanate are added to a solution of 60 mg (0.17 mmol) of (2R,3S)-3-[(4-chlorophenyl)sulfonyl]-3-(2,5-difluorophenyl)-2-methyl-1-propanol (Example 1-6) in 2 ml of THF. The mixture is stirred at room temperature for 4 h and then evaporated to dryness in vacuo. The residue is taken up in acetonitrile, and the crude product is then purified by preparative HPLC. 77 mg (91% of theory) of a colorless solid are obtained.

¹H-NMR (200 MHz, DMSO-d₆): δ=7.72 (d, 1H), 7.62-7.49 (m, 3H), 7.47-7.31 (m, 1H), 7.27-7.10 (m, 1H), 7.08-6.91 (m, 1H), 4.78 (d, 1H), 4.00 (dd, 1H), 3.80 (dd, 1H), 3.13-2.95 (m, 1H), 1.49 (d, 1H).

LC/MS (method 7): Rₜ=4.89 min, m/z=504 [M+H]⁺.

The compounds listed in the following table are obtained in analogy to the examples described above; the synthetic building blocks required to prepare the final compounds are either commercially available, described in the literature or can be prepared in analogy to processes known from the literature.

| | | | | | | LC/MS or |
|---|---|---|---|---|---|---|
| Exam- | | | | LC/MS or | Rₜ LC/MS | MS (ESI |
| ple | Synthesis | | | HPLC | or HPLC | pos.) |
| No. | method | Structure | Isomer | method | [min] | [M + H]⁺ |
| 22 | Analogous to Example 1 | 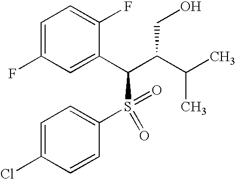 | Diastereomer 1, racemic | 1 | 4.72 | 389 |
| 23 | Analogous to Example 1 | 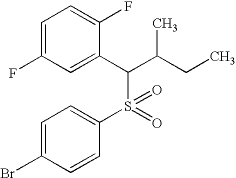 | Diastereomer 1, racemic | 1 | 4.56 | 405 |
| 24 | Analogous to Example 1 | 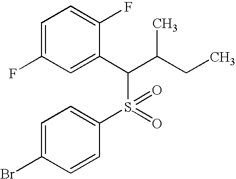 | Diastereomer 2, racemic | 1 | 4.61 | 405 |
| 25 | Analogous to Example 1 | 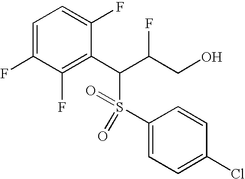 | Diastereomer mixture, racemic | 1 | 4.54 | 379 |
| 26 | Analogous to Example 1 | 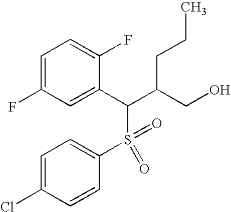 | Diastereomer mixture, racemic | 1 | 4.89 | 389 |
| 27 | Analogous to Example 1 | 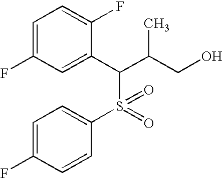 | Diastereomer mixture, racemic | 3 | 3.38 | 345 |
| 28 | Analogous to Example 16 | 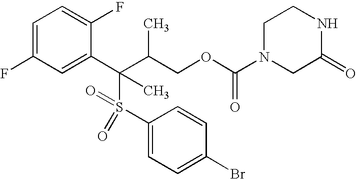 | Diastereomer 2, racemic | 1 | 4.43 | 545 |
| 29 | Analogous to Example 16 | 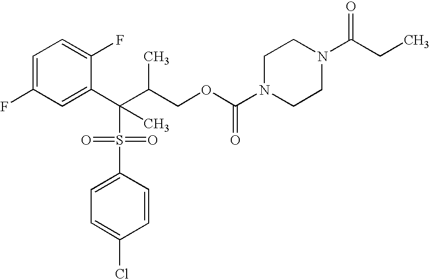 | Diastereomer 2, racemic | 4 | 4.31 | 543 |
| 30 | Analogous to Example 16 | 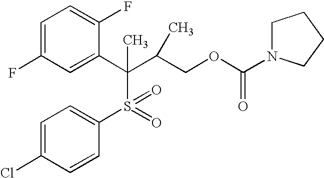 | Diastereomer mixture, racemic | 3 | 4.12 and 4.22 | 472 |
| 31 | Analogous to Example 16 | 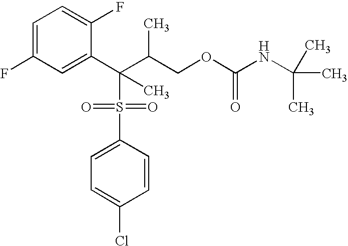 | Diastereomer 2, racemic | 4 | 4.63 | 496 [M +Na]⁺ |
| 32 | Analogous to Example 16 | 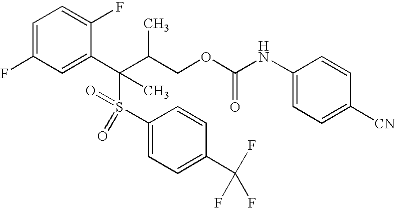 | Diastereomer 1, racemic | 2 | 4.43 | 575 [M +Na]⁺ |
| 33 | Analogous to Example 16 | 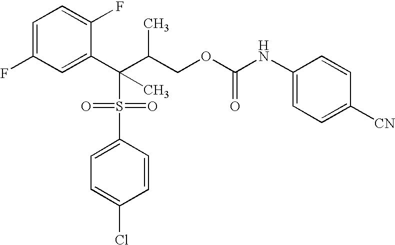 | Diastereomer 2, racemic | 3 | 4.28 | 519 |
| 34 | Analogous to Example 16 | 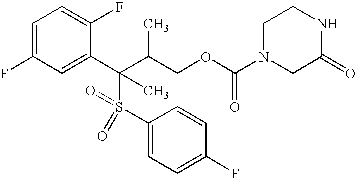 | Diastereomer 2, racemic | 1 | 4.16 | 485 |
| 35 | Analogous to Example 16 | 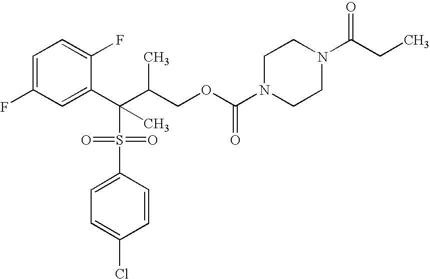 | Diastereomer 1, racemic | 4 | 4.25 | 543 |
| 36 | Analogous to Example 16 | 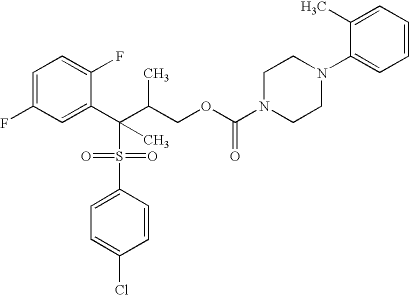 | Diastereomer 2, racemic | 8 | 4.75 | 577 |
| 37 | Analogous to Example 16 | 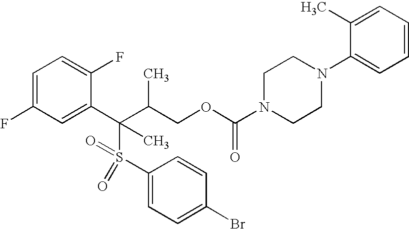 | Diastereomer 2, racemic | 1 | 5.45 | 621 |
| 38 | Analogous to Example 16 | 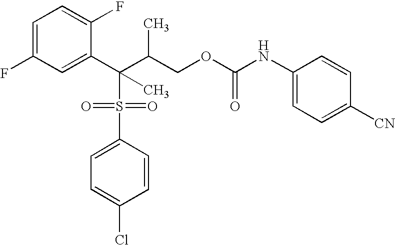 | Diastereomer 2, racemic | 3 | 4.20 | 519 |
| 39 | Analogous to Example 16 | 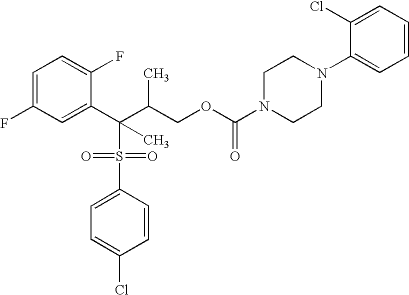 | Diastereomer 2, racemic | 4 | 4.84 | 597 |
| 40 | Analogous to Example 16 | 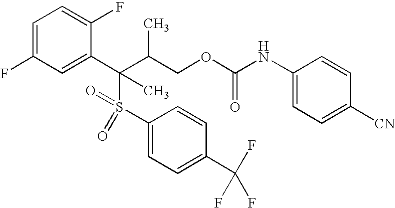 | Diastereomer 2, racemic | 1 | 5.19 | 575 [M +Na]⁺ |
| 41 | Analogous to Example 5A | 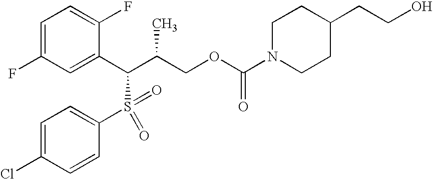 | Diastereomer 1, racemic | 1 | 4.67 | 516 |
| 42 | Analogous to Example 5A | 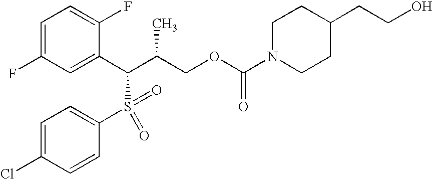 | Diastereomer 1, Enantiomer A | 1 | 4.68 | 516 |
| 43 | Analogous to Example 5A | 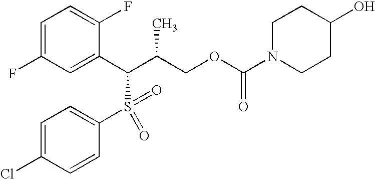 | Diastereomer 1, Enantiomer A | 1 | 4.39 | 488 |
| 44 | Analogous to Example 5A | 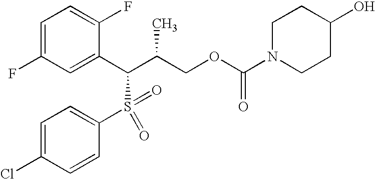 | Diastereomer 1, racemic | 1 | 4.42 | 488 |
| 45 | Analogous to Example 5A | 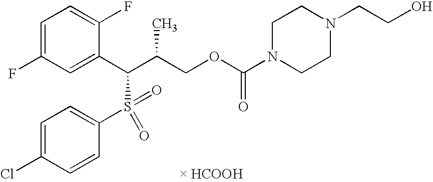 | Diastereomer 1, racemic | 1 | 4.24 | 517 |
| 46 | Analogous to Example 5A | 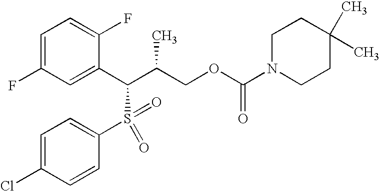 | Diastereomer 1, racemic | 1 | 5.45 | 500 |
| 47 | Analogous to Example 5A | 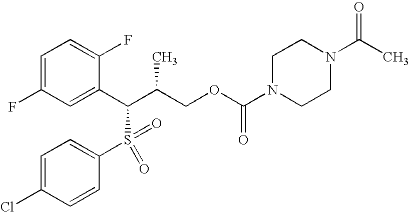 | Diastereomer 1, Enantiomer A | 1 | 4.48 | 515 |
| 48 | Analogous to Example 5A | 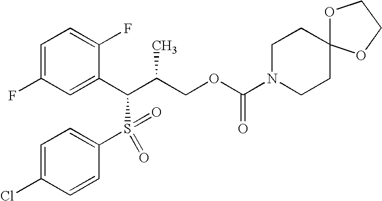 | Diastereomer 1, Enantiomer A | 1 | 4.90 | 530 |
| 49 | Analogous to Example 5A | 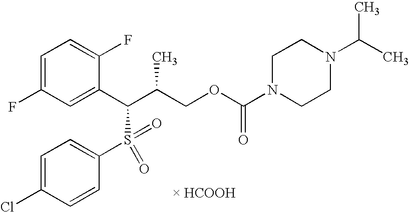 | Diastereomer 1, racemic | 1 | 4.42 | 515 |
| 50 | Analogous to Example 5A | 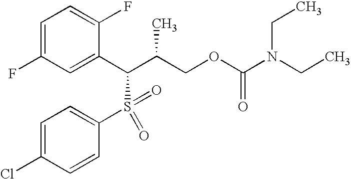 | Diastereomer 1, Enantiomer A | 1 | 5.14 | 460 |
| 51 | Analogous to Example 5A | 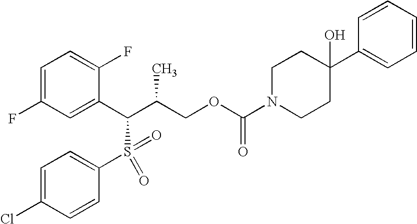 | Diastereomer 1, Enantiomer A | 1 | 4.79 | 564 |
| 52 | Analogous to Example 5A | 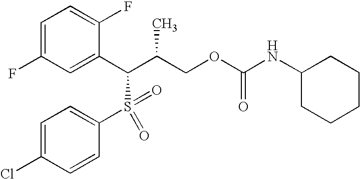 | Diastereomer 1, racemic | 1 | 5.32 | 486 |
| 53 | Analogous to Example 5A | 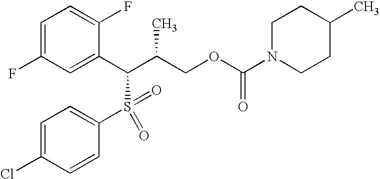 | Diastereomer 1, racemic | 1 | 5.28 | 486 |
| 54 | Analogous to Example 5A | 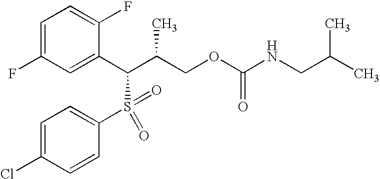 | Diastereomer 1, Enantiomer A | 1 | 4.84 | 460 |
| 55 | Analogous to Example 5A | 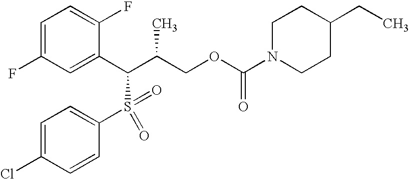 | Diastereomer 1, racemic | 1 | 5.45 | 500 |
| 56 | Analogous to Example 5A | 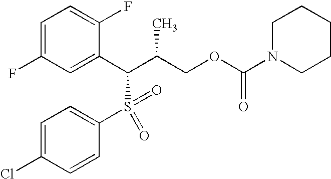 | Diastereomer 1, racemic | 1 | 5.11 | 472 |
| 57 | Analogous to Example 5A | 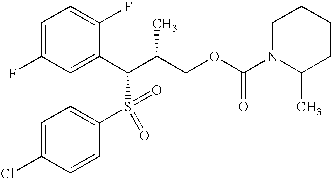 | Diastereomer 1, racemic | 1 | 5.24 | 486 |
| 58 | Analogous to Example 5A | 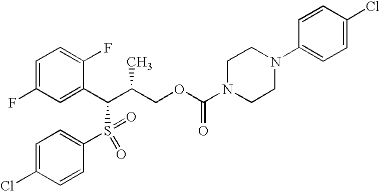 | Diastereomer 1, Enantiomer A | 1 | 5.41 | 583 |
| 59 | Analogous to Example 5A | 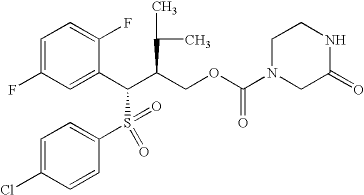 | Diastereomer 1, racemic | 1 | 4.50 | 515 |
| 60 | Analogous to Example 5A | 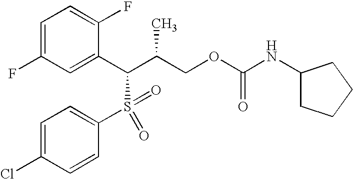 | Diastereomer 1, Enantiomer A | 1 | 4.95 | 472 |
| 61 | Analogous to Example 5A | 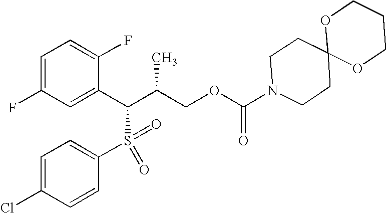 | Diastereomer 1, Enantiomer A | 1 | 4.76 | 544 |
| 62 | Analogous to Example 5A | 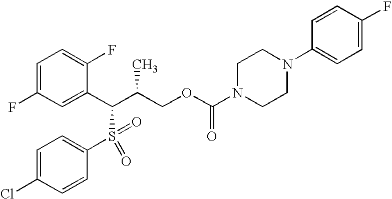 | Diastereomer 1, Enatiomer A | 1 | 4.96 | 567 |
| 63 | Analogous to Example 5A | 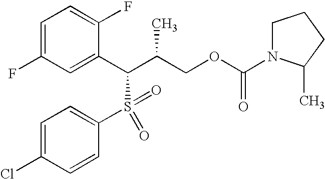 | Diastereomer 1, racemic | 1 | 5.06 | 472 |
| 64 | Analogous to Example 5A | 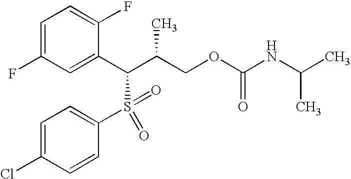 | Diastereomer 1, Enantiomer A | 1 | 4.91 | 446 |
| 65 | Analogous to Example 5A | 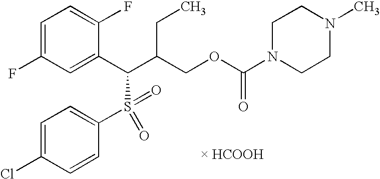 | Diastereomer mixture, racemic | 1 | 4.36 | 501 |
| 66 | Analogous to Example 5A | 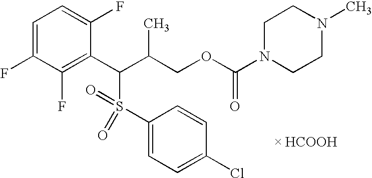 | Diastereomer mixture, racemic | 1 | 4.32 | 505 |
| 67 | Analogous to Example 5A | 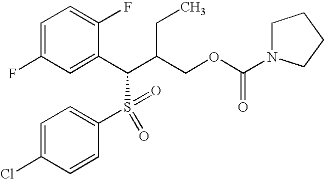 | Diastereomer mixture, racemic | 1 | 5.08 | 472 |
| 68 | Analogous to Example 5A | 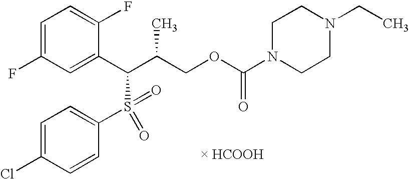 | Diastereomer 1, racemic | 1 | 4.31 | 501 |
| 69 | Analogous to Example 5A | 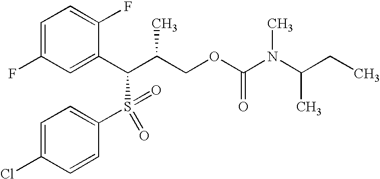 | Diastereomer 1, racemic | 1 | 5.17 | 474 |
| 70 | Analogous to Example 5A | 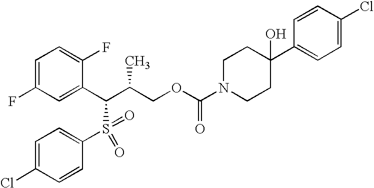 | Diastereomer 1, Enantiomer A | 1 | 5.04 | 598 |
| 71 | Analogous to Example 5A | 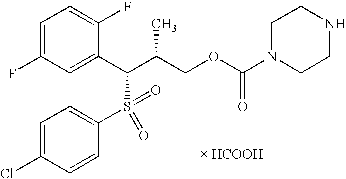 | Diastereomer 1, racemic | 1 | 4.31 | 473 |
| 72 | Analogous to Example 5A | 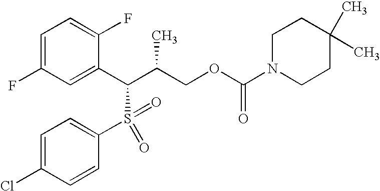 | Diastereomer 1, Enantiomer A | 1 | 5.48 | 500 |
| 73 | Analogous to Example 5A | 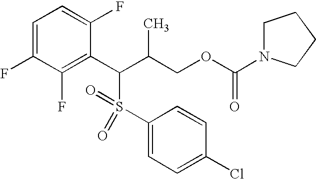 | Diastereomer mixture, racemic | 1 | 4.97 | 476 |
| 74 | Analogous to Example 5A | 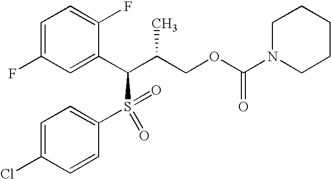 | Diastereomer 2, racemic | 1 | 5.06 | 472 |
| 75 | Analogous to Example 5A | 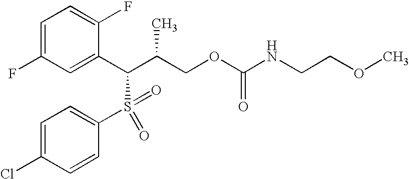 | Diastereomer 1, Enantiomer A | 1 | 4.53 | 462 |
| 76 | Analogous to Example 10-1 | 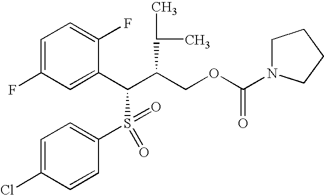 | Diastereomer 1, racemic | 1 | 5.35 | 486 |
| 77 | Analogous to Example 10-2 | 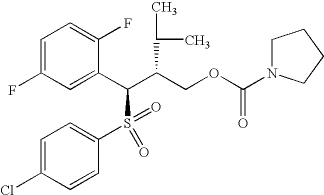 | Diastereomer 2, racemic | 1 | 5.33 | 486 |
| 78 | Analogous to Example 5A | 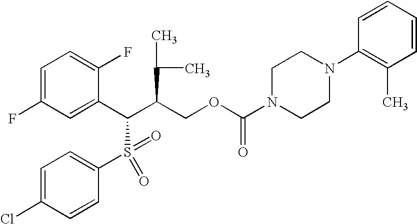 | Diastereomer 2, racemic | 1 | 5.54 | 591 |
| 79 | Analogous to Example 5A | 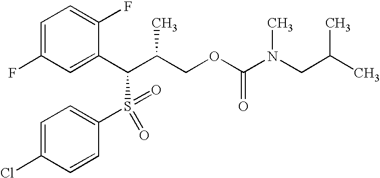 | Diastereomer 1, racemic | 1 | 5.35 | 474 |
| 80 | Analogous to Example 5A | 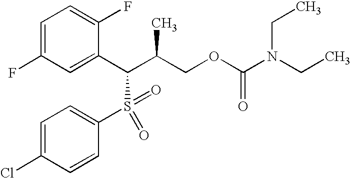 | Diastereomer 2, Enantiomer B | 1 | 5.14 | 460 |
| 81 | Analogous to Example 5A | 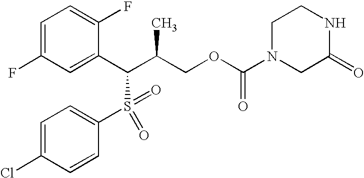 | Diastereomer 2, Enantiomer B | 1 | 4.31 | 487 |
| 82 | Analogous to Example 5A | 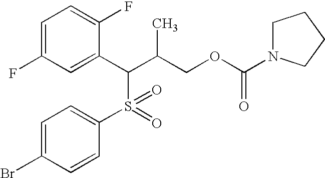 | Diastereomer mixture, racemic | 1 | 4.98 | 502 |
| 83 | Analogous to Example 5A | 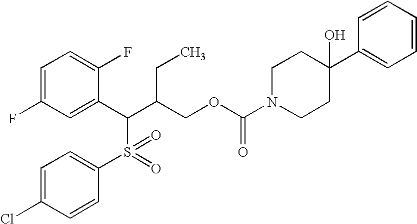 | Diastereomer mixture, racemic | 3 | 4.3 | 578 |
| 84 | Analogous to Example 5A | 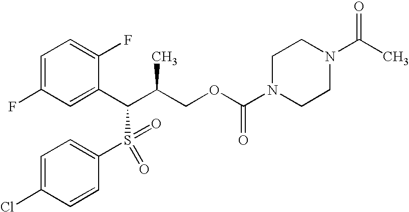 | Diastereomer 2, racemic | 1 | 4.48 | 515 |
| 85 | Analogous to Example 5A | 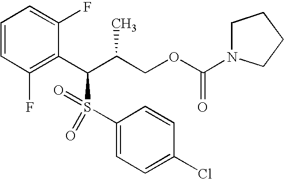 | Diastereomer 2, racemic | 1 | 4.68 | 458 |
| 86 | Analogous to Example 5A | 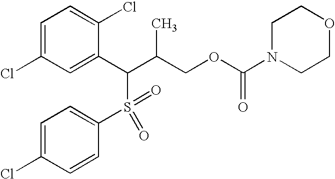 | Diastereomer mixture, racemic | 1 | 5.04 and 5.09 | 506 |
| 87 | Analogous to Example 5A | 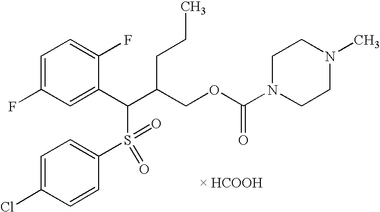 | Diastereomer mixture, racemic | 1 | 4.47 | 515 |
| 88 | Analogous to Example 5A | 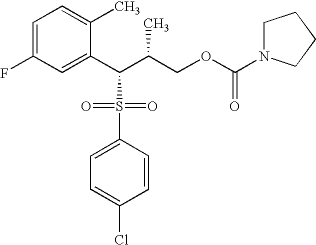 | Diastereomer 1, Enantiomer A | 4 | 4.72 | 454 |
| 89 | Analogous to Example 5A | 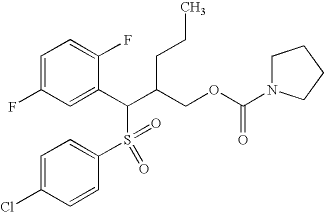 | Diastereomer mixture, racemic | 1 | 5.30 | 486 |
| 90 | Analogous to Example 5A | 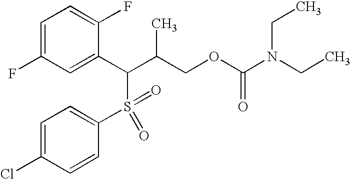 | Diastereomer 2, racemic | 1 | 5.14 | 460 |
| 91 | Analogous to Example 5A | 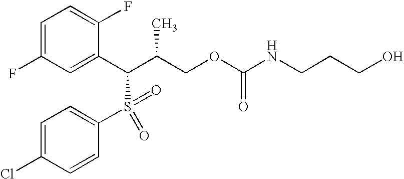 | Diastereomer 1, Enantiomer A | 1 | 4.25 | 462 |
| 92 | Analogous to Example 5A | 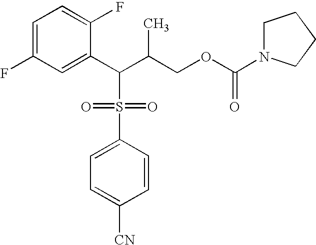 | Diastereomer mixture, racemic | 6 | 3.83 | 449 |
| 93 | Analogous to Example 5A | 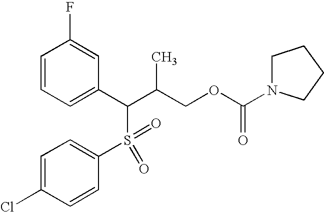 | Diasteromer mixture, racemic | 1 | 4.98 and 5.04 | 440 |
| 94 | Analogous to Example 5A | 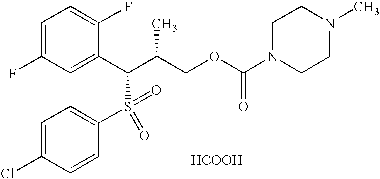 | Diastereomer 1, racemic | 1 | 4.26 | 487 |
| 95 | Analogous to Example 5A | 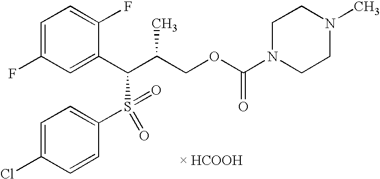 | Diastereomer 1, Enantiomer A | 1 | 4.30 | 487 |
| 96 | Analogous to Example 5A | 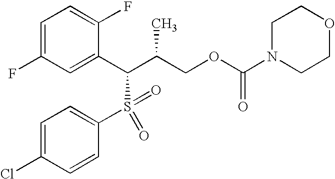 | Diastereomer 1, racemic | 1 | 4.68 | 474 |
| 97 | Analogous to Example 5A | 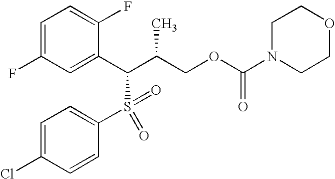 | Diastereomer 1, Enantiomer A | 1 | 4.73 | 474 |
| 98 | Analogous to Example 11 | 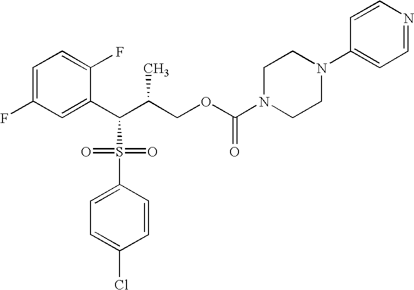 | Diastereomer 1, racemic | 3 | 2.85 | 550 |
| 99 | Analogous to Example 11 | 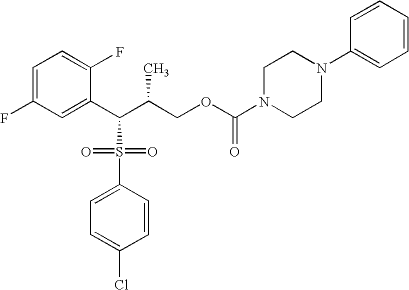 | Diastereomer 1, racemic | 6 | 4.21 | 549 |
| 100 | Analogous to Example 11 | 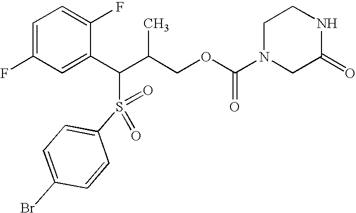 | Diastereomer 1, racemic | 1 | 4.31 | 531 |
| 101 | Analogous to Example 11 | 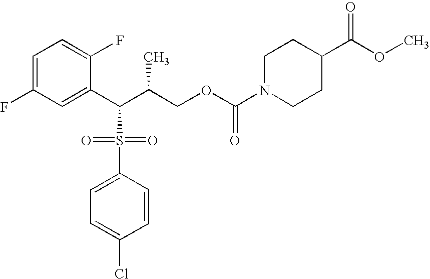 | Diastereomer 1, Enantiomer A | 5 | 4.37 | 530 |
| 102 | Analogous to Example 11 | 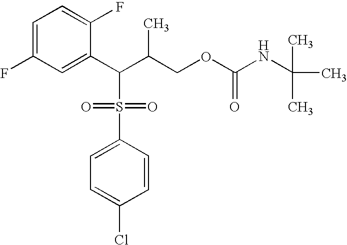 | Diastereomer mixture, racemic | 3 | 4.28 | 460 |
| 103 | Analogous to Example 11 | 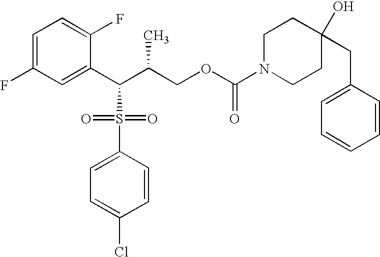 | Diastereomer 1, Enantiomer A | 3 | 4.24 | 578 |
| 104 | Analogous to Example 11 | 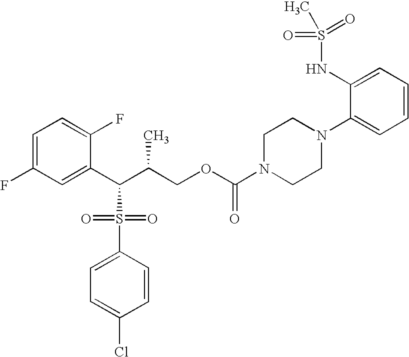 | Diastereomer 1, Enantiomer A | 9 | 3.79 | 642 |
| 105 | Analogous to Example 11 | 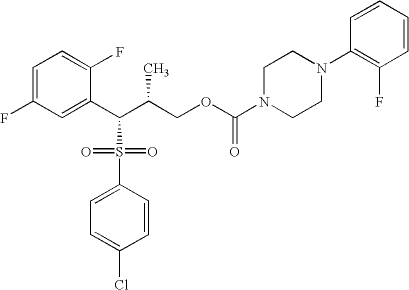 | Diastereomer 1, racemic | 6 | 4.26 | 567 |
| 106 | Analogous to Example 11 | 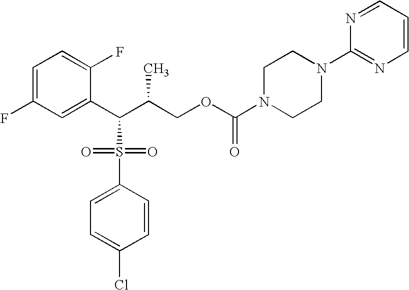 | Diastereomer 1, racemic | 3 | 3.97 | 551 |
| 107 | Analogous to Example 11 | 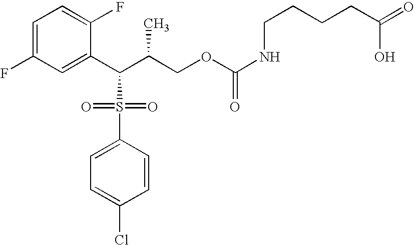 | Diastereomer 1, Enantiomer A | 2 | 3.55 | 504 |
| 108 | Analogous to Example 11 | 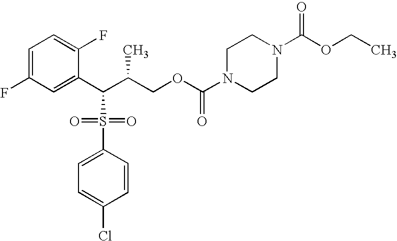 | Diastereomer 1, Enantiomer A | 3 | 4.07 | 545 |
| 109 | Analogous to Example 11 | 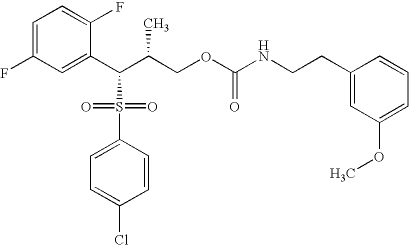 | Diastereomer 1, Enantiomer A | 8 | 5.16 | 560 [M +Na]⁺ |
| 110 | Analogous to Example 11 | 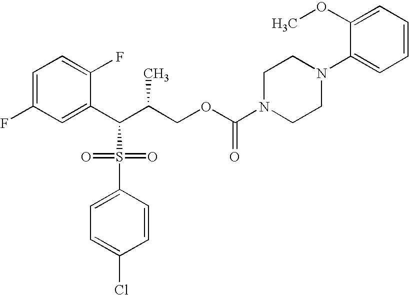 | Diastereomer 1, Enantiomer A | 9 | 3.97 | 579 |
| 111 | Analogous to Example 11 | 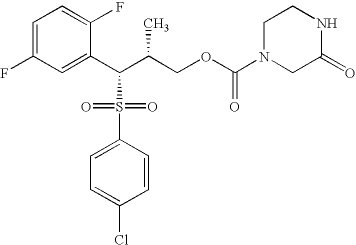 | Diastereomer 1, racemic | 8 | 4.10 | 487 |
| 112 | Analogous to Example 11 | 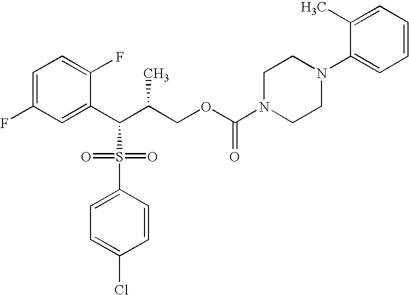 | Diastereomer 1, Enantiomer A | 8 | 5.80 | 563 |
| 113 | Analogous to Example 11 | 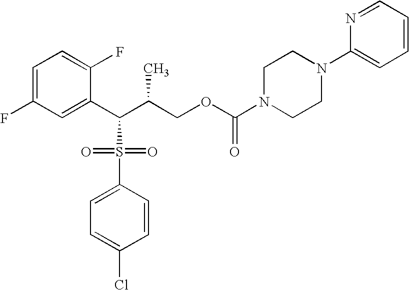 | Diastereomer 1, Enantiomer A | 8 | 3.39 | 550 |
| 114 | Analogous to Example 11 | 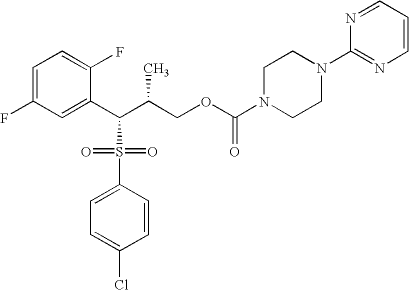 | Diastereomer 1, Enantiomer A | 6 | 4.01 | 551 |
| 115 | Analogous to Example 11 | 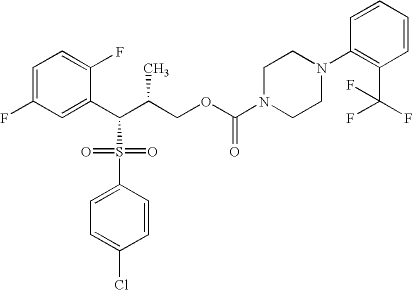 | Diastereomer 1, racemic | 6 | 4.44 | 617 |
| 116 | Analogous to Example 11 | 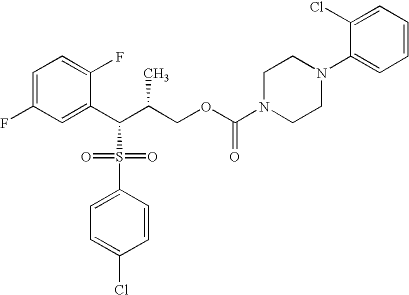 | Diastereomer 1, Enantiomer A | 9 | 4.36 | 583 |
| 117 | Analogous to Example 11 | 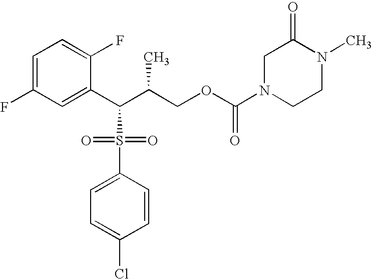 | Diastereomer 1, Enantiomer A | 4 | 3.63 | 501 |
| 118 | Analogous to Example 11 | 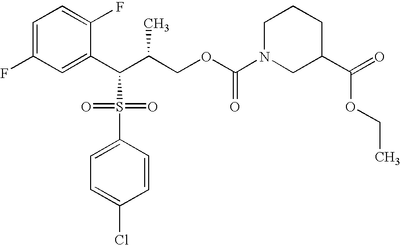 | Diastereomer mixture, Enantiomer A | 3 | 4.43 | 544 |
| 119 | Analogous to Example 11 | 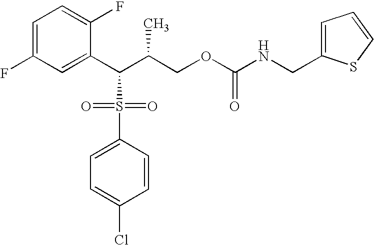 | Diastereomer 1, Enantiomer A | 8 | 5.04 | 522 [M +Na]⁺ |
| 120 | Analogous to Example 11 | 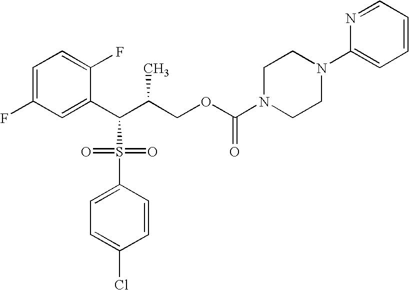 | Diastereomer 1, racemic | 8 | 3.91 | 550 |
| 121 | Analogous to Example 11 | 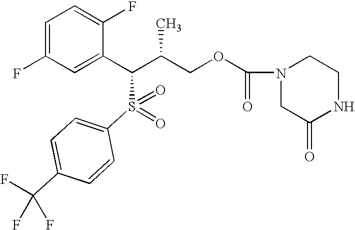 | Diastereomer 1, racemic | 1 | 4.4 | 521 |
| 122 | Analogous to Example 16 | 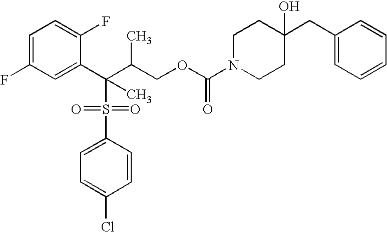 | Diastereomer 2, racemic | 4 | 4.65 | 614 [M +Na]⁺ |
| 123 | Analogous to Example 11 | 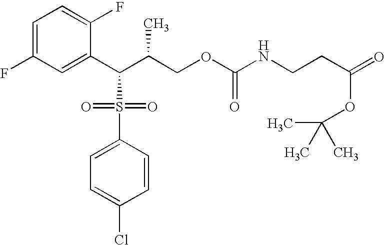 | Diastereomer 1, Enantiomer A | 5 | 4.44 | 532 |
| 124 | Analogous to Example 11 | 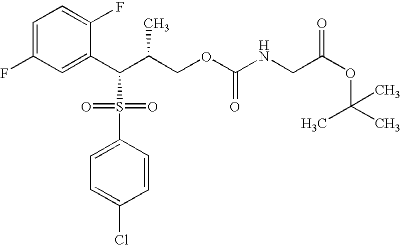 | Diastereomer 1, Enantiomer A | 5 | 4.40 | 518 |
| 125 | Analogous to Example 11 | 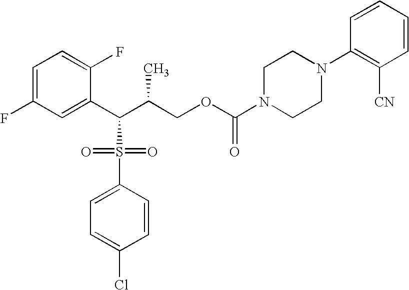 | Diastereomer 1, racemic | 3 | 4.42 | 574 |
| 126 | Analogous to Example 11 | 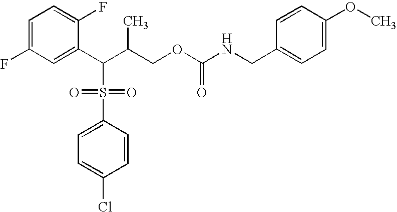 | Diastereomer mixture, racemic | 8 | 5.10 | 524 |
| 127 | Analogous to Example 11 | 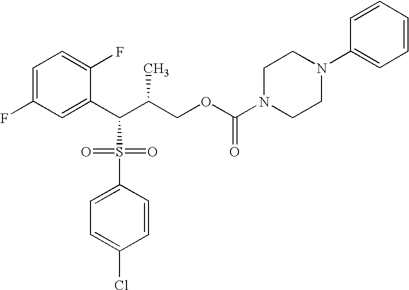 | Diastereomer 1, Enantiomer A | 3 | 4.52 | 549 |
| 128 | Analogous to Example 11 | 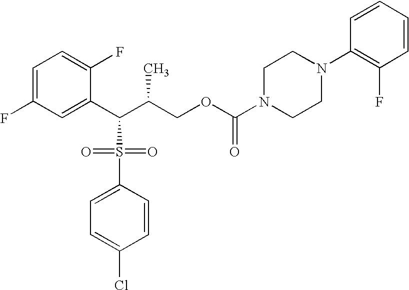 | Diastereomer 1, Enantiomer A | 3 | 4.59 | 567 |
| 129 | Analogous to Example 11 | 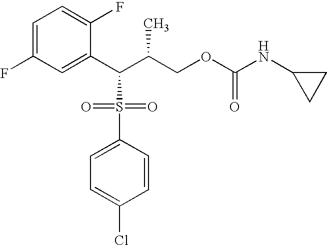 | Diastereomer 1, racemic | 3 | 3.88 | 444 |
| 130 | Analogous to Example 11 | 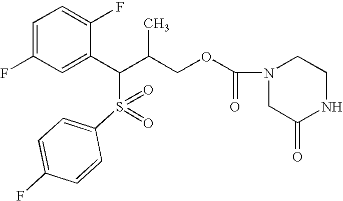 | Diastereomer 1, racemic | 1 | 4.11 | 471 |
| 131 | Analogous to Example 11 | 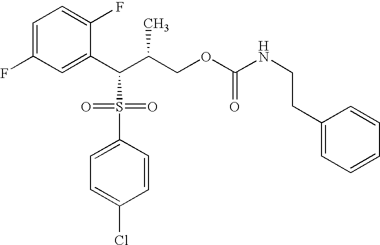 | Diastereomer 1, Enantiomer A | 9 | 3.86 | 508 |
| 132 | Analogous to Example 11 | 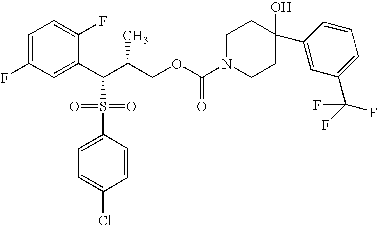 | Diastereomer 1, Enantiomer A | 3 | 4.43 | 632 |
| 133 | Analogous to Example 11 | 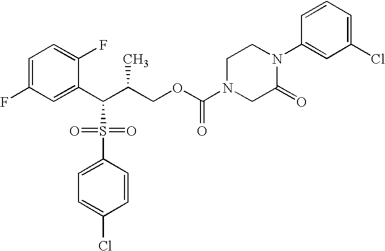 | Diastereomer 1, racemic | 3 | 4.15 | 597 |
| 134 | Analogous to Example 11 | 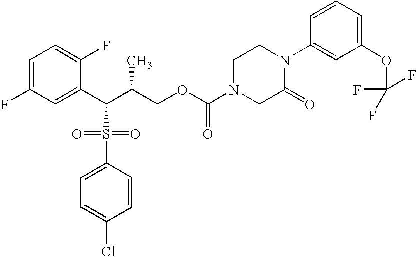 | Diastereomer 1, Enantiomer A | 6 | 4.11 | 647 |
| 135 | Analogous to Example 11 | 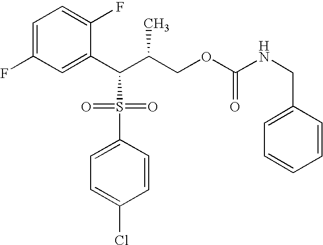 | Diastereomer mixture, racemic | 8 | 5.00 | 494 |
| 136 | Analogous to Example 11 | 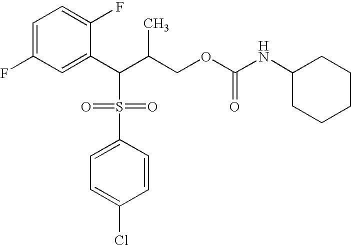 | Diastereomer mixture, racemic | 8 | 5.15 | 508 [M +Na]⁺ |
| 137 | Analogous to Example 11 | 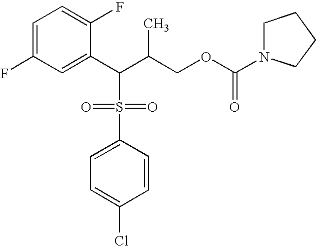 | Diastereomer mixture, racemic | 3 | 4.55 | 490 |
| 138 | Analogous to Example 11 | 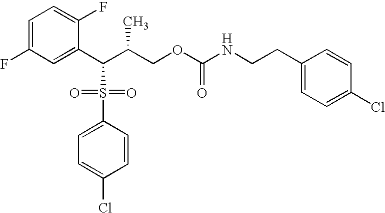 | Diastereomer 1, Enantiomer A | 9 | 4.05 | 542 |
| 139 | Analogous to Example 11 | 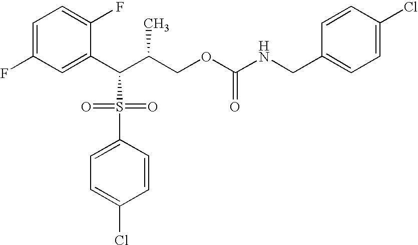 | Diastereomer 1, Enantiomer A | 8 | 5.21 | 528 |
| 140 | Analogous to Example 11 | 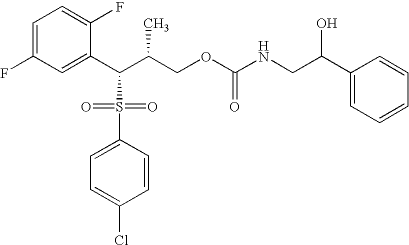 | Diastereomer 1, racemic | 3 | 3.90 | 524 |
| 141 | Analogous to Example 11 | 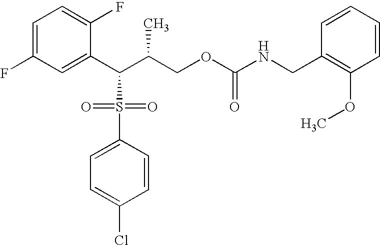 | Diastereomer 1, Enantiomer A | 9 | 3.81 | 524 |
| 142 | Analogous to Example 11 | 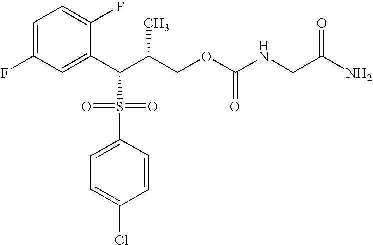 | Diastereomer 1, racemic | 8 | 3.98 | 461 |
| 143 | Analogous to Example 14 | 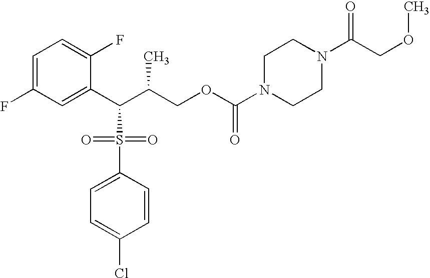 | Diastereomer 1, racemic | 8 | 4.31 | 545 |
| 144 | Analogous to Example 14 | 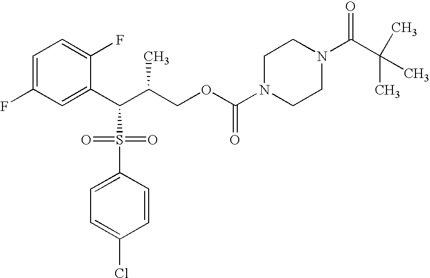 | Diastereomer 1, Enantiomer A | 3 | 4.09 | 557 |
| 145 | Analogous to Example 14 | 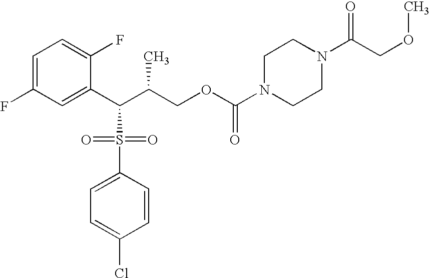 | Diastereomer 1, Enantiomer A | 6 | 3.70 | 545 |
| 146 | Analogous to Example 14 | 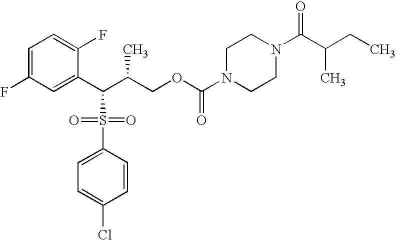 | Diastereomer 1, Enantiomer A | 6 | 3.94 | 557 |
| 147 | Analogous to Example 14 | 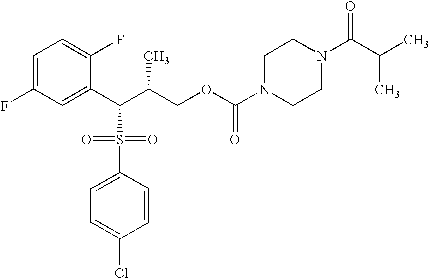 | Diastereomer 1, racemic | 3 | 3.88 | 543 |
| 148 | Analogous to Example 14 | 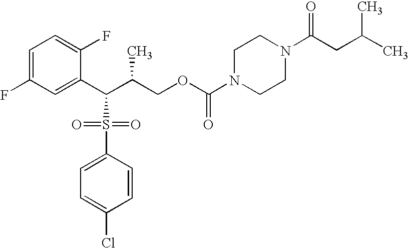 | Diastereomer 1, Enantiomer A | 6 | 3.98 | 557 |
| 149 | Analogous to Example 14 | 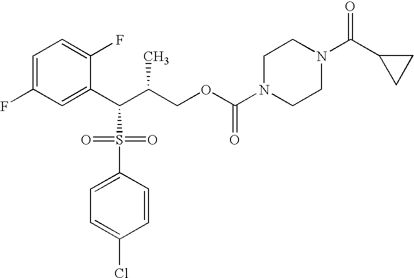 | Diastereomer 1, Enantiomer A | 3 | 3.80 | 541 |
| 150 | Analogous to Example 14 | 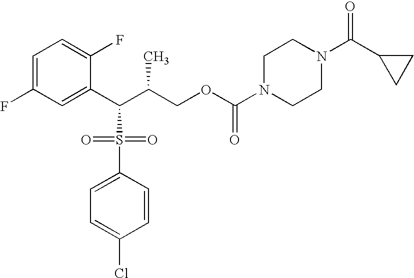 | Diastereomer 1, racemic | 3 | 3.82 | 541 |
| 151 | Analogous to Example 14 | 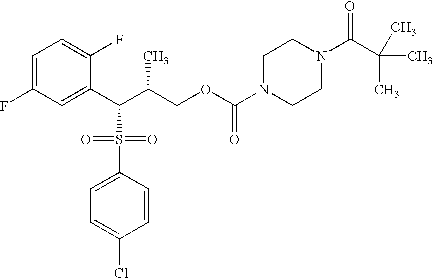 | Diastereomer 1, racemic | 8 | 4.85 | 557 |
| 152 | Analogous to Example 14 | 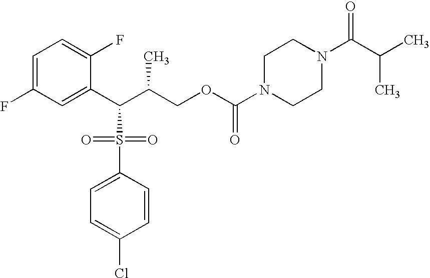 | Diastereomer 1, Enantiomer A | 6 | 3.90 | 543 |
| 153 | Analogous to Example 14 | 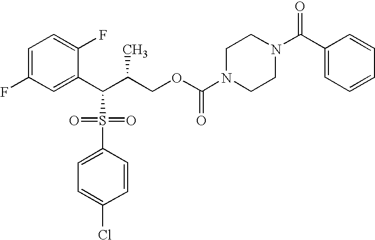 | Diastereomer 1, racemic | 3 | 4.00 | 577 |
| 154 | Analogous to Example 21 | 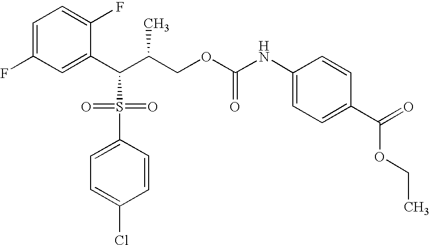 | Diastereomer 1, Enantiomer A | 5 | 4.54 | 552 |
| 155 | Analogous to Example 21 | 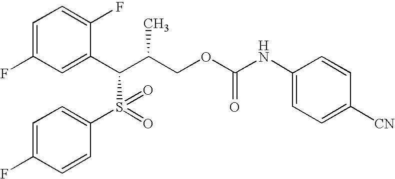 | Diastereomer 1, racemic | 1 | 4.92 | 489 |
| 156 | Analogous to Example 21 | 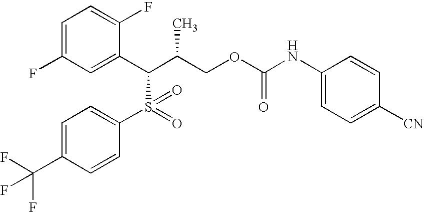 | Diastereomer 1, Enantiomer A | 1 | 5.09 | 539 |
| 157 | Analogous to Example 21 | 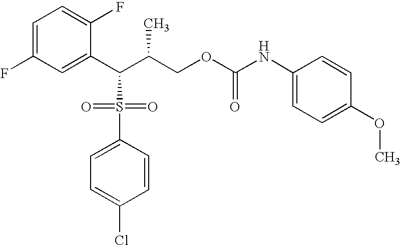 | Diastereomer 1, Enantiomer A | 9 | 3.77 | 510 |
| 158 | Analogous to Example 21 | 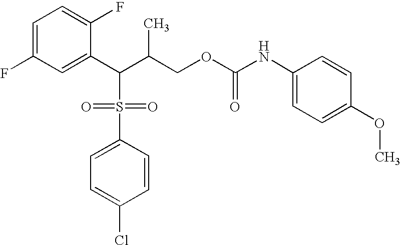 | Diastereomer mixture, racemic | 8 | 5.00 | 510 |
| 159 | Analogous to Example 21 | 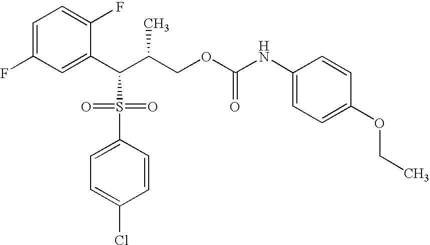 | Diastereomer 1, Enantiomer A | 9 | 3.92 | 524 |
| 160 | Analogous to Example 21 | 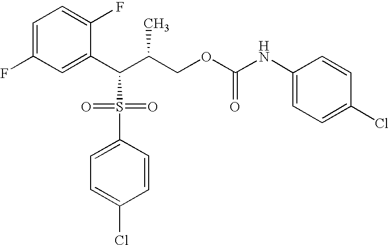 | Diastereomer 1, Enantiomer A | 9 | 4.07 | 514 |
| 161 | Analogous to Example 21 | 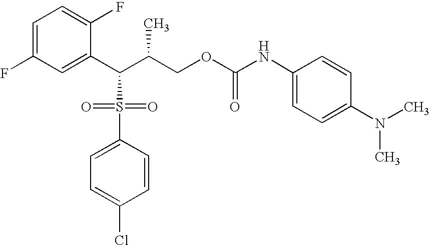 | Diastereomer 1, Enantiomer A | 7 | 4.24 | 523 |
| 162 | Analogous to Example 21 | 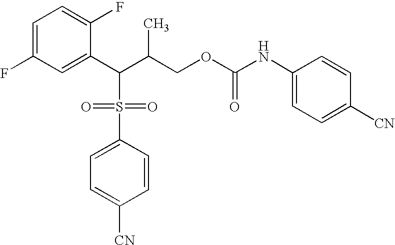 | Diastereomer mixture, racemic | 6 | 3.93 and 3.96 | 496 |
| 163 | Analogous to Example 21 | 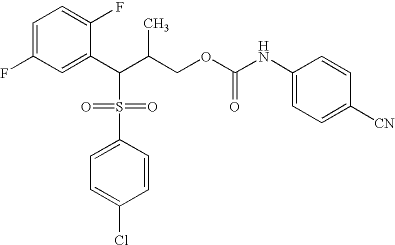 | Diastereomer mixture, racemic | 8 | 5.29 | 503 [M − H]⁺ |
| 164 | Analogous to Example 21 | 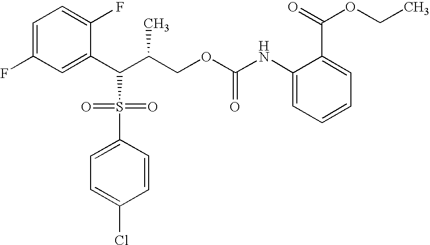 | Diastereomer 1, Enantiomer A | 5 | 4.75 | 552 |
| 165 | Analogous to Example 21 | 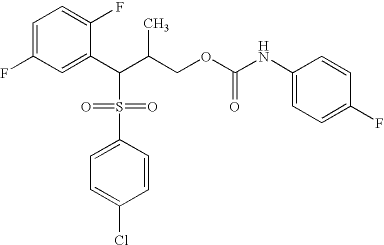 | Diastereomer mixture, racemic | 8 | 5.06 | 498 |
| 166 | Analogous to Example 19 | 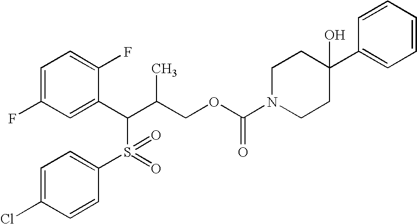 | racemic | 1 | 4.91 | 564 |
| 167 | Analogous to Example 19 | 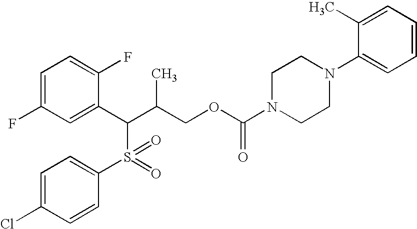 | racemic | 1 | 5.32 | DCI (NH₃); 563 |
| 168 | Analogous to Example 19 | 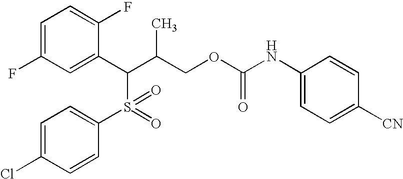 | Enantiomer A | 1 | 4.99 | DCI (NH₃); 522 [M +NH₄]⁺ |
| 169 | Analogous to Example 19 | 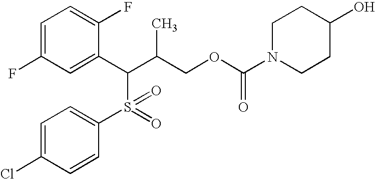 | racemic | 1 | 4.4 | 488 |
| 170 | Analogous to Example 19 | 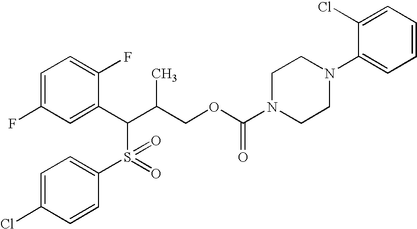 | racemic | 1 | 5.5 | 583 |

¹H-NMR data for:

Example 121 (200 MHz, DMSO-d₆): δ=8.05 (br. s, 1H), 7.90 (d, 2H), 7.80 (d, 2H), 7.45-7.30 (m, 1H), 7.25-7.10 (m, 1H), 7.10-6.90 (m, 1H), 4.90 (d, 1H), 3.95 (dd, 1H), 3.85-3.65 (m, 3H), 3.55-3.40 (m, 2H), 3.20-2.95 (m, 3H), 1.45 (d, 3H).

Example 130 (300 MHz, DMSO-d₆): δ=8.00 (br. s, 1H), 7.65 (dd, 2H), 7.40-7.25 (m, 3H), 7.20-7.10 (m, 1H), 7.05-6.95 (m, 1H), 4.75 (d, 1H), 3.95 (dd, 1H), 3.85-3.65 (m, 3H), 3.40 (br. s, 2H), 3.15 (br. s, 2H), 3.05-2.95 (m, 1H), 1.45 (d, 3H).

Example 166 (200 MHz, DMSO-d₆): δ=7.65 (d, 2H), 7.50-7.40 (m, 4H), 7.40-7.10 (m, 6H), 5.05 (s, 1H), 4.05 (br. t, 2H), 3.90-3.70 (m, 1H), 3.45-3.20 (m, 1H), 3.20-2.90 (m, 3H), 2.30-2.10 (m, 1H), 1.80 (s, 3H), 1.80-1.60 (m, 2H), 1.60-1.40 (m, 2H).

The in vitro effect of the compounds of the invention can be shown in the following assays:

Determination of the Inhibition of A-Beta Release in Cell Culture

a) Cell Culture

In order to be able to measure the inhibition of Aβ release, human cell lines (H4, HEK293) which stably overexpress the 695 amino acid-long, neuronal splice variant of human APP were generated. In order to increase the generated Aβ amount further, in addition a “Swedish” familiar Alzheimer's double mutation in which the lysine and methionine residues respectively at positions 595 and 596 of the molecule APP695 are replaced by the amino acids asparagine and leucine was introduced. The cells were cultivated in Dulbecco's modified Eagles medium (DMEM, with 4500 mg/l glucose; 110 mg/l sodium pyruvate); 5% by volume fetal calf serum (FCS); 1% nonessential amino acids) to which the geniticin G418 selection marker had been added [all cell culture methods were carried out by standard methods; Sambrook, J., Fritsch, E. F., and Maniatis, T. (1989), Molecular cloning: A laboratory manual. Cold Spring Harbour Laboratory Press]. In order to test the effect of substances on the inhibition of APP processing, about 20 000 cells were diluted in a 96 multititer plate. The next day, the culture medium was removed and replaced by biotin- and serum-free medium, in which the substances were diluted to reach a concentration of 10 μM with a dimethyl sulfoxide (DMSO) content of 0.5%. 0.5% DMSO served as control. For substances showing inhibition of the Aβ generation, additionally dose-effect relations were investigated by using different concentrations. After 16 h, the supernatant was removed and analyzed.

b) Detection of Aβ with the IGEN Analyzer

The total amount of Aβ was detected using the following components: 50 μl of cell culture supernatant were mixed with 25 μl of biotinylated antibody 4G8 (recognizes amino acid 17-25 of Aβ), 25 μl of ruthenium complex-labeled antibody 6E10 (recognizes the N terminus of Aβ) and 50 μl of magnetic streptavidin-coupled beads. Aβ40 was detected by using the following components: 50 μl of cell culture supernatant were mixed with 25 μl of biotinylated antibody G2-10 (recognizes the C terminus of Aβ40), 25 μl of ruthenium complex-labeled antibody W02 (recognizes the N terminus of Aβ), and 50 μl of magnetic streptavidin-coupled beads. In parallel, serial dilutions were made with synthetic A040. The samples were shaken at room temperature and then measured using an IGEN analyzer. Typically, each sample was measured three times in at least two independent experiments. The antibodies and solutions used were prepared according to the instructions of the manufacturer of the analyzer, IGEN, Inc. (Gaitersburg, Md., USA). The measurement was likewise carried out as stated by the manufacturer.

Exemplary embodiments 10-4, 11-14, 42, 43, 45-56, 95, 100, 102-104 and 143-146 show IC₅₀ values between 10 and 100 nM in this test.

### EXEMPLARY EMBODIMENTS OF PHARMACEUTICAL COMPOSITIONS

The compounds of the invention can be converted into pharmaceutical preparations in the following ways:

Tablet:

Composition:

100 mg of the compound of Example 1, 50 mg of lactose (monohydrate), 50 mg of corn starch (native), 10 mg of polyvinylpyrrolidone (PVP 25) and 2 mg of magnesium stearate.

Tablet weight 212 mg. Diameter 8 mm, radius of curvature 12 mm.

Production:

A mixture of active ingredient, lactose and starch is granulated with a 5% strength solution (m/m) of the PVP in water. The granules are dried and then mixed with the magnesium stearate for 5 min. This mixture is compressed in a conventional tablet press (see above for format of the tablet). A compressive force of 15 kN is used as guideline for the compression.

Suspension which can be Administered Orally:

Composition:

1000 mg of the compound of Example 1, 1000 mg of ethanol (96%), 400 mg of Rhodigel (xanthan gum from FMC, Pennsylvania, USA) and 99 g of water.

10 ml of oral suspension correspond to a single dose of 100 mg of the compound of the invention.

Production:

The Rhodigel is suspended in ethanol, and the active ingredient is added to the suspension. The water is added while stirring. The mixture is stirred for about 6 h until the swelling of the Rhodigel is complete.

1. A compound of the formula 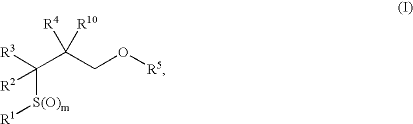
in which
R¹ and R² are independently of one another phenyl which is optionally substituted by radicals selected from the group of halogen, cyano, trifluoromethyl, trifluoromethoxy, C₁-C₆-alkyl, C₃-C₈-cycloalkyl, C₁-C₆-alkoxy and C₁-C₆-alkylthio,
R³ and R⁴ are independently of one another hydrogen, C₁-C₆-alkyl or C₃-C₈-cycloalkyl, which are optionally substituted by hydroxy,
m is 1 or 2,
R⁵ is hydrogen,
or a radical of the formula CO—NR⁶R⁷ in which
R⁶ and R⁷ are independently of one another hydrogen, C₁-C₆-alkyl, C₃-C₈-cycloalkyl, benzyl, phenethyl, phenyl or 5- to 6-membered heteroaryl, where C₁-C₆-alkyl, C₃-C₈-cycloalkyl, phenyl or 5- to 6-membered heteroaryl are optionally substituted by radicals independently of one another selected from the group of hydroxy, halogen, C₁-C₆-alkylamino, aminosulfonyl, aminocarbonyl, cyano, formamido, acetamido, C₁-C₆-alkyl, C₁-C₆-alkoxy, C₃-C₈-cycloalkyl, hydroxycarbonyl, C₁-C₆-alkoxycarbonyl and 5- to 6-membered heteroaryl, and
benzyl and phenethyl are optionally substituted by radicals independently of one another selected from the group of hydroxy, halogen, aminocarbonyl, C₁-C₆-alkylamino, aminosulfonyl, cyano, formamido, acetamido, C₁-C₆-alkyl, C₁-C₆-alkoxy, C₃-C₈-cycloalkyl and 5- to 6-membered heteroaryl,
or in which
the group NR⁶R⁷
is a 4- to 10-membered heterocyclyl radical which is linked via the nitrogen atom and which is optionally substituted by radicals independently of one another selected from the group of C₁-C₆-alkyl, C₁-C₆-alkoxy, 1,3-dioxapropane-1,3-diyl, 1,4-dioxabutane-1,4-diyl, oxo, C₃-C₈-cycloalkyl, hydroxy, halogen, cyano, C₁-C₆-alkylcarbonyl, C₃-C₈-cycloalkylcarbonyl, phenylcarbonyl, formamido, aminosulfonyl, C₁-C₆-alkoxycarbonyl, aminocarbonyl, phenyl and 5- to 6-membered heteroaryl,
where phenyl is optionally substituted by radicals independently of one another selected from the group of halogen, cyano, trifluoromethyl, trifluoromethoxy, C₁-C₆-alkyl, C₁-C₆-alkoxy and C₁-C₆-alkylsulfonamino, and
C₁-C₆-alkyl is optionally substituted by radicals independently of one another selected from the group of hydroxy, C₁-C₆-alkoxy, phenyl and 5- to 6-membered heteroaryl, and
C₁-C₆-alkylcarbonyl is optionally substituted by radicals independently of one another selected from the group of hydroxy and C₁-C₆-alkoxy,
and where 4- to 10-membered heterocyclyl is optionally benzo-substituted, or
a radical of the formula CO—OR⁸ in which
R⁸ is C₁-C₆-alkyl or C₃-C₈-cycloalkyl, which are optionally substituted by radicals independently of one another selected from the group of hydroxy, halogen, aminosulfonyl, aminocarbonyl, cyano, formamido, acetamido, C₁-C₆-alkyl, C₁-C₆-alkoxy, C₃-C₈-cycloalkyl, C₁-C₆-alkylcarbonyl, phenyl and 5- to 6-membered heteroaryl, or
a radical of the formula CO—R⁹ in which
R⁹ is C₁-C₆-alkyl, C₃-C₈-cycloalkyl, C₆-C₁₀-aryl or 5- to 10-membered heteroaryl, which are optionally substituted by radicals selected from the group of hydroxy, hydroxycarbonyl, halogen, aminosulfonyl, carboxamido, cyano, formamido, acetamido, C₁-C₆-alkyl, C₁-C₆-alkoxy, C₃-C₈-cycloalkyl, C₁-C₆-alkylcarbonyl, phenyl and 5- to 6-membered heteroaryl,
R¹⁰ is hydrogen or C₁-C₆-alkyl,
and the salts, solvates and solvates of the salts thereof.

2. A compound of the formula (I) in which
R¹ and R² are independently of one another phenyl which is optionally substituted by radicals selected from the group of halogen, cyano, trifluoromethyl,
R³ and R⁴ are independently of one another hydrogen, C₁-C₄-alkyl or C₃-C₆-cycloalkyl, which are optionally substituted by hydroxy,
m is 1 or 2,
R⁵ is hydrogen, or
a radical of the formula CO—NR⁶R⁷ in which
R⁶ is hydrogen, C₁-C₄-alkyl,
R⁷ is hydrogen, C₁-C₄-alkyl, C₃-C₆-cycloalkyl, benzyl, phenethyl or phenyl, where C₁-C₄-alkyl, C₃-C₆-cycloalkyl and phenyl are optionally substituted by radicals independently of one another selected from the group of hydroxy, halogen, aminocarbonyl, hydroxycarbonyl, cyano, C₁-C₄-alkylamino, C₁-C₄-alkyl, C₁-C₄-alkoxy, C₃-C₆-cycloalkyl, C₁-C₄-alkoxycarbonyl and 5- to 6-membered heteroaryl, and
benzyl and phenethyl are optionally substituted by radicals independently of one another selected from the group of hydroxy, halogen, aminocarbonyl, cyano, C₁-C₄-alkylamino, C₁-C₄-alkyl, C₁-C₄-alkoxy, C₃-C₆-cycloalkyl and 5- to 6-membered heteroaryl,
or in which
the group NR⁶R⁷
is a 5- to 6-membered heterocyclyl radical which is linked via the nitrogen atom and which is optionally substituted by radicals independently of one another selected from the group of C₁-C₄-alkyl, C₁-C₄-alkoxy, 1,3-dioxapropane-1,3-diyl, 1,4-dioxabutane-1,4-diyl, oxo, C₃-C₆-cycloalkyl, hydroxy, halogen, C₁-C₄-alkylcarbonyl, C₃-C₆-cycloalkylcarbonyl, phenylcarbonyl, C₁-C₄-alkoxycarbonyl, phenyl and 5- to 6-membered heteroaryl,
where phenyl is optionally substituted by radicals independently of one another selected from the group of halogen, cyano, trifluoromethyl, trifluoromethoxy, C₁-C₄-alkyl, C₁-C₄-alkoxy and C₁-C₄-alkylsulfonamino, and
C₁-C₄-alkyl is optionally substituted by radicals independently of one another selected from the group of hydroxy and phenyl, and
C₁-C₄-alkylcarbonyl is optionally substituted by radicals independently of one another selected from the group of hydroxy and C₁-C₄-alkoxy, or
a radical of the formula CO—R⁹ in which
R⁹ is C₁-C₄-alkyl, C₃-C₈-cycloalkyl, phenyl or 5- to 6-membered heteroaryl, which are optionally substituted by radicals selected from the group of hydroxy, hydroxycarbonyl, halogen, cyano, acetamido, C₁-C₄-alkyl, C₁-C₄-alkoxy, C₃-C₆-cycloalkyl, C₁-C₄-alkylcarbonyl, phenyl and 5- to 6-membered heteroaryl,
R¹⁰ is hydrogen or C₁-C₄-alkyl,
and the salts, solvates and solvates of the salts thereof.

3. A compound of the formula (I) in which
R¹ is phenyl which is optionally substituted by radicals selected from the group of fluorine, chlorine, bromine, cyano, trifluoromethyl,
R² is phenyl which is optionally substituted by fluorine,
R³ is hydrogen or C₁-C₄-alkyl,
R⁴ is hydrogen or C₁-C₄-alkyl which is optionally substituted by hydroxy
R⁵ is hydrogen, or
a radical of the formula CO—NR⁶R⁷ in which
R⁶ is hydrogen, C₁-C₄-alkyl,
R⁷ is C₁-C₄-alkyl, C₃-C₆-cycloalkyl, benzyl, phenethyl or phenyl, where C₁-C₄-alkyl, C₃-C₆-cycloalkyl, and phenyl are optionally substituted by radicals independently of one another selected from the group of hydroxy, fluorine, chlorine, aminocarbonyl, hydroxycarbonyl, cyano, dimethylamino, methoxy, ethoxy, C₁-C₄-alkoxycarbonyl or thienyl, and
benzyl and phenethyl are optionally substituted by radicals independently of one another selected from the group of hydroxy, fluorine, chlorine, aminocarbonyl, cyano, dimethylamino, methoxy, ethoxy or thienyl,
or in which
the group NR⁶R⁷
is a 5- to 6-membered heterocyclyl radical which is linked via the nitrogen atom and which is optionally substituted by radicals independently of one another selected from the group of C₁-C₄-alkyl, 1,3-dioxapropane-1,3-diyl, 1,4-dioxabutane-1,4-diyl, oxo, hydroxy, C₁-C₄-alkylcarbonyl, C₃-C₆-cycloalkylcarbonyl, phenylcarbonyl, C₁-C₄-alkoxycarbonyl, phenyl and 6-membered heteroaryl,
where phenyl is optionally substituted by radicals independently of one another selected from the group of fluorine, chlorine, cyano, trifluoromethyl, trifluoromethoxy, C₁-C₄-alkyl, C₁-C₄-alkoxy and C₁-C₄-alkylsulfonamino, and
C₁-C₄-alkyl is optionally substituted by radicals independently of one another selected from the group of hydroxy and phenyl, and
C₁-C₄-alkylcarbonyl is optionally substituted by radicals independently of one another selected from the group of hydroxy and methoxy, or
a radical of the formula CO—R⁹ in which
R⁹ is phenyl,
R¹⁰ is hydrogen or C₁-C₃-alkyl,
and the salts, solvates and solvates of the salts thereof.

4. A compound as claimed in claim 1, of the following formula 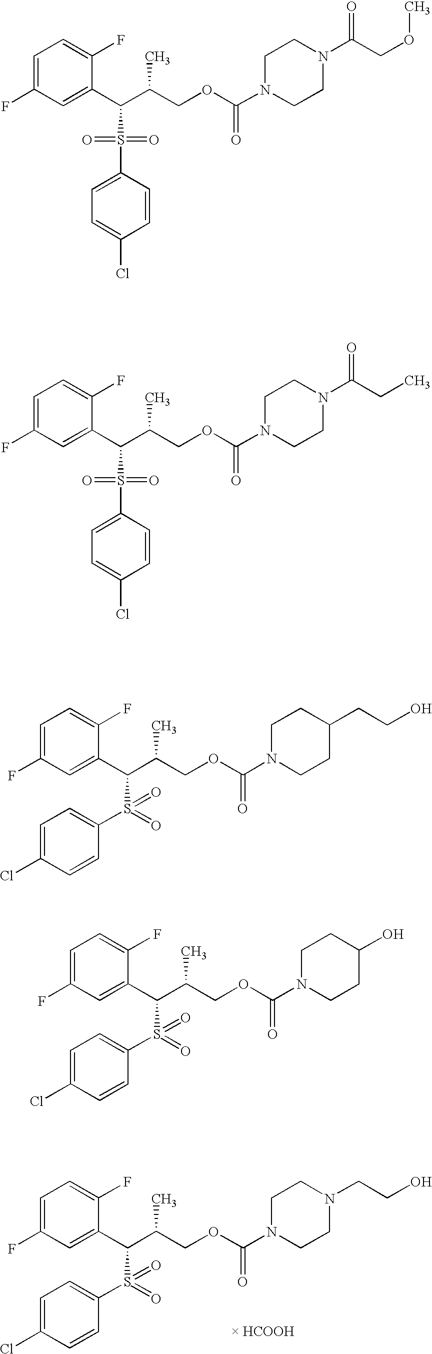 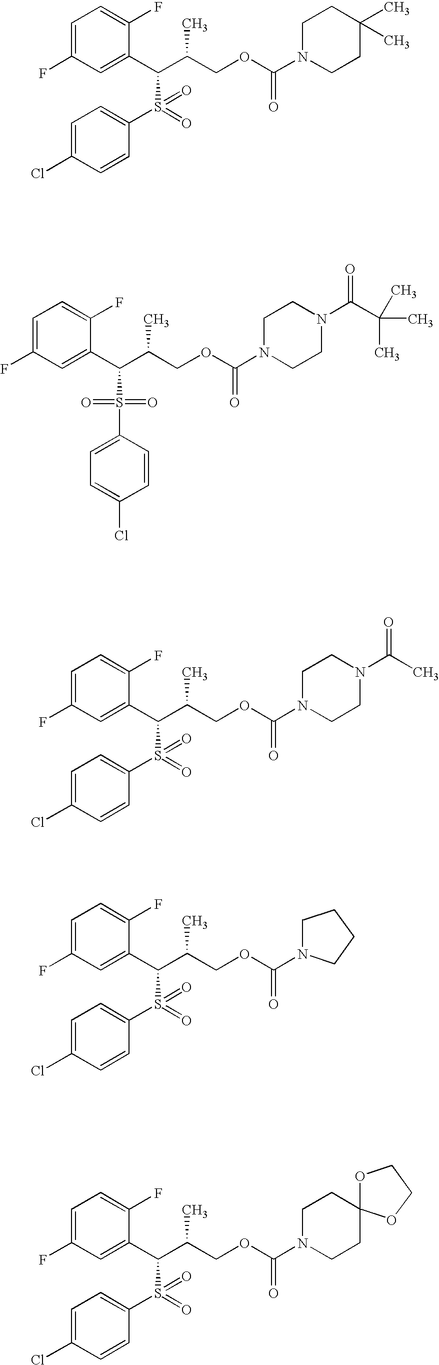 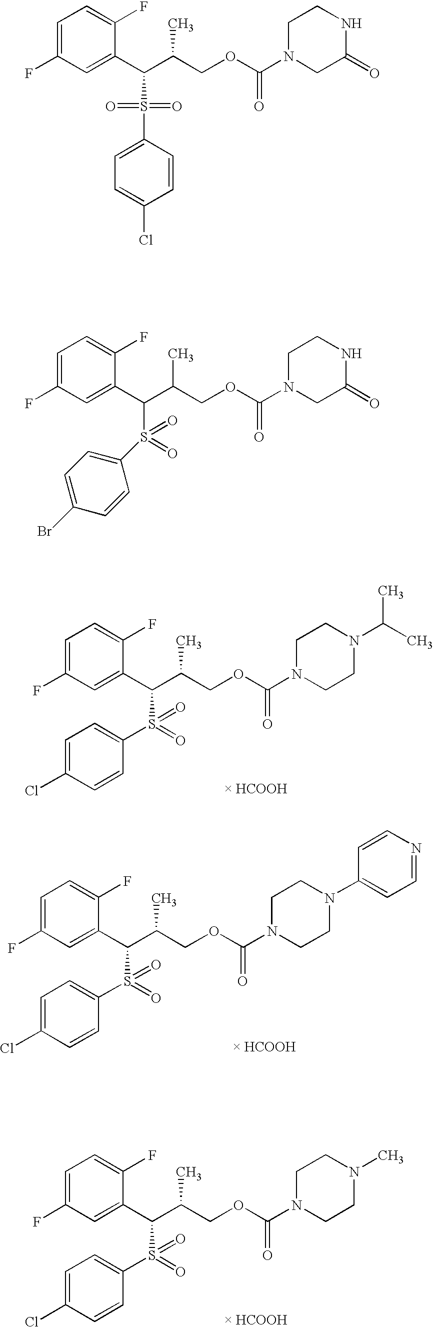 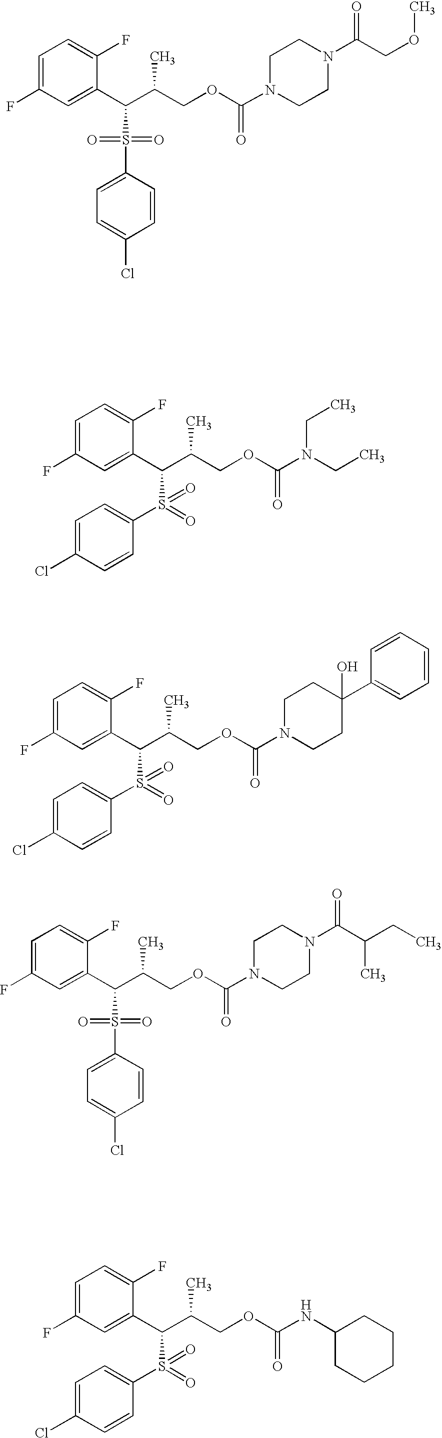 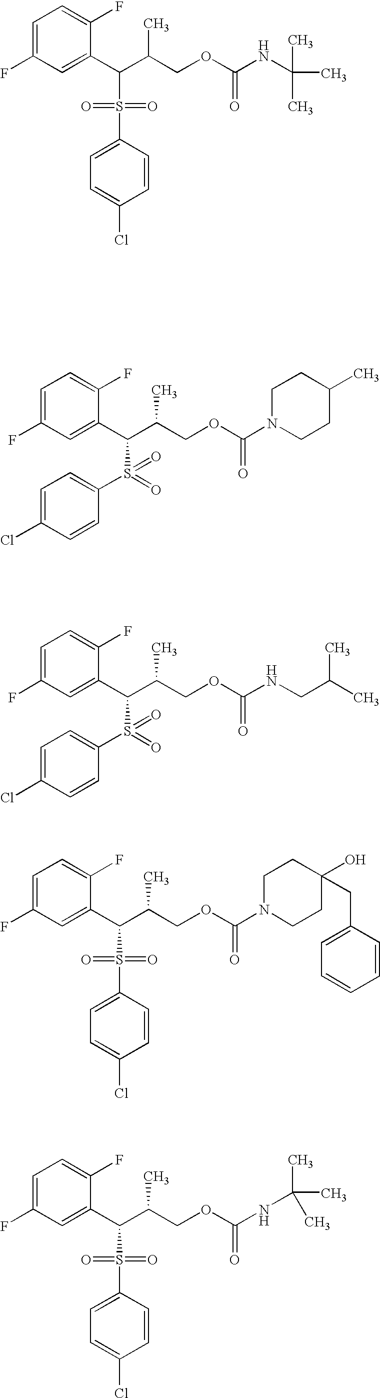 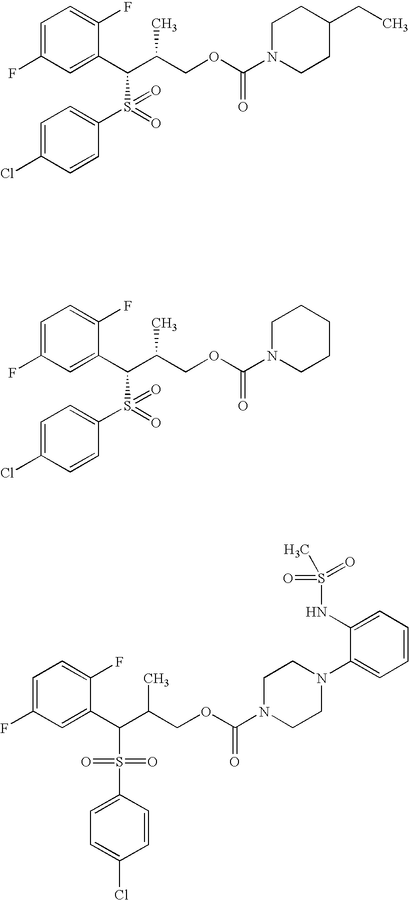
and the salts, solvates and solvates of the salts thereof.

5. A compound as claimed in claim 1, of the following formula 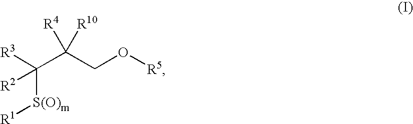
in which
R¹ and R² are independently of one another phenyl, which is optionally substituted by radicals selected from the group of halogen, cyano, trifluoromethyl, trifluoromethoxy, C₁-C₆-alkyl, C₃-C₈-cycloalkyl, C₁-C₆-alkoxy and C₁-C₆-alkylthio,
R³ and R⁴ are independently of one another hydrogen, C₁-C₆-alkyl or C₃-C₈-cycloalkyl,
m is 1 or 2, and
R⁵ is hydrogen,
is a radical of the formula CO—NR⁶R⁷
in which R⁶ and R⁷ are independently of one another hydrogen, C₁-C₆-alkyl, C₃-C₈-cycloalkyl, phenyl or 5- to 6-membered heteroaryl, or
in which the group NR⁶R⁷ is a 4- to 10-membered heterocyclyl radical which is linked via a nitrogen atom,
where alkyl, cycloalkyl, phenyl, heteroaryl and heterocyclyl are optionally substituted by radicals selected from the group of hydroxy, halogen, aminosulfonyl, carboxamido, cyano, formamido, acetamido, C₁-C₆-alkyl, C₁-C₆-alkoxy, C₃-C₈-cycloalkyl, C₁-C₆-alkanoyl, phenyl and 5- to 6-membered heteroaryl,
and where heterocyclyl is optionally is benzo-substituted,
is a radical of the formula CO—OR⁸
in which R⁸ is C₁-C₆-alkyl or C₃-C₈-cycloalkyl,
where alkyl and cycloalkyl are optionally substituted by radicals selected from the group of hydroxy, halogen, aminosulfonyl, carboxamido, cyano, formamido, acetamido, C₁-C₆-alkyl, C₁-C₆-alkoxy, C₃-C₈-cycloalkyl, C₁-C₆-alkanoyl, phenyl and 5- to 6-membered heteroaryl, or
is a radical of the formula CO—R⁹,
in which R⁹ is C₁-C₆-alkyl, C₃-C₈-cycloalkyl, C₆-C₁₀-aryl or 5- to 10-membered heteroaryl,
where alkyl, cycloalkyl, aryl and heteroaryl are optionally substituted by radicals selected from the group of hydroxy, halogen, aminosulfonyl, carboxamido, cyano, formamido, acetamido, C₁-C₆-alkyl, C₁-C₆-alkoxy, C₃-C₈-cycloalkyl, C₁-C₆-alkanoyl, phenyl and 5- to 6-membered heteroaryl,
and the salts, solvates and solvates of the salts thereof.

6. A compound as claimed in claim 1, where
R¹ is 2-fluorophenyl which is optionally additionally substituted once to twice by radicals selected from the group of fluorine, chlorine, cyano, trifluoromethyl, methyl and ethyl,
R² is 4-chlorophenyl which is optionally additionally substituted once to twice by radicals selected from the group of fluorine, chlorine, cyano, trifluoromethyl, methyl and ethyl,
R³ is hydrogen,
R⁴ is hydrogen or C₁-C₄-alkyl,
m is 1 or 2, and
R⁵ is a radical of the formula CO—NR⁶R⁷,
in which R⁶ and R⁷ are independently of one another hydrogen, C₁-C₆-alkyl, C₃-C₈-cycloalkyl or benzyl, or
in which the group NR⁶R⁷
is pyrrolidin-1-yl, piperidin-1-yl, morpholin-1-yl, thiomorpholin-1-yl, piperazin-1-yl, 4-methylpiperazin-1-yl or 4-ethylpiperazin-1-yl,
and the salts, solvates and solvates of the salts thereof.

7. A process for preparing compounds as claimed in claim 1, of the formula (I), characterized in that
[A] compounds of the formula 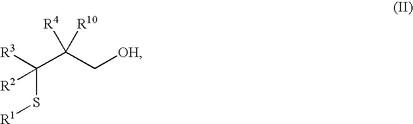
in which R¹ to R⁴ and R¹⁰ have the meanings indicated in claim 1, are first converted with appropriate equivalents of a suitable oxidizing agent such as, for example, peroxides or peracids, preferably meta-chloroperbenzoic acid (mCPBA) into compounds of the formula 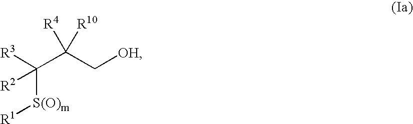
in which R¹ to R⁴, R¹⁰ and m have the meanings indicated in claim 1,
and the latter are then reacted in an acylation step, where appropriate in the presence of a base, with a compound of the formula
R^{5a}—X (III),
in which
R^{5a} has the meanings indicated above for R⁵ with the exception of hydrogen, and
X is a suitable leaving group such as, for example, halogen, or
[B] compounds of the formula (II) are first converted with a compound of the formula (III), where appropriate in the presence of a base, into compounds of the formula 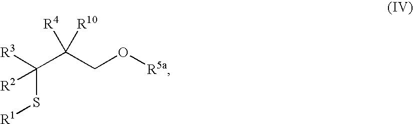
in which
R¹ to R⁴, R^{5a} and R¹⁰ have the meanings indicated above and in claim 1,
and the latter are then reacted with appropriate equivalents of a suitable oxidizing agent, preferably meta-chloroperbenzoic acid, or
[C] compounds of the formula 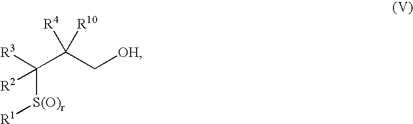
in which
R¹ to R⁴ and R¹⁰ have the meanings indicated in claim 1, and
r is zero, 1 or 2,
are first reacted, where appropriate in the presence of a base, with a compound of the formula 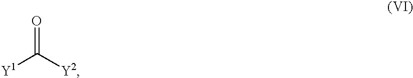
in which
Y¹ and Y² are identical or different and are a suitable leaving group such as, for example, halogen, —OCCl₃ or a group of the formula 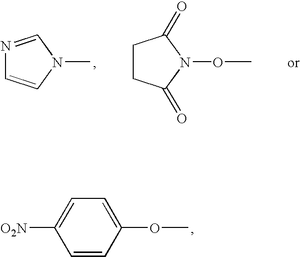
to give compounds of the formula 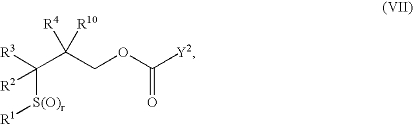
in which
R¹ to R⁴, R¹⁰, r and Y² have the meanings indicated above and in claim 1,
the latter are then, where appropriate in the presence of a base and/or of a suitable catalyst, converted with a compound of the formulae 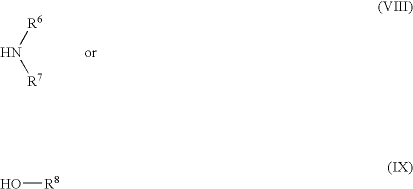
in which
R⁶, R⁷ and R⁸ have the meanings indicated above,
into compounds of the formulae 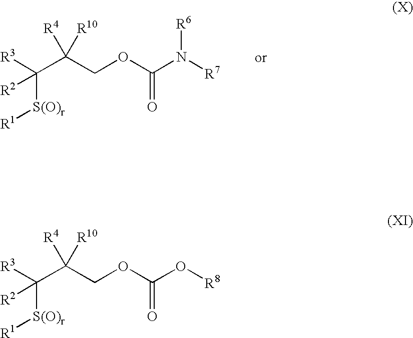
in which
R¹ to R⁴, R⁶ to R⁸, R¹⁰ and r have the meanings indicated above and in claim 1,
and the latter are then, where r is zero, reacted with appropriate equivalents of a suitable oxidizing agent, preferably meta-chloroperbenzoic acid,
and the resulting compounds (I) and (Ia) are converted where appropriate with the appropriate solvents and/or bases or acids into their solvates, salts and/or solvates of the salts.

8. A compound as claimed in claim 1 for the treatment and/or prophylaxis of diseases.

9. A medicament comprising at least one compound as claimed in claim 1 in combination with at least one pharmaceutically acceptable, pharmaceutically acceptable carrier or excipient.

10. The use of compounds as claimed in claim 1 for producing a medicament for the treatment and/or prophylaxis of Alzheimer's disease.

11. The medicament as claimed in claim 9 for the treatment and/or prophylaxis of Alzheimer's disease.

12. A method for controlling Alzheimer's disease in humans and animals by administering an effective amount of at least one compound as claimed in claim 1.
